(19) United States
(12) Patent Application Publication
(10) Pub. No.: US 2002/0012710 A1
(43) Pub. Date: Jan. 31, 2002

(54) Pomegranate products useful in improving health and methods of use thereof

(75) Inventor: Ephraim Philip Lansky, Haifa (IL)

Correspondence Address: DR. MARK FRIEDMAN LTD., C/O BILL POLKINGHORN -DISCOVERY DISPATCH, 9003 FLORIN WAY, UPPER MALBORO, MD, 20772, US

(73) Assignee: RIMONEST LTD.

(21) Appl. No.: 09/859,431

(22) Filed: May 18, 2001

Related U.S. Application Data
(63) Continuation-in-part of application No. PCT/IL00/00800, filed on Oct. 6, 2000.
(60) Provisional application No. 60/167,694, filed on Nov. 29, 1999.

(51) Int. Cl. A61K 35/78; C07J 1/00

(52) U.S. Cl. 424/725; 424/776; 424/777; 552/625; 549/408; 552/653

(57) ABSTRACT

A mixture of a pomegranate seed oil product and a pomegranate juice product and a pharmaceutical composition containing same. The mixture, which is a cancer chemopreventive, includes a pomegranate seed oil product and pomegranate juice product. The pharmaceutical composition includes physiologically active amounts of pomegranate seed oil product, a pomegranate juice product and a pharmaceutically acceptable carrier. According to some disclosed embodiments, a pomegranate peel product is further included. Further disclosed is a selective estrogen receptor modulator and other biologically active compounds derived from pomegranates as well as methods of use thereof.

This application is a CIP of PCT application IL00/00800 filed Oct. 6, 2000, which claimed priority from U.S. application 60/167,694 filed Nov. 29, 1999.

### FIELD AND BACKGROUND OF THE INVENTION

The present invention relates to pomegranate products useful in improving health and methods of use thereof. The present invention further relates to a mixture of a pomegranate seed oil product and a pomegranate juice product and to a pharmaceutical composition containing same. More particularly, mixtures of the present invention have cancer preventing properties and a pharmaceutical composition containing the mixture may be advantageously employed to treat or prevent a variety of conditions, including but not limited to, cancer, Alzheimer's disease, climacteria, benign prostatic hyperplasia and estrogen deficiency. More particularly, the present invention relates to selective estrogen receptor modulators (SERMS) derived from pomegranate products and to methods of selectively modulating estrogen receptors using pomegranate products or combinations thereof. The present invention further relates to novel methods of producing compositions containing physiologically active amount of 17 alpha estradiol, gamma tocopherol or campesterol from pomegranate as well as to methods of purifying these materials from pomegranate. The present invention further relates to the administration of pomegranate seed oil to a subject to exert an effect on a physiologic process of the subject. The present invention further relates to novel methods for retarding atherosclerosis and for retarding cellular aging using a pomegranate product.

Pomegranate (Punica granatum) has long been recognized as a fruit with many benefits for health.¹ The plant is botanically unique, having actually only one true botanical relative, the pomegranate precursor, Punica protopunica, restricted to the isolated island Socotra off the coast of Yemen. Corresponding to this botanical uniqueness is a parallel distinctiveness in terms of biochemistry. For example, pomegranate has long been recognized as the richest plant source of the female steroid hormone estrone,² and recently, the male hormone testosterone and another female steroid, estriol, have also been discovered in pomegranate seed oil.³ A wide range of polyphenolic compounds including flavonoids, anthocyanins and tannins have been characterized both in pomegranate juice⁴ and pericarp.5 Further, concentrations of these polyphenols extracted both from the fermented juice and the oil have been shown to be potently antioxidant in vitro and to additionally inhibit the eicosanoid enzyme lipoxygenase, and in the case of the polyphenols extracted from pomegranate seed oil, to also be significantly inhibitory of another eicosanoid pathway enzyme, cyclooxygenase.⁶ ¹ Frawley, D and Lad, V. The Yoga of Herbs: An Ayurvedic Guide to Herbal Medicine, Lotus Press, Twin Lakes, WI 1986. ² Moneam, N. M. A., El Sharaky, A. S., and Badreldin, M. M. Oestrogen content of pomegranate seeds. Journal of Chromotography 438: 438-442, 1988. ³ Abd El Wahab, S. M., El Fiki, S. F., Mostafa, S. F. and Hassan, A. E. B. Characterization of certain steroid hormones in Punica granatum L. seeds. Bulletin of the Faculty of Pharmacy of Cairo University 36(1): 11-15, 1998. ⁴ Artik, N., Cemeroglu, B., Burakami, H., and Mori, T. Determination of phenolic compounds in pomegranate juice by HPLC. Fruit Process 8 (12): 492-499, 1998. ⁵ Ben Nasr, C., Ayed, N., and Metche, M. Quantitative determination of the polyphenolic content of pomegranate peel. Z Lebensm Unters Forsch 203 (4): 374-378, 1996. ⁶ Schubert, S. Y., Lansky, E. P., and Neeman, I. Antioxidant and eicosanoid enzyme inhibition properties of pomegranate seed oil and fermented juice flavonoids. Journal of Ethnopharmacology 66 (1): 11-17, 1999.

However, pomegranate products have not previously been demonstrated to affect activity of aromatase, an enzyme which catalyzes the transformation of andostenedione to estrone, and of testosterone to estradiol. In addition, there has been no experimental analysis of the ability of pomegranate fractions to interfere with the estrogenic activity of a compound known to exert estrogenic activity, namely 17-beta estradiol. Further, there is no report of the ability of pomegranate products to selectively modulate estrogen receptors. Compounds with the ability to selectively modulate estrogen receptors are known collectively as “SERMs”. SERMs including Tamoxifen, Clomiphene and Raloxifene, exhibit demonstrable importance in a number of medical contexts including, but not limited to, post menopausal osteoporosis, hormone dependent cancers, cardiovascular disease and induction of ovulation in sub-fertile women. (Goldstein, S. R. et al. (2000) Hum Reprod Update 6(3): 212-224; Diez, J. L. (2000) Hum Reprod Update 6(3): 255-258; Brown, P. H. and S. M. Lippman (2000) Breast Cancer Res Treat 62(1): 1-17; and Burger, H. G. (2000) Horm Res 53 (Suppl. 3): 25-29).

Demonstration of these capabilities would suggest potential utility in the prevention and treatment of cancer, including, but not limited to, estrogen dependent cancers such as those of breast and prostate, as well as for colon cancer. Such utility could easily extend beyond cancer to include a wide range of physiologic benefits.

There is thus a widely recognized need for, and it would be highly advantageous to have, pomegranate products useful in improving health and methods of use thereof which could be safely used to improve the human condition.

### SUMMARY OF THE INVENTION

According to one aspect of the present invention there is provided a cancer chemopreventive mixture. The mixture comprises a pomegranate seed oil product and a pomegranate juice product.

According to another aspect of the present invention there is provided a pharmaceutical composition. The composition comprises physiologically active amounts of a pomegranate seed oil product and a pomegranate juice product and a pharmaceutically acceptable carrier.

According to further features in preferred embodiments of the invention described below, a pomegranate peel product is further included.

According to still further features in the described preferred embodiments the pomegranate seed oil product is the result of a process selected from the group consisting of expeller pressing, supercritical fluid extraction with carbon dioxide, and lyophilization.

According to still further features in the described preferred embodiments the pomegranate seed oil product is produced from a material selected from the group consisting of pomegranate seeds and pomegranate seed cake.

According to still further features in the described preferred embodiments the pomegranate seed oil product is selected from the group consisting of pomegranate seed oil and a non saponifiable fraction thereof.

According to still further features in the described preferred embodiments the pomegranate juice product comprises at least one item selected from the group consisting of pomegranate juice, fermented pomegranate juice, dried pomegranate juice, dried fermented pomegranate juice, partially fermented pomegranate juice, partially dried pomegranate juice, partially fermented partially dried pomegranate juice, reduced pomegranate juice, partially reduced pomegranate juice and lyophylysates thereof and supercritical fluid extracts thereof the supercritical fluids so employed selected from the group of CO₂, water, ethanol, methanol and all other chemical solvents.

According to still further features in the described preferred embodiments the mixture is provided in a form selected from the group consisting of a liquid, a powder, granules, a tablet, a capsule, a gel-cap, an ointment, a lotion, a bath gel, a cream, a chewing gum, a food, a candy, an emulsion and a suppository.

According to still further features in the described preferred embodiments the cancer is a hormone dependent cancer.

According to still further features in the described preferred embodiments the hormone dependent cancer is selected from the group consisting of breast cancer and prostate cancer.

According to still further features in the described preferred embodiments the pomegranate peel product is selected from the group consisting of pomegranate peel residue present in pomegranate juice as a result of a juicing process, an aqueous extract of pomegranate peel, an alcohol extract of pomegranate peel, an extract performed with an organic solvent which is not an alcohol, and a supercritical fluid extract of pomegranate peel. The supercritical fluid extract may be conducted with, for example CO₂, ethanol, methanol, water, or combinations thereof.

According to still further features in the described preferred embodiments the pomegranate peel product contains at least one item selected from the group consisting of quercetin, kaempferol, luteolin, derivatives thereof and combinations thereof.

the pharmaceutical composition is efficaciously employed for treatment of a medical condition.

According to still further features in the described preferred embodiments the medical condition is selected from the group consisting of cancer, Alzheimer's disease, climacteria, benign prostatic hyperplasia and estrogen deficiency.

According to still further features in the described preferred embodiments the treatment is selected from the group consisting of a prophylactic treatment, a palliative treatment and a therapeutic treatment.

According to still further features in the described preferred embodiments the physiologic activity results from inhibition of an enzyme selected from the group consisting of aromatase and 17-beta-hydroxysteroid dehdrogenase (HSD) type 1.

According to still further features in the described preferred embodiments the active ingredients of the mixture or pharmaceutical composition comprise dealcoholized concentrated pomegranate wine, aqueous extract of pomegranate pericarp, and seed cake extract, supercritical fluid polyphenol-rich extracts of the preceding and combinations thereof.

According to still further features in the described preferred embodiments the physiologically active ingredients comprise approximately 70% dealcoholized concentrated pomegranate wine, approximately 10% aqueous extract of pomegranate pericarp, and approximately 20% seed cake extract.

According to still further features in the described preferred embodiments the physiologically active ingredients comprise approximately 30% dealcoholized concentrated pomegranate wine, approximately 10% aqueous extract of pomegranate pericarp, and approximately 60% seed cake extract.

According to one aspect of the present invention there is provided a novel selective estrogen receptor modulator (SERM) derived from pomegranate. The novel SERM includes at least one item selected from the group consisting of: (a) a pomegranate seed product; (b) a pomegranate juice product and (c) a pomegranate peel product.

According to another aspect of the present invention there is provided a method for selectively modulating an estrogen receptor. The method includes the steps of providing at least one item selected from the group consisting of a pomegranate seed product, a pomegranate juice product and a pomegranate peel product and allowing a SERM contained in the at least one item to selectively modulate an estrogen receptor.

According to yet another aspect of the present invention there is provided a novel method of producing 17 alpha estradiol, the method includes the steps of: (a) extracting oil from pomegranate seeds; and (b) purifying the 17 alpha estradiol therefrom.

According to still another aspect of the present invention there is provided a novel method of producing gamma tocopherol, the method includes the steps of: (a) extracting oil from pomegranate seeds; and (b) purifying the gamma tocopherol therefrom.

According to an additional aspect of the present invention there is provided a novel method of producing campesterol, the method includes the steps of: (a) extracting oil from pomegranate seeds; and (b) purifying the campesterol therefrom.

According to yet additional aspect of the present invention there is provided a novel method of producing a composition containing a physiologically active amount of 17 alpha estradiol, the method includes the step of extracting oil from pomegranate seeds, the oil containing the physiologically active amount of 17 alpha estradiol.

According to still additional aspect of the present invention there is provided a novel method of producing a composition containing a physiologically active amount of gamma tocopherol, the method includes the step of extracting oil from pomegranate seeds; the oil containing the physiologically active amount of gamma tocopherol.

According to another aspect of the present invention there is provided a novel method of producing a composition containing a physiologically active amount of campesterol, the method includes the step extracting oil from pomegranate seeds, the oil containing the physiologically active amount of campesterol.

According to yet another aspect of the present invention there is provided a novel method for use of pomegranate seed oil, the method includes the step of administering the pomegranate seed oil to a subject to exert an effect on a physiologic process.

According to still another aspect of the present invention there is provided an improved method of retarding the process of atherosclerosis, the method includes the steps of: (a) fermenting juice extracted from pomegranates; and (b) administering the fermented juice to a patient.

According to an additional aspect of the present invention there is provided a composition for inhibiting cellular aging, the composition includes an effective amount of a pomegranate product.

According to further features in preferred embodiments of the invention described below, the pomegranate seed product is the result of a process selected from the group consisting of cold pressing, expeller pressing, supercritical fluid extraction with carbon dioxide, and lyophilization.

According to still further features in the described preferred embodiments the pomegranate seed product includes at least one item selected from the group consisting of pomegranate seed oil, an unsaponified fraction thereof and an alcohol extract of pomegranate seed cake.

According to still further features in the described preferred embodiments the alcohol employed to produce the alcohol extract includes at least one item selected from the group consisting of methanol, ethanol, propanol and butanol.

According to still further features in the described preferred embodiments the pomegranate juice product includes at least one item selected from the group consisting of pomegranate juice, fermented pomegranate juice, dried pomegranate juice, dried fermented pomegranate juice, partially fermented pomegranate juice, partially dried pomegranate juice, partially fermented partially dried pomegranate juice, reduced pomegranate juice, partially reduced pomegranate juice and lyophylysates and supercritical fluid extracts thereof.

According to still further features in the described preferred embodiments the mixture is provided in a form selected from the group consisting of a liquid, a powder, granules, a tablet, a capsule, a gel-cap, an ointment, a cream, a chewing gum, a food, a candy, an emulsion and a suppository.

According to still further features in the described preferred embodiments the mixture has utility for a purpose selected from the group consisting of retardation of the development of cancer, cancer treatment, hormone replacement therapy, retarding the aging process, skin treatment, relief of menopausal dryness, and relief of dyspareunia.

According to still further features in the described preferred embodiments the cancer is a hormone dependent cancer.

According to still further features in the described preferred embodiments the pomegranate peel product is selected from the group consisting of pomegranate peel residue present in pomegranate juice as a result of a juicing process, an aqueous extract of pomegranate peel, an alcohol extract of pomegranate peel, an extract performed with an organic solvent which is not an alcohol, and a supercritical fluid extract of pomegranate peel.

The supercritical fluid extract may be conducted with, for example, CO₂, ethanol, methanol, water, or combinations thereof.

According to still further features in the described preferred embodiments the method further includes the step of combining at least two of the at least one item.

According to still further features in the described preferred embodiments the method further includes the step of combining at least three of the at least one item.

According to still further features in the described preferred embodiments the step of providing the pomegranate juice product includes at least one process selected from the group consisting of extracting juice from a pomegranate, fermenting pomegranate juice, drying pomegranate juice, drying fermented pomegranate juice, partially fermenting pomegranate juice, partially drying pomegranate juice, partially fermenting partially dried pomegranate juice, reducing pomegranate juice, partially reducing pomegranate juice and lyophylysation of a product of any process belonging to the group.

According to still further features in the described preferred embodiments the subject is a selected from the group consisting of an animal and a human.

According to still further features in the described preferred embodiments the physiologic process is selected from the group consisting of aging, apoptosis, prostaglandin biosynthesis, estrogen activity, development of elasticity, development of tone, humectification, formation of age spots, crosslinking of collagen, oxidation of LDL and telomerase activity.

According to still further features in the described preferred embodiments the effect is selected from the group consisting of retarding aging, retarding apoptosis, inhibition of prostaglandin biosynthesis, agonism of estrogen activity, increasing elasticity, increasing tone, increasing humectification, dissipating age spots, retarding the process of collagen crosslinking, retarding LDL oxidation and stimulating telomerase activity.

According to still further features in the described preferred embodiments the step of administering is conducted in a manner selected from the group consisting of locally and systemically.

According to still further features in the described preferred embodiments the step of administering locally includes administration via an administration route selected from the group consisting of topical application to skin, intravaginal application, intrarectal application, intranasal application, intraocular application and inhalation.

According to still further features in the described preferred embodiments the step of administering systemically includes administration via an administration route selected from the group consisting of oral administration, injection, subcutaneous implantation, intravaginal application, intrarectal application, and inhalation.

According to still further features in the described preferred embodiments the pomegranate seed oil is administered together with at least one additional item selected from the group consisting of a pomegranate juice product and a pomegranate peel product.

According to still further features in the described preferred embodiments the effect includes an antioxidant effect.

According to still further features in the described preferred embodiments the antioxidant effect is at least partially produced by at least one item selected from the group consisting of 17 alpha estradiol, gamma tocopherol and campesterol.

According to still further features in the described preferred embodiments the juice further contains material derived a pomegranate peel product.

According to still further features in the described preferred embodiments the pomegranate peel product includes residual pomegranate peel present in the juice when extracted from the pomegranates.

According to still further features in the described preferred embodiments the method includes the additional step of adding pomegranate peel to the juice.

According to still further features in the described preferred embodiments the step of fermenting serves to at least partially de-glycosylate flavonoids in the juice, thereby enhancing their bio-availability.

According to still further features in the described preferred embodiments the method includes the additional step of further administering a pomegranate seed product with the fermented juice.

According to still further features in the described preferred embodiments the pomegranate seed product includes at least one item selected from pomegranate seed oil and an unsaponified fraction thereof.

According to still further features in the described preferred embodiments the pomegranate product includes at least one item selected from the group consisting of a pomegranate seed product, a pomegranate juice product and a pomegranate peel product.

According to still further features in the described preferred embodiments pomegranate products, especially pomegranate seed and derivatives thereof, are prepared from Wonderful cultivar pomegranates. More preferably, these pomegranates are organically grown, still more preferably, they are grown at Kibbutz Sde Eliahu in Israel.

The present invention successfully addresses the shortcomings of the presently known configurations by providing a mixture of a pomegranate seed oil product and a pomegranate juice product, and pharmaceutical compositions containing same, which has potential efficacy in prevention or treatment of cancer and other medical conditions. Further, the present invention successfully addresses the shortcomings of the presently known configurations by providing a variety of pomegranate products useful in improving health and methods of use thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a histogram illustrating estrogenic activity of pomegranate juice (PJ) in a yeast estrogen screen;

FIG. 2 is a is a histogram illustrating aromatase inhibition by polyphenols originating in seed oil, peel extract and wine of pomegranates;

FIG. 3 is a table providing a numerical summary of the data of FIG. 2;

FIG. 4 is a histogram illustrating estrogenic activity of pomegranate non-saponifiable fraction (NSF) in a yeast estrogen screen;

FIG. 5 is a graph illustrating the effect of polyphenol fractions from pomegranate seed oil, pericarp and fermented and fresh juice on proliferation of estrogen-dependent human breast cancer cells (MCF-7) in vitro;

FIG. 6 is a graph illustrating the effect of effect of polyphenol fractions from pomegranate seed oil, pericarp, and fermented and fresh juice on proliferation of estrogen-independent human breast cancer cells (MDA-MB-231) in vitro;

FIG. 7 is a graph illustrating the effect of pomegranate pericarp polyphenols on the proliferation of selected cancerous and normal cell lines. Normal cell lines: Human Umbilical Vein Endothelium [HUVE] and Human Foreskin Keratinocytes [HFK]). Cancerous lines murine melanocytic melanocytes [B16], human lung carcinoma [A549], human T cell leukemia [CCRF-HSB-2], human gastric lymph node metastasis [TGBC11TKB];

FIG. 8 is a is a graph illustrating the effect of pomegranate fermented juice polyphenols on the proliferation of selected cancerous and normal cell lines (cell lines as detailed for FIG. 8);

FIG. 9 is a graph of the effect of pomegranate fresh juice polyphenols on the proliferation of selected cancerous and normal cell lines (cell lines as detailed for FIG. 8). The effect here is clearly milder than for the fermented juice. Again, the most sensitive lines are the metastatic gastric and the leukemia. The normal cell lines are not affected;

FIG. 10 is a graph of the effect of pomegranate pericarp polyphenols on differentiation of HL-60 human leukemia cells. The Y axis is proliferation, the absolute amount of cell growth. Cellular differentiation is observed most strongly in two of the assays for cell differentiation, namely nitro blue tetrazolium reducing activity and non-specific esterase activity. Less strong results were observed using the phagocytic activity and specific esterase indices {NBT Nitro blue tetrazolium reducing activity; NSE Non-specific esterase activity; SE Specific esterase activity; PG Phagocytic activity; PR Cellular proliferation);

FIG. 11 is a graph of the effect of pomegranate fermented juice polyphenols on differentiation of HL-60 human leukemia cells (assays as detailed for FIG. 11);

FIG. 12 is a graph of the effect of pomegranate fresh juice polyphenols on differentiation of HL-60 human leukemia cells (assays as detailed for FIG. 11);

FIG. 13 is a histogram of the effect of pomegranate seed oil on invasion (metastasis) of MCF-7 human estrogen-dependent breast cancer cells in vitro;

FIGS. 14-18 are graphs of the effect of pure pomegranate seed oil on proliferation of human estrogen-dependent human breast cancer cells (MCF-7) in vitro (at 24, 72, 120, 168 and 26 hours of growth respectively);

FIG. 19 is a graph of the effect of pure pomegranate seed oil on proliferation of human LNCaP prostate cancer cells in vitro (at 120 hours);

FIG. 20 is a histogram illustrating aromatase inhibition by polyphenol fractions derived from seed oil, pericarp and fermented juice of pomegranate;

FIG. 21 is a histogram illustrating the effect of Pomegranate Fermented Juice Polyphenols on proliferation of hPCPs (stromal benign prostatic hypertrophy) cells

FIG. 22 is a is a histogram illustrating the effect of pomegranate seed oil polyphenols on proliferation of hPCPs (stromal benign prostatic hypertrophy) cells;

FIG. 23 is a histogram illustrating the effect of combination of pomegranate fermented juice and seed oil polyphenols on proliferation of hPCPs (stromal benign prostatic hypertrophy) cells;

FIG. 24 is a histogram illustrating the effect of pomegranate fermented juice polyphenols (W) on proliferation of human LNCaP human prostate cancer cells;

FIG. 25 is a graph of inhibition of proliferation of PC-3 human prostate cancer cells at progressively higher concentrations. Ethox=ethanol control, W=pomegranate fermented juice polyphenols, P=pomegranate pericarp polyphenols, SCFO=pure pomegranate seed oil;

FIG. 26 is a histogram of an alternative portrayal of inhibition of proliferation of PC-3 human prostate cancer cells by pomegranate fractions. Ethox=ethanol control, W=pomegranate fermented juice polyphenols, P=pomegranate pericarp polyphenols, SCFO=pure pomegranate seed oil;

FIG. 27 is a graph illustrating the effect of pomegranate pericarp extract polyphenol fraction of proliferation on poorly differentiation, androgen-independent PC-3 human prostate cancer cells in vitro. The inhibition is concentration-dependent;

FIG. 28 is a graph illustrating the effect of pomegranate fermented juice polyphenol fraction (W) on the proliferation of PC-3 poorly differentiated, androgen-independent human prostate cancer cells in vitro. A concentration-dependent inhibition is observed;

FIG. 29 is a graph illustrating the effect of effect of pure pomegranate seed oil on proliferation of human PC-3 poorly differentiated androgen-independent prostate cancer cells in vitro;

FIG. 30 is a graph illustrating the concentration-dependent inhibition of poorly differentiation LNCaP human androgen-independent prostate cancer cells in vitro by pomegranate pericarp polyphenol fraction;

FIG. 31 is a graph illustrating the effect on proliferation of very poorly differentiated androgen-independent DU-145 human prostate cancer cells in vitro of pomegranate pericarp polyphenol fraction. The inhibition is concentration-dependent;

FIG. 32 is a graph illustrating the effect of a polyphenol fraction of pomegranate fermented juice on the proliferation of very poorly differentiated human androgen-independent DU-145 prostate cancer cells in vitro. A concentration-dependent inhibition is observed;

FIG. 33 is a graph illustrating the effect of pure pomegranate seed oil on the proliferation of human very poorly differentiated androgen-independent DU-145 human prostate cancer cells in vitro. Inhibition is noted at the highest concentration tested;

FIG. 34 is a histogram illustrating the effect of pomegranate fermented juice and pericarp polyphenols on the GI stage of the cell cycle in B16 melanin pigment producing mouse melanoma cells. The y axis denotes % of cells at G1 phase at time of measurement. Increasing concentrations of the pomegranate fractions result in an increased number of cells at the G1 phase of arrest;

FIG. 35 is a histogram illustrating the effect of pomegranate fermented juice and pericarp polyphenols on the G2 stage of B16 melanin pigment producing mouse melanoma cells. The y axis denotes % of cells at G2 at time of measurement. Increasing concentrations of the pomegranate fractions result in an increased number of cells at the G2 stage of arrest;

FIG. 36 is a histogram illustrating the effect of pomegranate fermented juice and pericarp polyphenols on the S stage of the cell cycle in B16 melanin pigment producing mouse melanoma cells. The y axis denotes % of cells at stage S (synthesis of DNA) of cell cycle at time of measurement. Increasing concentrations of the pomegranate fractions result in decreased DNA synthesis;

FIG. 37 is a histogram summarizing overall effect on cell growth (proliferation) of B-16 murine melanin pigment producing melanoma cells by pomegranate fermented juice and pericarp polyphenol fractions. Increasing concentrations of the active materials result in an overall decrease in the growth of the cells;

FIG. 38 is a histogram illustrating the effect on growth of HL-60 human leukemia cells in vitro by selected fractions of pomegranate fruit. [FJP=fermented pomegranate juice polyphenol fraction; OP=pomegranate seed polyphenol fraction. EtOH (ethanol) control used here is at a much higher concentration that used for dissolving seed oil. OP and FJP are dissolved in DMSO (dimethyl sulfoxide). Powerful inhibition is caused by FJP, by fermented juice and by pomegranate seed oil at increasing concentrations;

FIG. 39 is a histogram illustrating the effect of selected pomegranate fruit fractions on the G1 phase of the cell cycle in HL-60 human leukemia cells. [fractions as in FIG. 38] Increasing the dose of the pomegranate fractions increases the percentage of cells at the GI stage of arrest. Seed oil dissolved in 20 microliters per ml ethanol; all other fractions in DMSO 12.5 microliters per ml;

FIG. 40 is a histogram illustrating the effect of pomegranate fruit fractions on the G2 stage of cell division in human HL-60 promyelocytic leukemia cells. Only the fermented juice polyphenols appear to have a significant effect in prolonging this stage;

FIG. 41 is a histogram illustrating the effect of selected pomegranate fruit fractions on the S phase of the cell cycle in HL-60 human promyelocytic leukemia cells. [fractions as in FIG. 38] The fermented juice polyphenol fraction completely eliminates this phase. A similar, though attenuated, effect is observed for the simple concentrated fermented juice, as expected. The seed oil does not have this effect;

FIG. 42 is a histogram illustrating apoptosis in HL-60 human leukemia cells in vitro induced by selected pomegranate fruit fractions. [fractions as in FIG. 38] The highest degree of apoptosis is observed for the whole pomegranate seed oil. Solvent for the OP, FJP and fermented juice is DMSO 12.5 microliters per ml. The seed oil is dissolved in ethanol. At the lower concentration of seed oil, the ethanol concentration is 10 micrograms per ml. At the higher concentration, the ethanol concentration is 20 micrograms per ml;

FIG. 43 is a histogram illustrating inhibition of 17-beta-hydroxysteroid dehydrogenase Type 1 by selected pomegranate fractions. [P=pericarp extract, W=fermented juice extract, SCFO=pomegranate seed oil extracted with supercritical CO₂, EM-251=positive control (16-alpha-bromopropyl-estradiol)];

FIG. 44 is a graph of the effect of pomegranate fermented juice extract (W) on proliferation of human multiple myeloma cell line HS-Sultan (HSS);

FIG. 45 is a graph of the effect of pomegranate fermented juice extract (W) and pomegranate pericarp extract (P) on proliferation of human multiple myeloma cell line MM.1S;

FIG. 46 is a graph of the effect of pomegranate fractions on proliferation of human multiple myeloma cell line U266. [W=fermented juice extract, P=pericarp extract, SESCO=supercritical CO₂ extracted seed oil, SEEE=ethanolic extract of seed cake (following oil extrusion)];

FIG. 47 is a histogram comparing pomegranate fermented juice extract (W) to a known Vitamin D differentiation inducing agent (cont-D) with respect to the prevention of carcinogenesis in a murine mammary gland organ culture;

FIG. 48 is a histogram illustrating dose-dependent inhibition of HT-29 human colon adenocarcinoma cells by pomegranate fermented juice (W) and pericarp (P) extracts;

FIG. 49 is a histogram illustrating dose-dependent inhibition of proliferation of rapidly dividing WI38 human diploid normal embryonic lung tissue by pomegranate fermented juice (W) and pericarp (P);

FIG. 50 is a histogram illustrating dose-dependent inhibition on HPB-ALL human thymoma cells of pomegranate fermented juice (W) and pericarp (P) extracts relative to quercetin (Q);

FIG. 51 is a histogram illustrating a comparison of the anti-proliferative effect of pomegranate pericarp (P) and fermented juice (W) extracts on human thymoma cells (HPB-ALL) and their normal conterparts (PBL);

FIG. 52 is a flow diagram showing production steps in manufacture of 1000 doses of an elixir for women according to the present invention;

FIG. 53 is a flow diagram showing production steps in manufacture of 1000 doses of an elixir for men according to the present invention;

FIG. 54 is a histogram of estrogen alpha activity of various pomegranate fractions at different concentrations. Blue bars represent 1 mcg/ml, red bars represent 10 mcg/ml and yellow bars represent 100 mcg/ml. W indicates fermented pomegranate juice polyphenols; P indicates pomegranate pericarp polyphenols; SEEE indicates Sde Eliahu ethanolic pomegranate seed extract; SEME indicates Sde Eliahu methanolic pomegranate seed extract; TEE indicates Turkish ethanolic pomegranate seed extract; TME indicates Turkish methanolic pomegranate seed extract; MeOH indicates methanol; EtOH indicates ethanol; TPO indicates Turkish pressed oil; SEPO indicates Sde Eliahu pressed oil; SESCO indicates Sde Eliahu supercritical carbon dioxide extracted oil; and F indicates pomegranate flower ethanolic extract.

FIG. 55 is a histogram of estrogen beta activity of various pomegranate fractions at different concentrations. Concentrations and fractions are as in FIG. 54;

FIG. 56 is graph of % apoptosis as a function of concentration in a cell culture assay pomegranate test oil is indicated by diamonds and alpha tocopherol control is indicated by squares;

FIG. 57 is a graph of absorbance at 234 nanometers as a function of time. Kontrolle indicates olive oil; #25 indicates Sde Eliahu pressed oil; #26 indicates a CO2 supercritical fluid extract of Sde Eliahu pressed oil and # 27 indicates Turkish pressed oil;

FIG. 58 is a graph of absorbance at 234 nanometers as a function of time. Various concentrations of #26 (CO2 supercritical fluid extract of Sde Eliahu pressed oil) were employed;

FIG. 59 is a graph of absorbance at 234 nanometers as a function of time. W indicates (LDL) of pomegranate fermented juice polyphenol fraction, N indicates unsaponifiable fraction of pomegranate seed oil.

FIGS. 60a, b, and c illustrate the molecular structures of luteolin, quercetin, and kaempferol respectively.

FIG. 61 is a flow scheme of on-line characterization screening of pomegranate peel extract. (1, Biogradient; 2, Flow split 50 μl/min to biochemical detection and 1150 μl/min to MS; 3, Addition of ER beta via superloop; 4, Reaction coil; 5, Addition of coumestrol; 6, Reaction coil; 7, Restricted access column; 8, fluorescence detector)

FIG. 62 depicts an analysis of acid hydrolyzed pomegranate peel extract. Top trace indicates total ion current full scan MS as a function of time. Bottom trace indicates biochemical detection readout of the same sample. Top and bottom traces are corrected for time delay.

FIGS. 63a and b depict MS and MS/MS spectra corresponding to the first peak in the bottom trace of FIG. 62. A) MS and MS/MS spectra of co-eluting compound m/z 285.5. B) MS and MS/MS spectra of co-eluting compound m/z 301.5). Time difference between MS spectra A and B equals 0.3 min.

FIG. 64 depicts MS and MS/MS spectra, corresponding to the second peak in the biochemical trace, of compound with m/z 285.5.

FIGS. 65A and b 65 illustrate the correlation of bioactivity to reference compounds: luteolin (1), quercetin (2) and kaempferol (3). A) MS ion current trace of hydrolyzed pomegranate peel extract (m/z 285.3 and 301.5). B) top trace: MS ion current trace of reference solution: 260 μM luteolin, 280 μM quercetin and 460 μM kaempferol (m/z 285.3 and 301.5). bottom trace: Bioactivity trace of reference solution.

FIGS. 66a and b compare original pomegranate peel extract and acid hydrolyzed pomegranate peel extract. A) analysis original pomegranate peel extract. Top: MS/MS trace of (1) glycosylated luteolin m/z199.5. Bottom: biochemical trace. B) analysis acid hydrolyzed pomegranate peel extract. Top: MS trace (1) glycosylated luteolin (2) luteolin (m/z 285.5) (3) kaempferol (m/z 285.5). Bottom: biochemical trace.

FIGS. 67a, b and c constitute a characterization of glycosylated phytoestrogens in pomegranate peel extract. A) MS ion current for m/z 285.5 (kaempferol/luteolin). B) MS spectrum kaempferol-glucoside (Rₜ 6.74 min). C) MS spectrum kaempferol-rhamnoglucoside (Rₜ 7.19 min). MS/MS data dependent scanning, original extract. Gradient 0-20% in 20 min. Minimum/maximum flow rate in Biogradient respectively 0.37 and 0.5 ml/min.

### DESCRIPTION OF THE PREFERRED EMBODIMENTS

The present invention is of a mixture of a pomegranate seed oil product and a pomegranate juice product, and pharmaceutical compositions containing same which can be used to prevent or treat a variety of medical conditions.

Specifically, the present invention can be used to prevent or treat cancer, especially hormone dependent cancer.

The principles and operation of a mixture of a pomegranate seed oil product and a pomegranate juice product, and pharmaceutical compositions containing same according to the present invention may be better understood with reference to the drawings and accompanying descriptions.

For purposes of this specification and the accompanying claims, the terms “pericarp”, “rind” and “peel” are considered synonymous and are used interchangeably.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The present invention is of a cancer chemo-preventive mixture. The ability of mixtures according to the present invention to inhibit cancer stems from their ability to inhibit cellular proliferation and stimulate cellular differentiation as detailed hereinbelow in the examples section. These general cellular phenomena are explained by apparent influences on estrogen and aromatase activities as well as 17-beta-hydroxysteroid dehydrogenase Type 1 activity as detailed in examples hereinbelow. The mixture includes a pomegranate seed oil product and a pomegranate juice product. The pomegranate seed oil product may be, for example, the result of a process such as expeller pressing, supercritical fluid extraction with carbon dioxide, or lyophilization. The pomegranate seed oil product may be produced from a material including, but not limited to, pomegranate seeds and pomegranate seed cake. As such, the pomegranate seed oil product may be, for example, pomegranate seed oil or a non saponifiable fraction thereof.

The pomegranate juice product may include, for example pomegranate juice, fermented pomegranate juice, dried pomegranate juice, dried fermented pomegranate juice, partially fermented pomegranate juice, partially dried pomegranate juice, partially fermented partially dried pomegranate juice, reduced pomegranate juice, partially reduced pomegranate juice and lyophylysates thereof or any combination of these ingredients.

The present invention is further embodied by a pharmaceutical composition including physiologically active amounts of a pomegranate seed oil product and a pomegranate juice product as defined hereinabove. The pharmaceutical composition further includes a pharmaceutically acceptable carrier. In some cases, the mixture or pharmaceutical composition may further include a pomegranate peel product in order to increase efficacy thereof. The pomegranate peel product may be, for example the pomegranate peel residue present in pomegranate juice as a result of a juicing process, an aqueous extract of pomegranate peel, an alcohol extract of pomegranate peel, an extract performed with an organic solvent which is not alcohol, a supercritical CO₂ extract of pomegranate peel or any combination thereof. Preferably, the pomegranate peel product contains at least one item selected from the group consisting of quercetin, kaempferol, luteolin, derivatives thereof and combinations thereof.

The mixture or pharmaceutical composition may be provided in myriad forms, including but not limited to, a liquid, a powder, granules, a tablet, a capsule, a gel-cap, an ointment, a cream, a chewing gum, a food, a candy, an emulsion and a suppository.

Because of the demonstrable effect of the components of the mixture of the present invention on estrogen and on enzymes involved in estrogen synthesis, the present invention will most likely have special efficacy in treatment of cancer which is hormone dependent. Such hormone dependent cancers include, but are not limited to breast cancer and prostate cancer.

Alternately or additionally, the pharmaceutical composition of the present invention may be efficaciously employed for treatment of a medical condition including, but not limited to cancer, Alzheimer's disease, climacteria, benign prostatic hyperplasia and estrogen deficiency. Additional uses include, but are not limited to, retardation of the development of cancer, cancer treatment, hormone replacement therapy, retarding the aging process, skin treatment, relief of menopausal dryness, and relief of dyspareunia.

In vivo cell culture data on cell lines derived from cancers including breast cancer, prostate cancer, melanoma, human lung carcinoma, human T cell leukemia, human gastric lymph node metastasis and benign prostatic hypertrophy are presented in examples hereinbelow. The term “treatment”, as used in this specification and the accompanying claims is to be construed in its broadest possible sense. As such treatment includes, but is not limited to, prophylactic treatment, palliative treatment and therapeutic treatment.

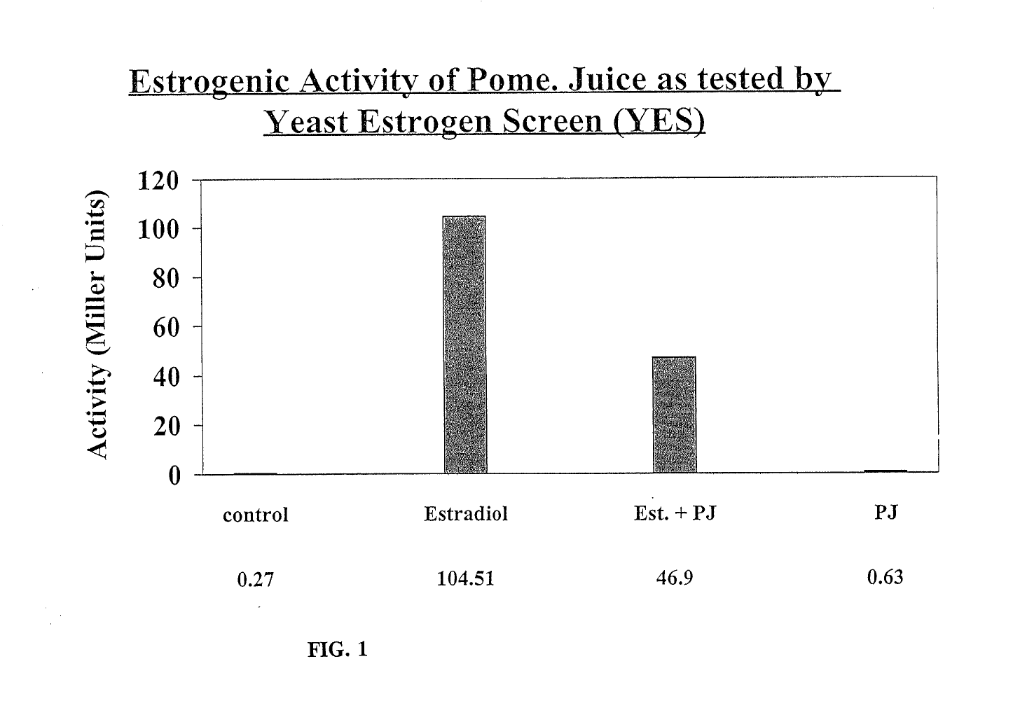

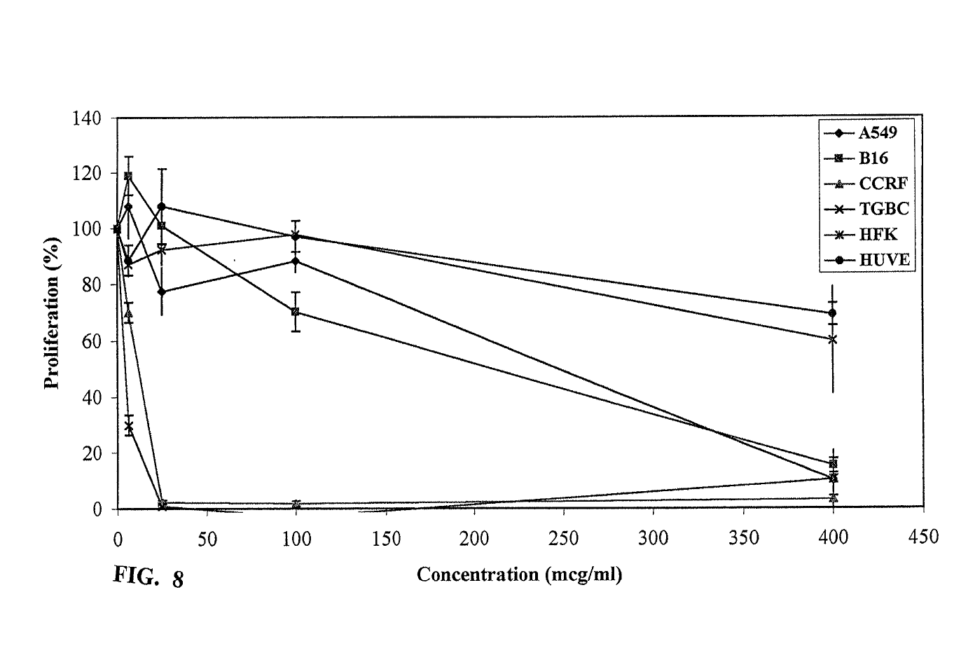

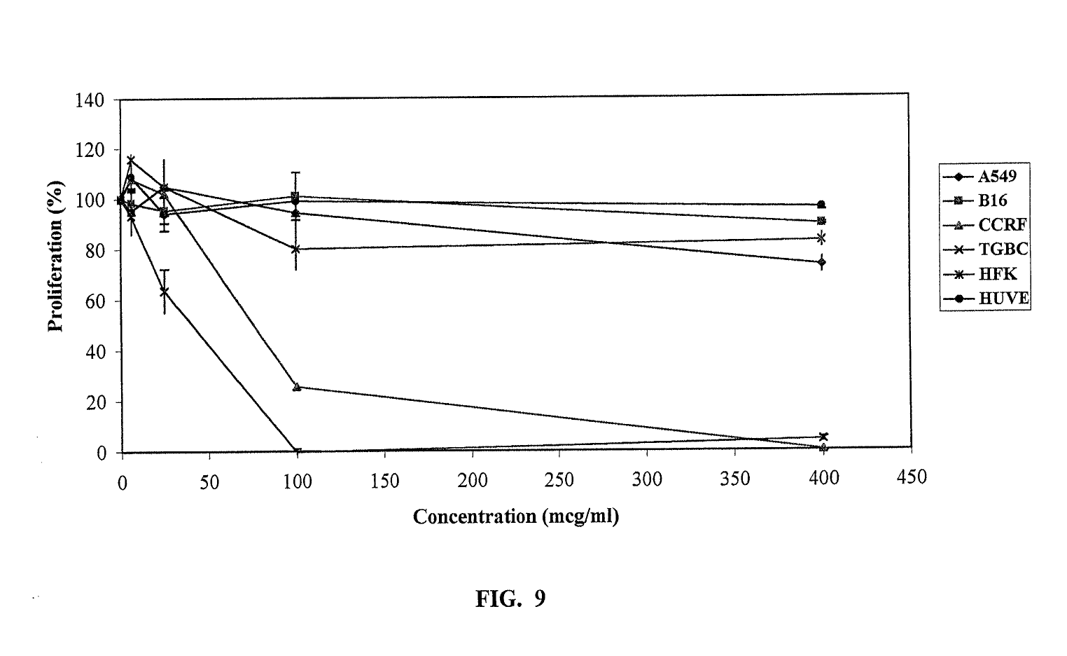

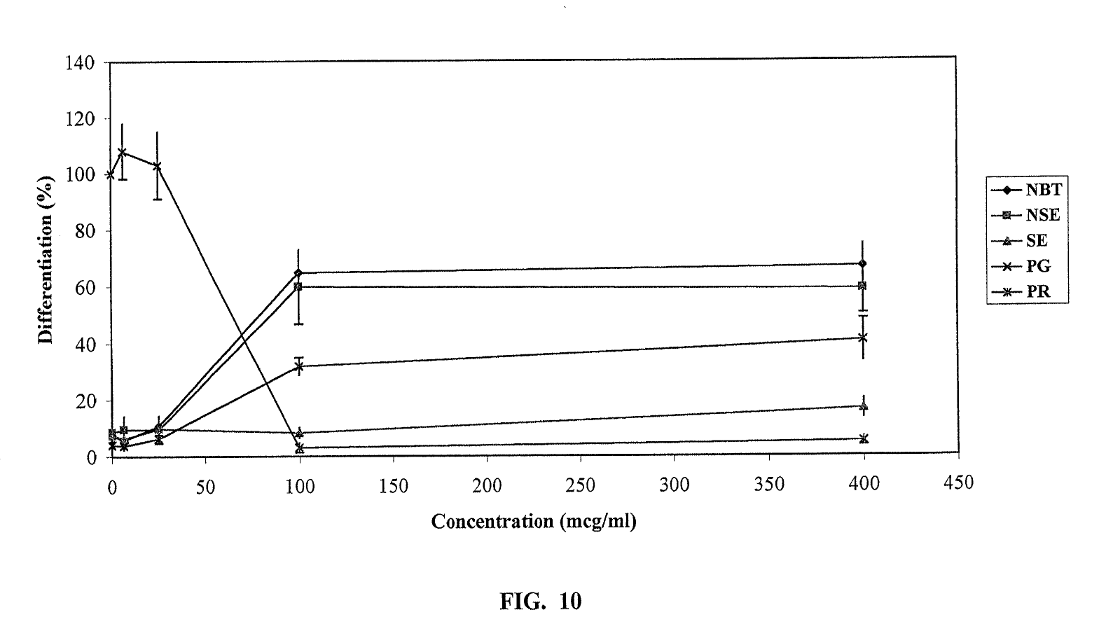

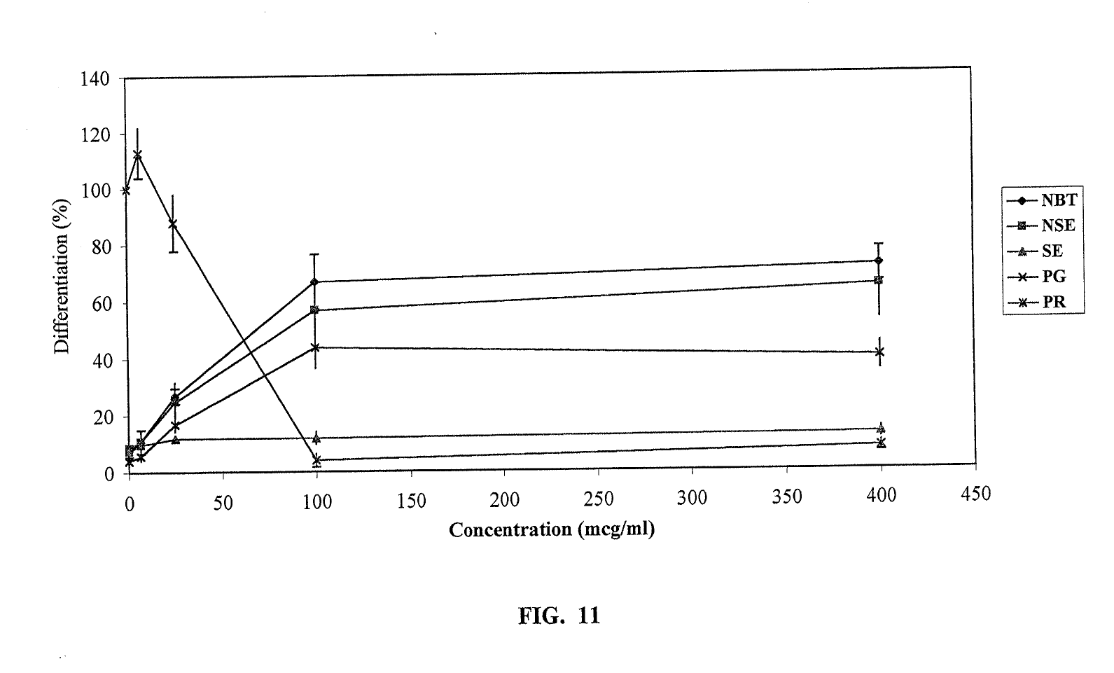

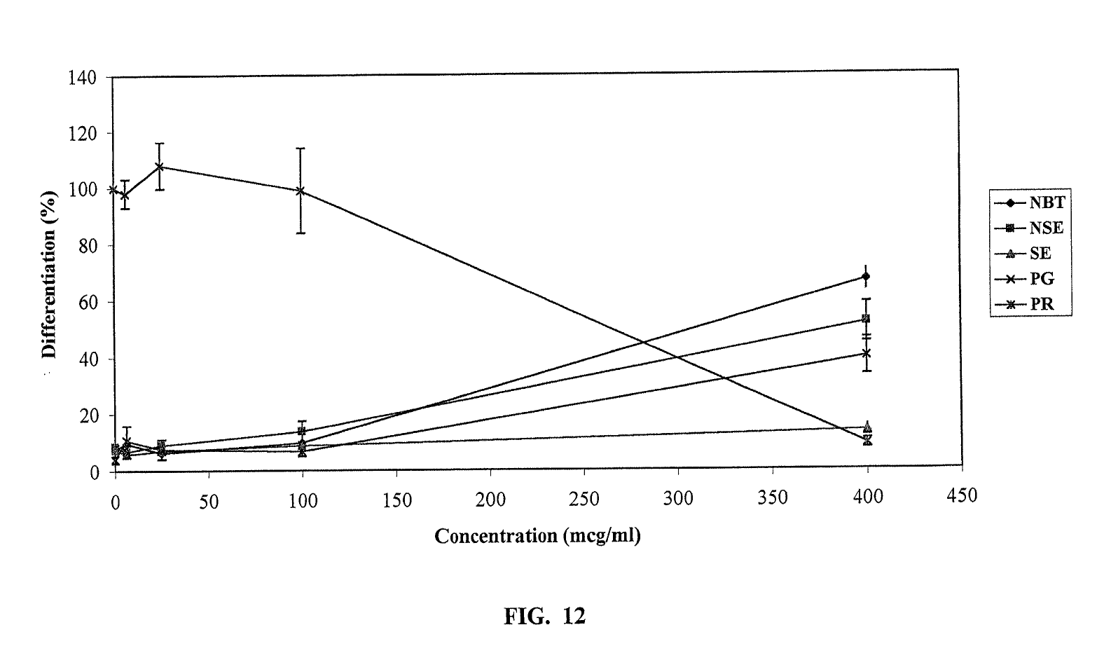

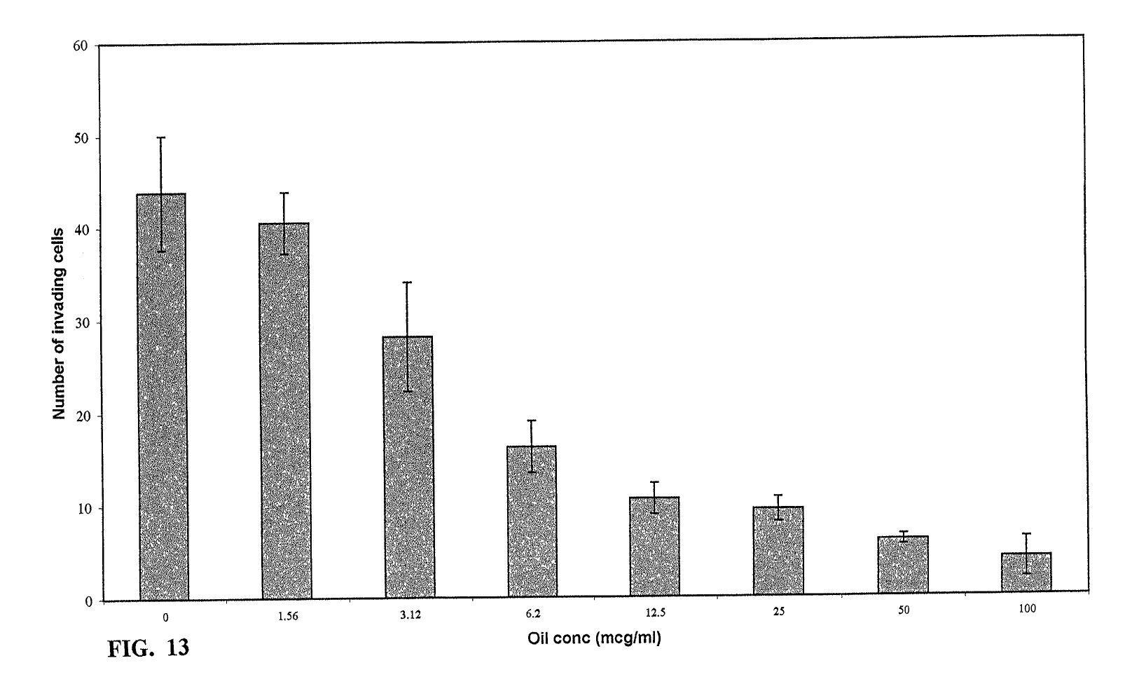

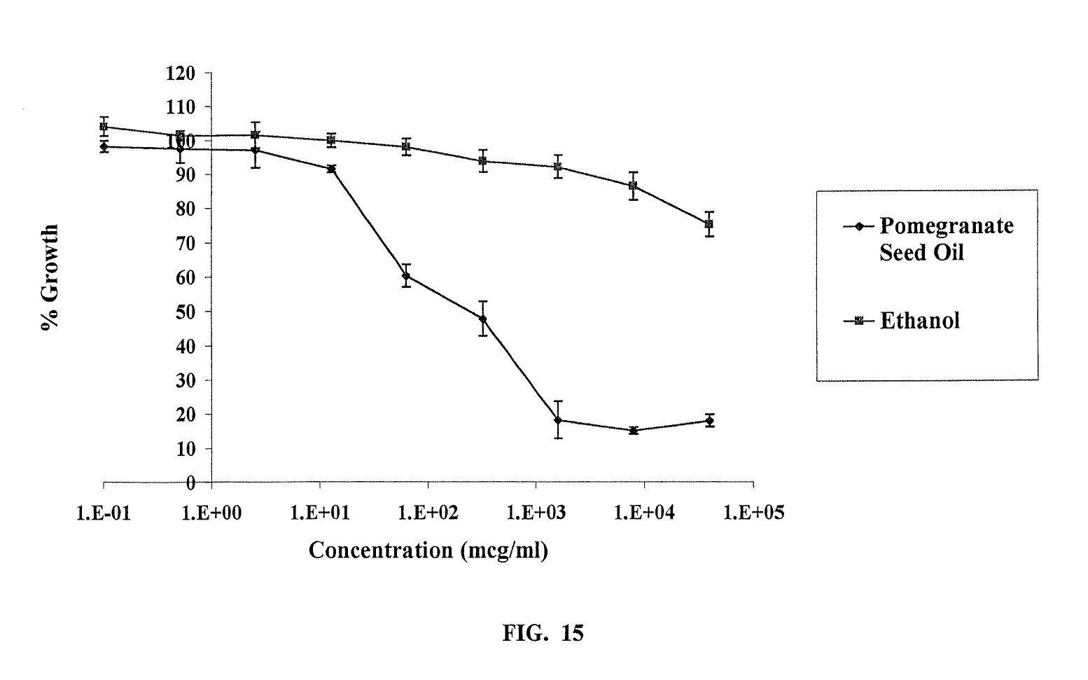

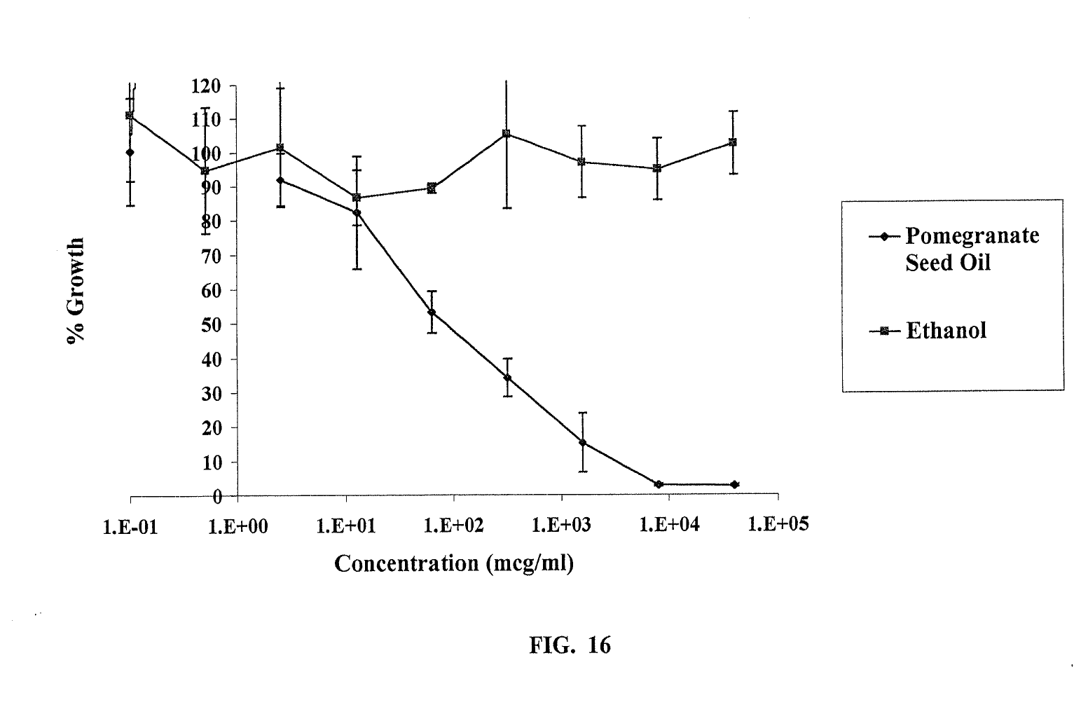

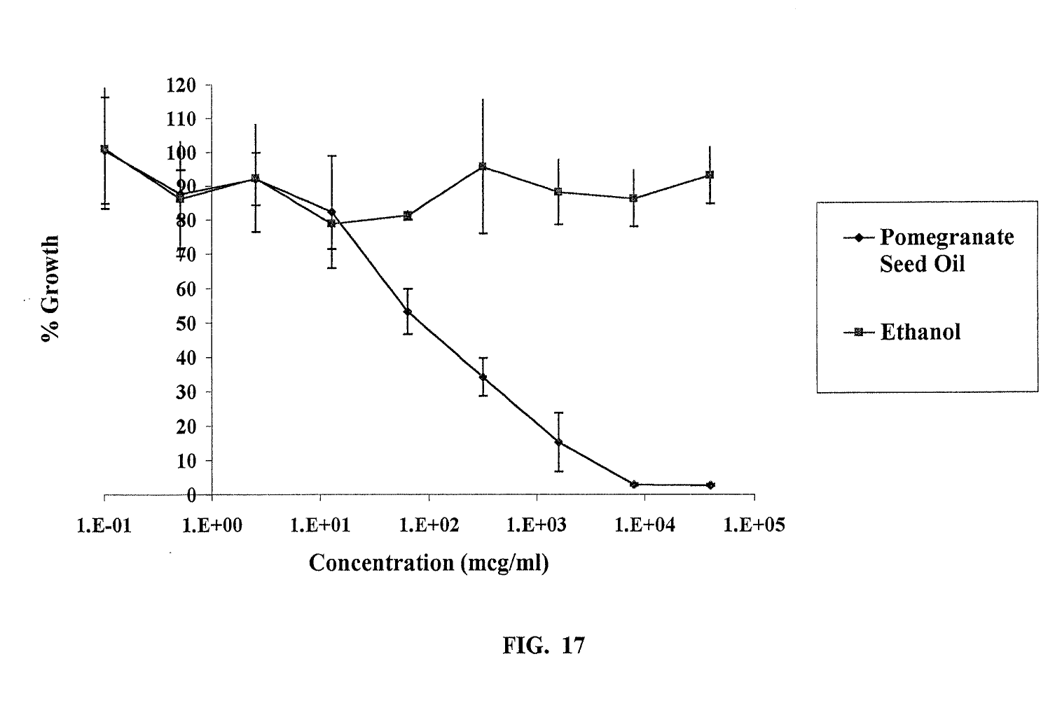

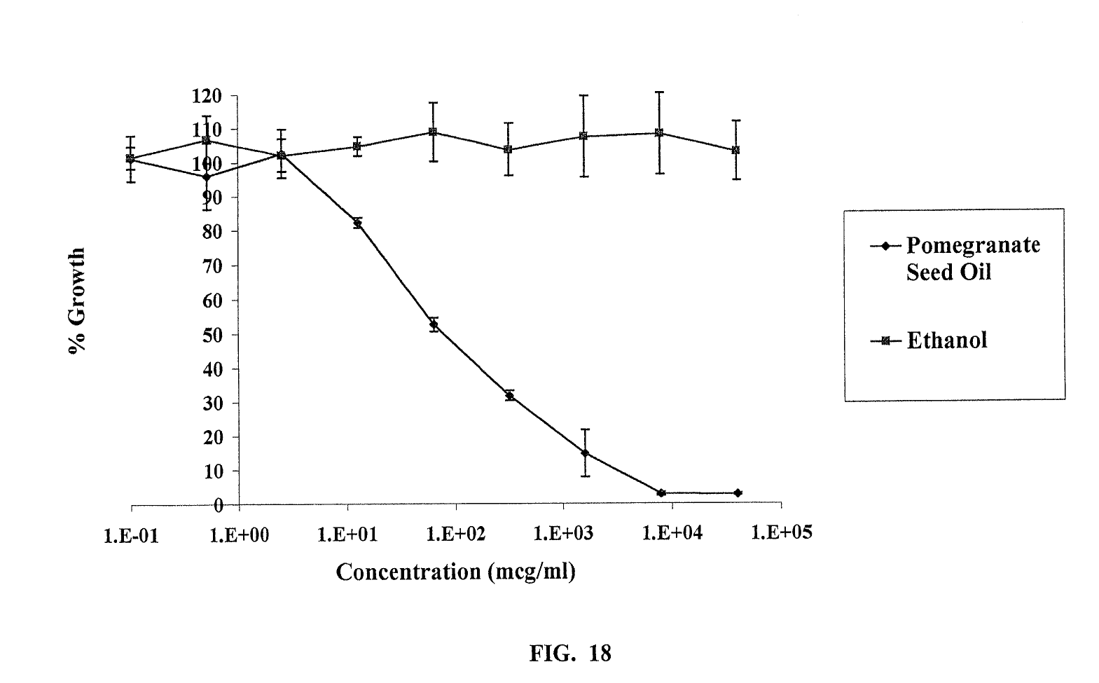

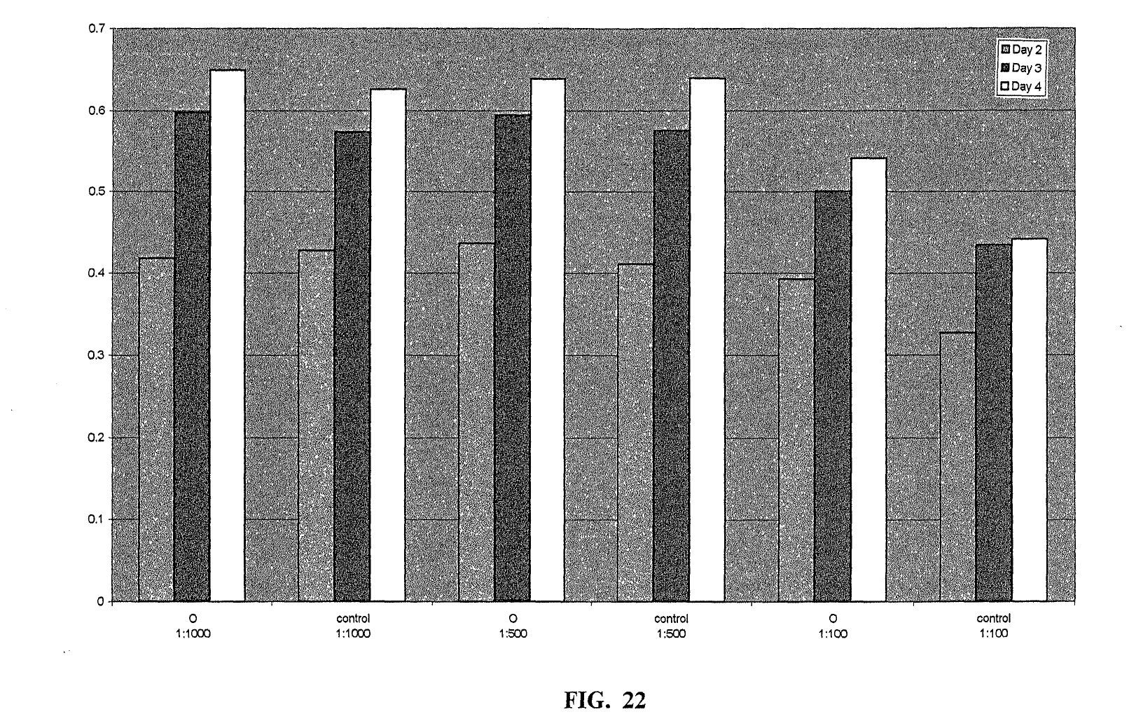

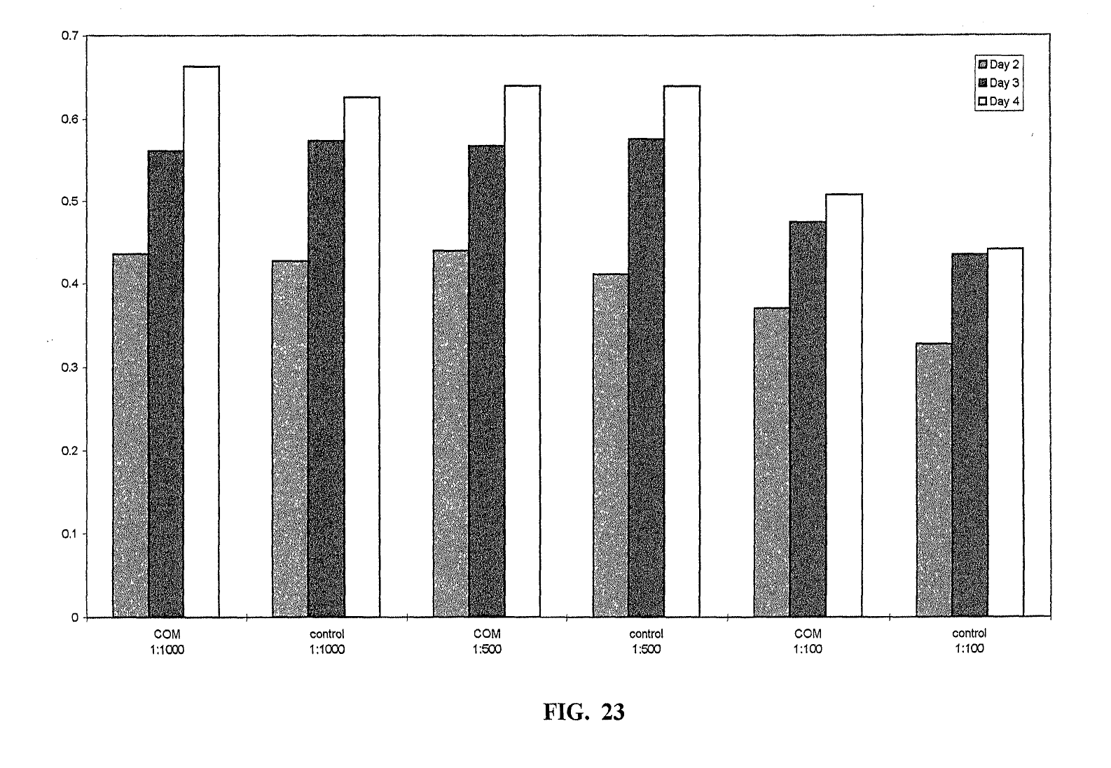

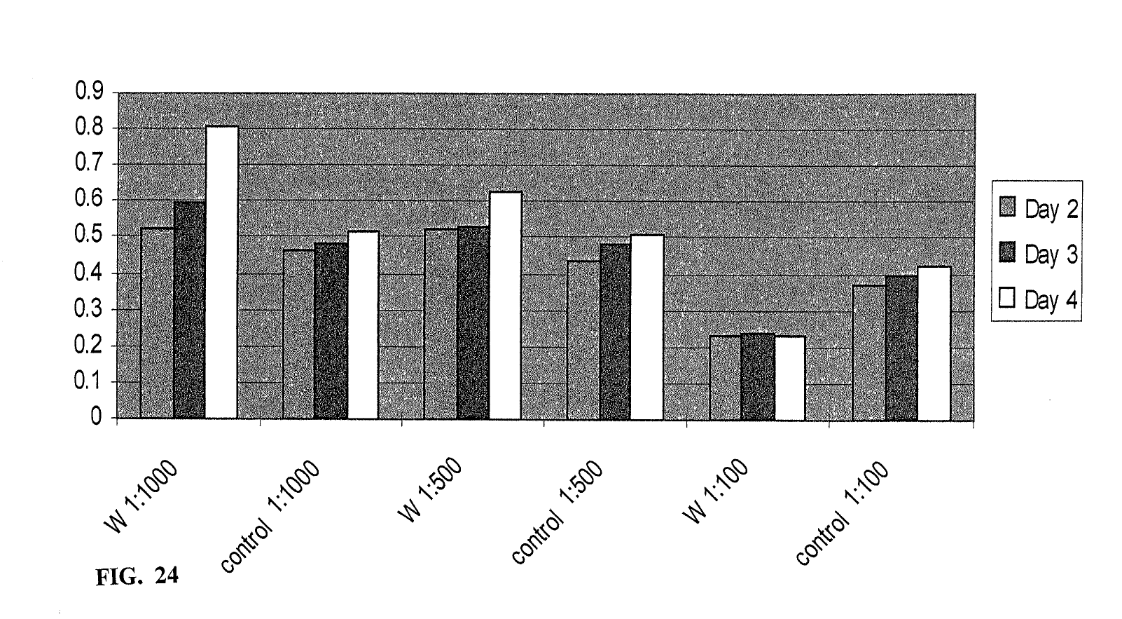

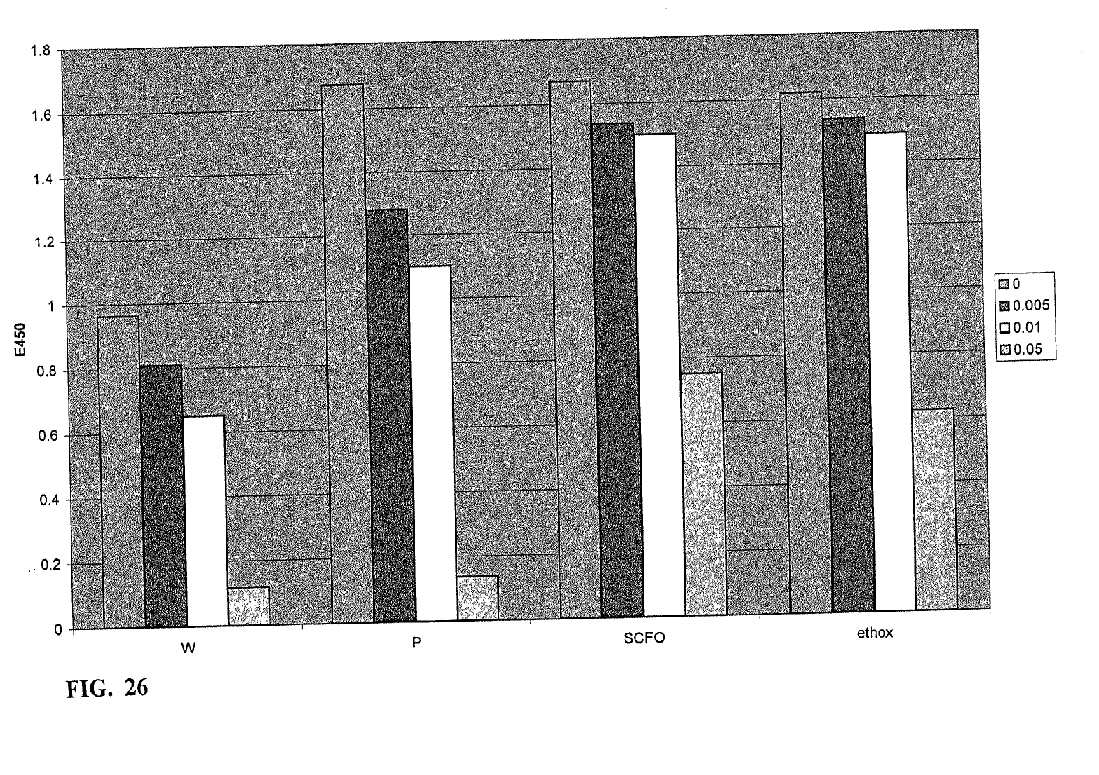

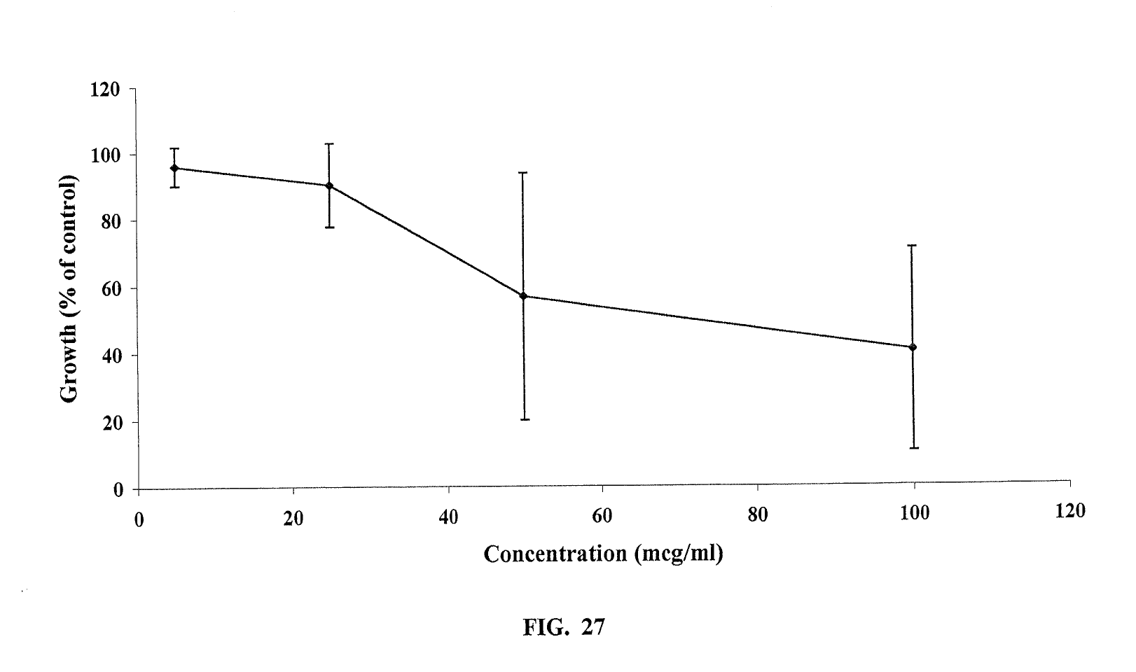

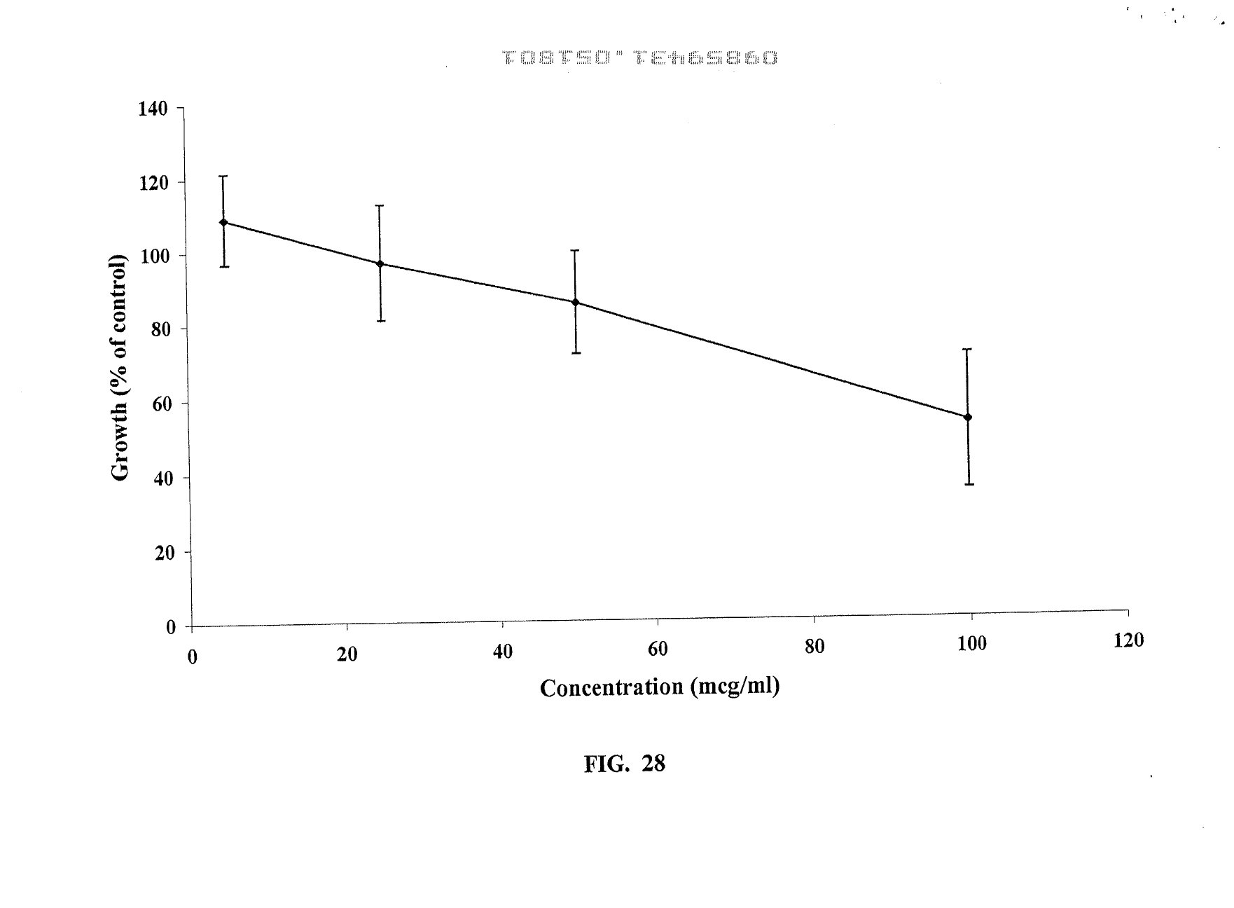

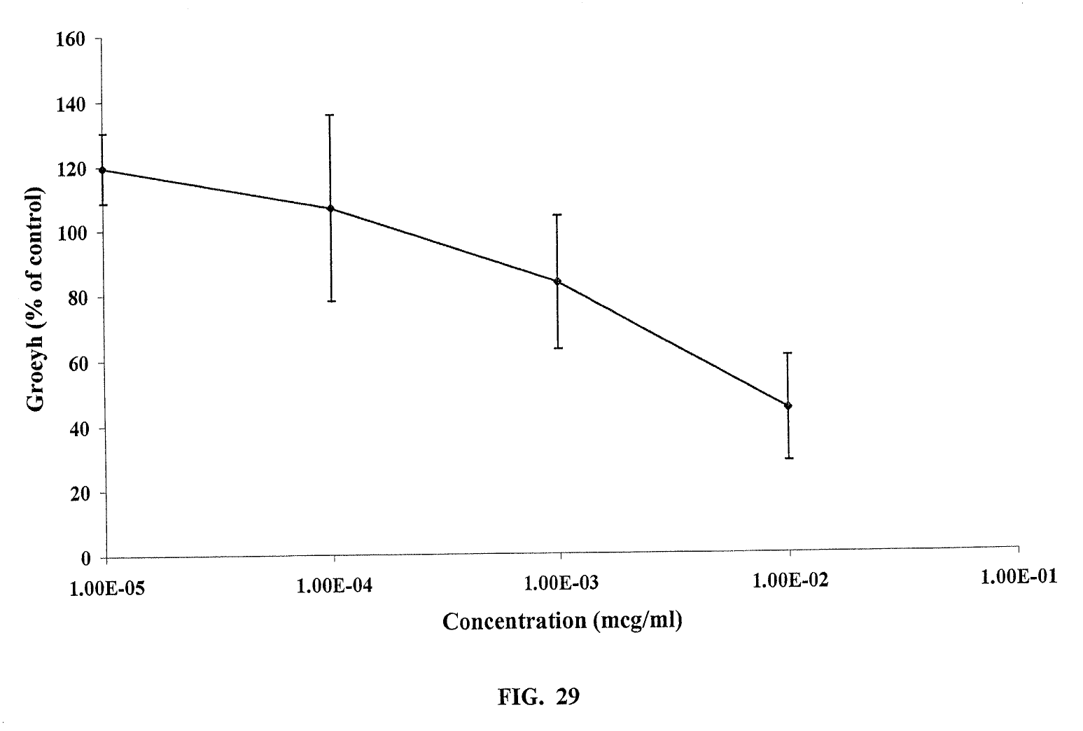

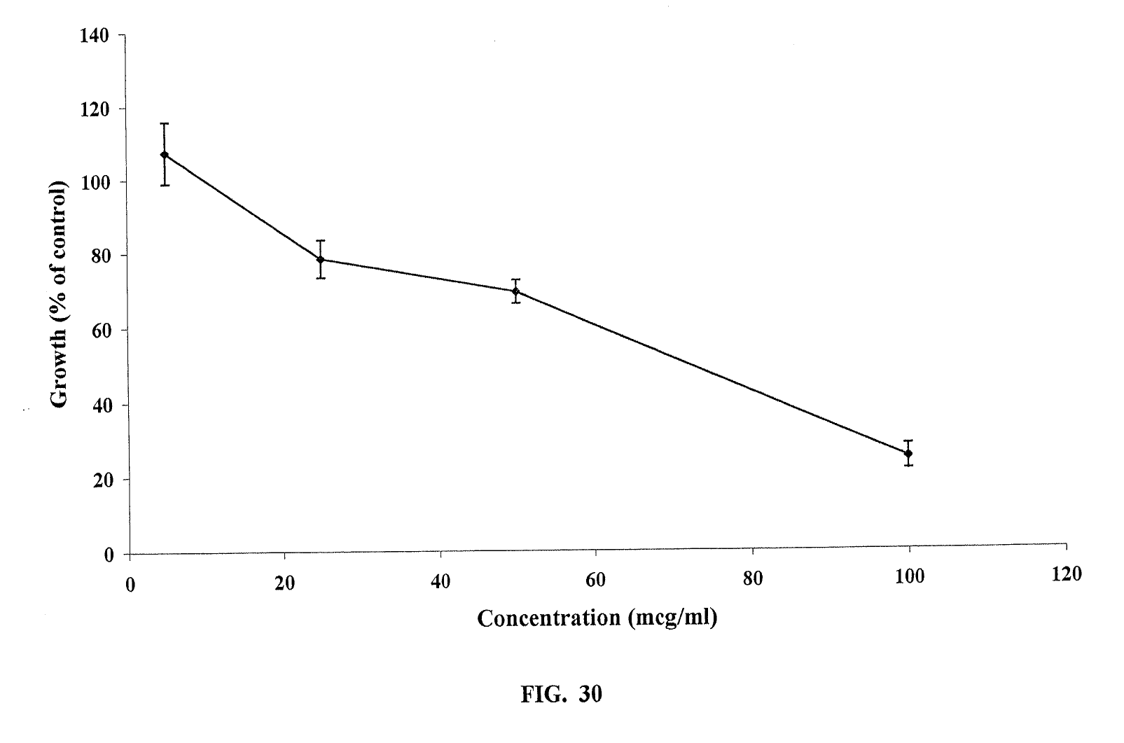

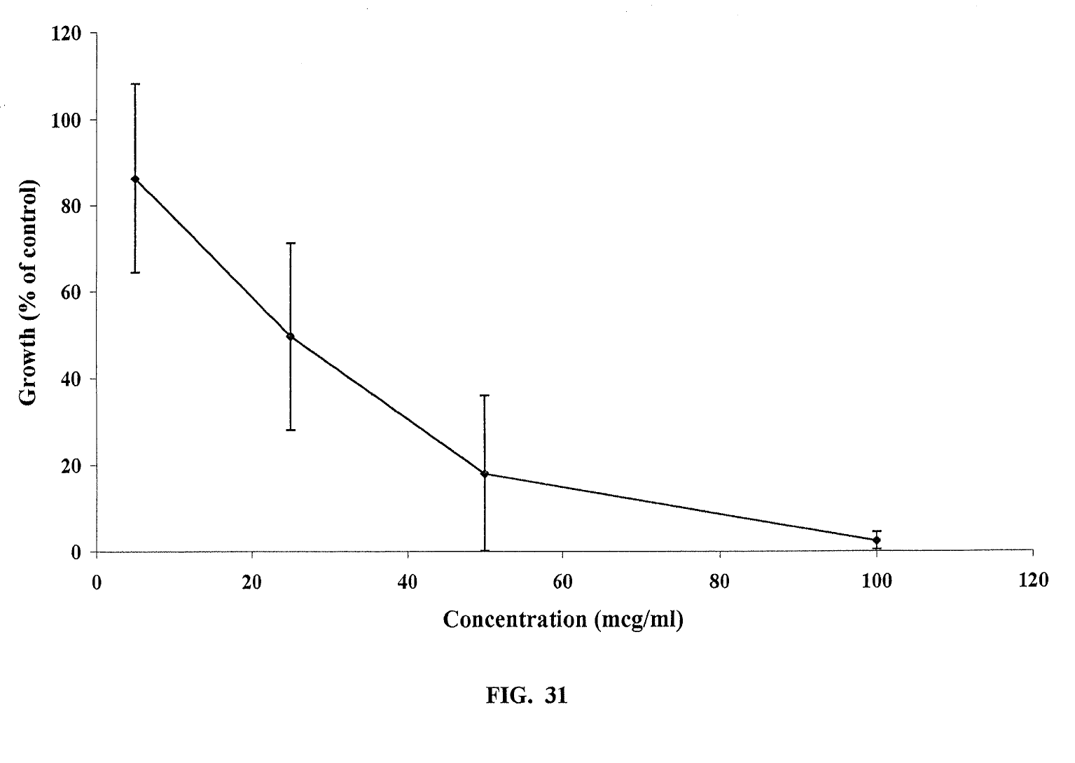

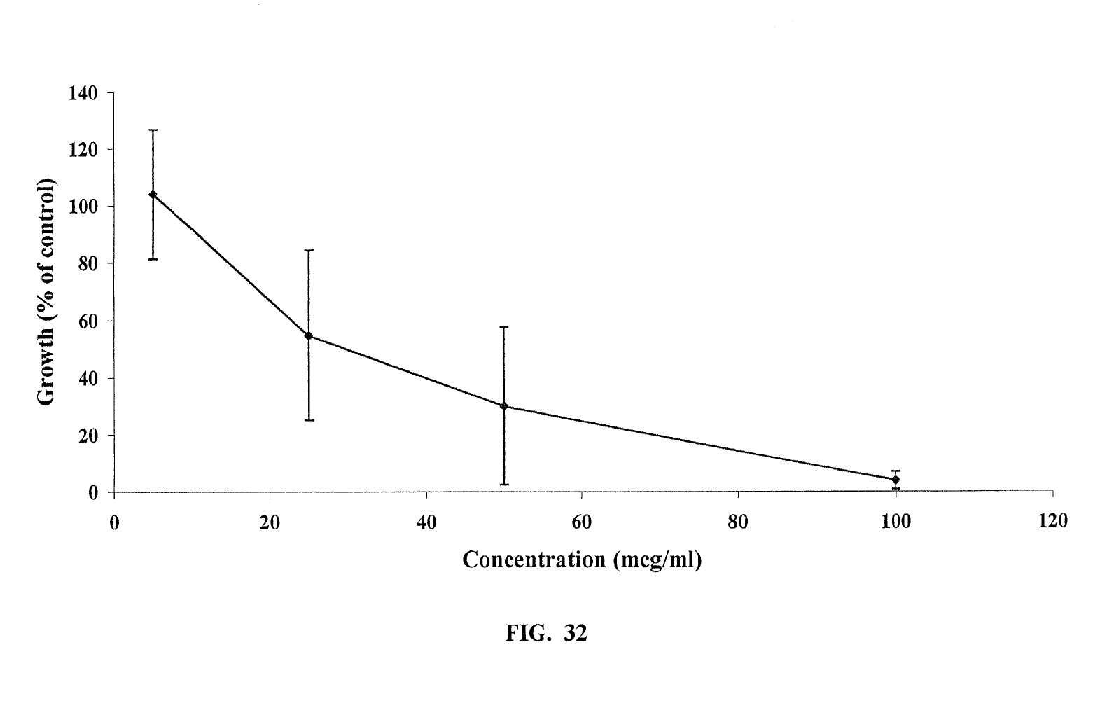

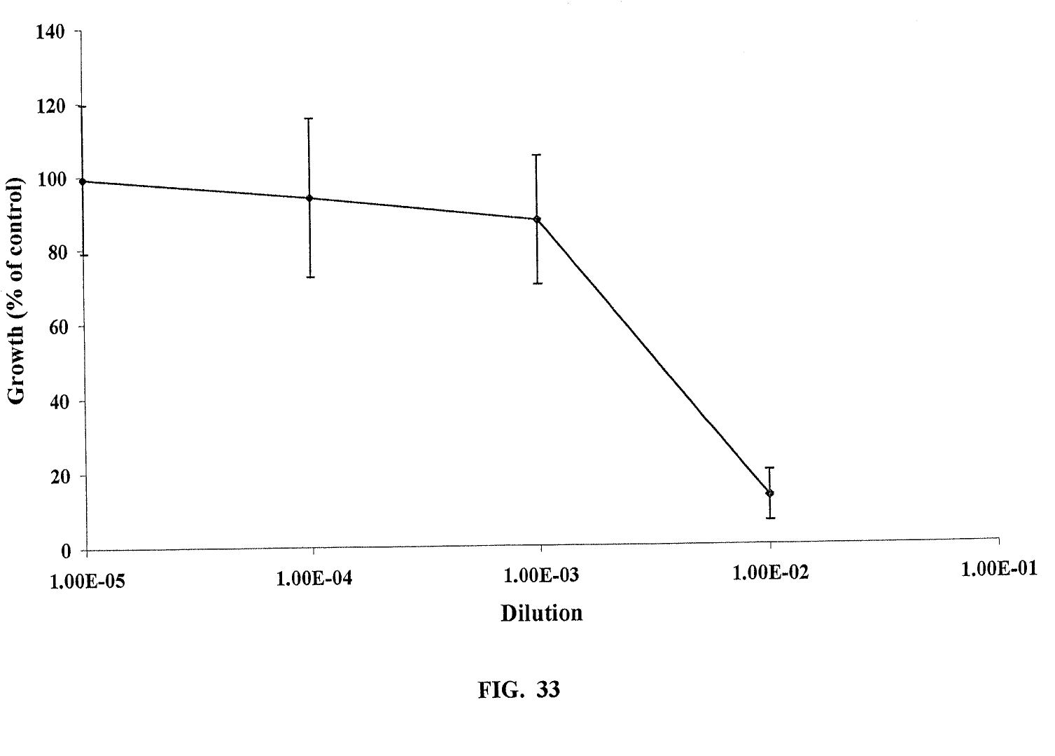

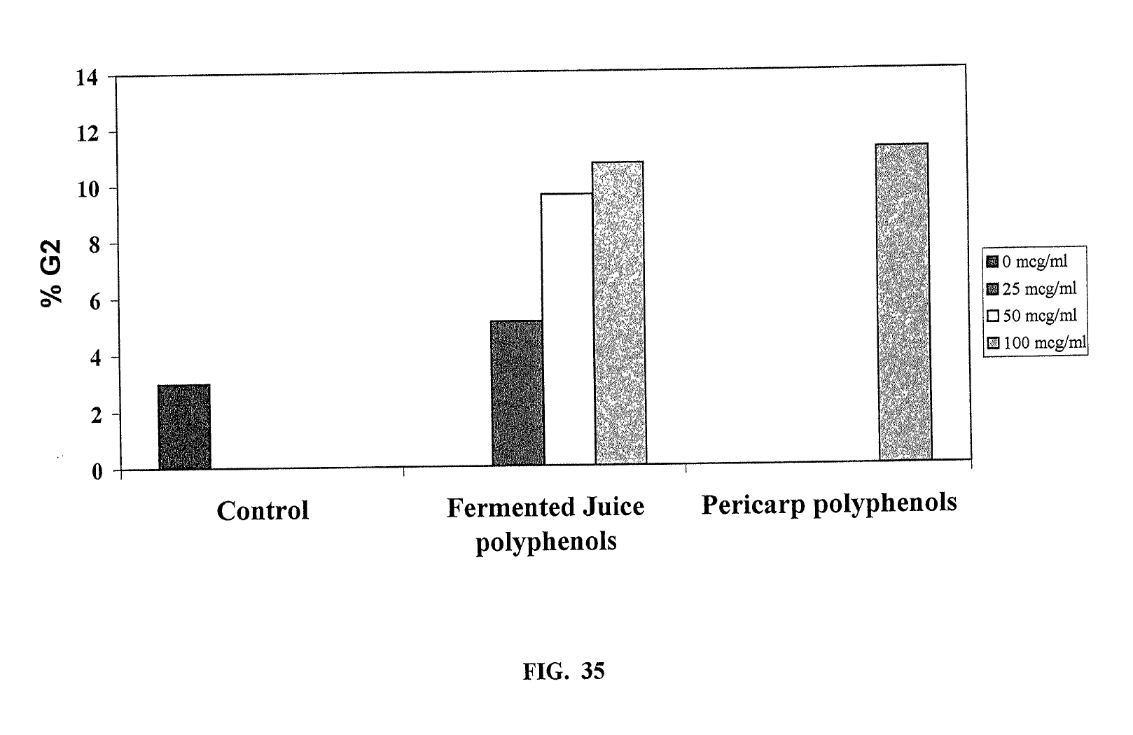

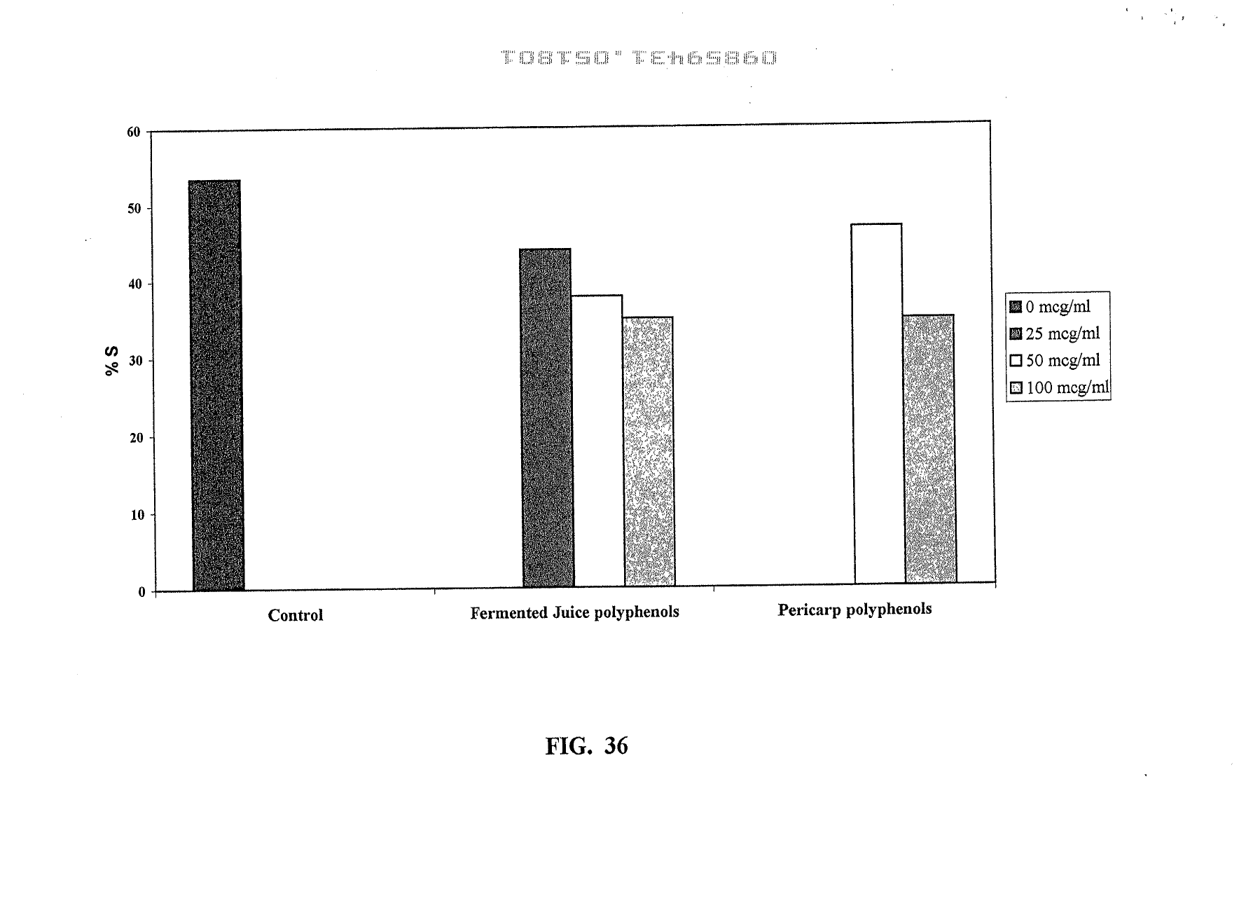

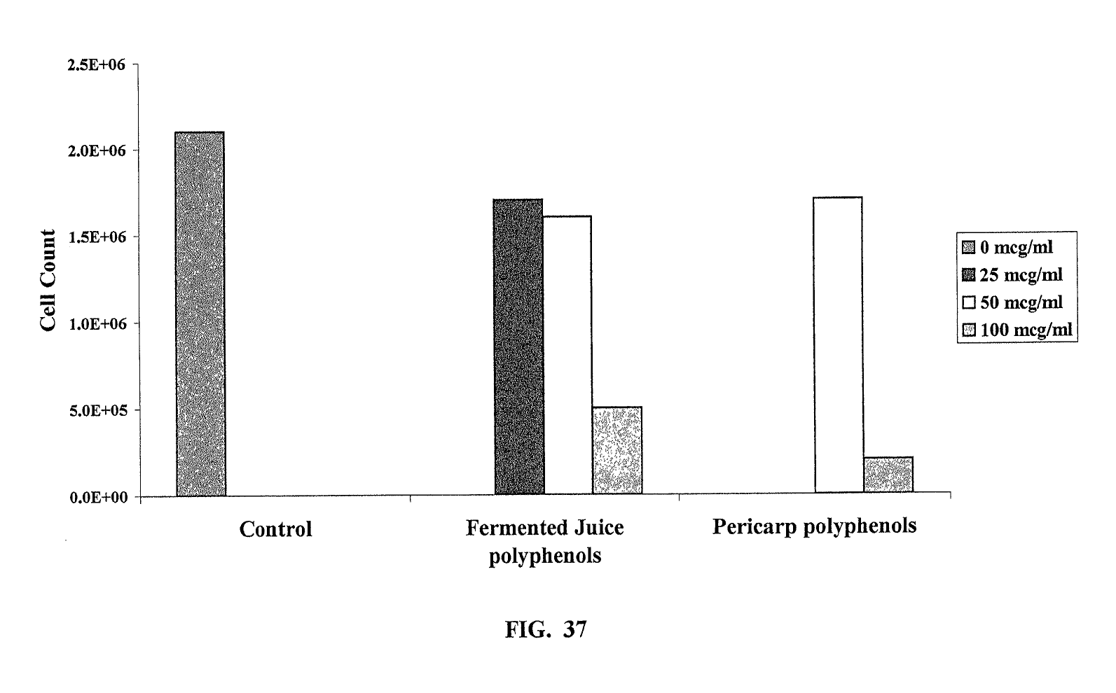

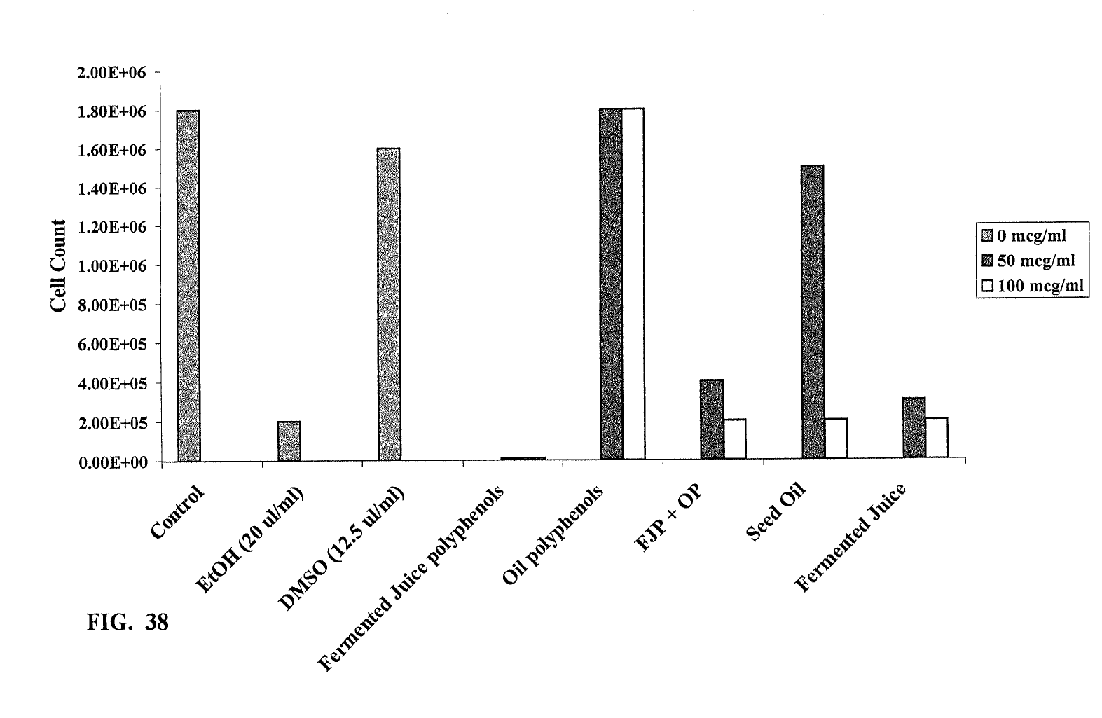

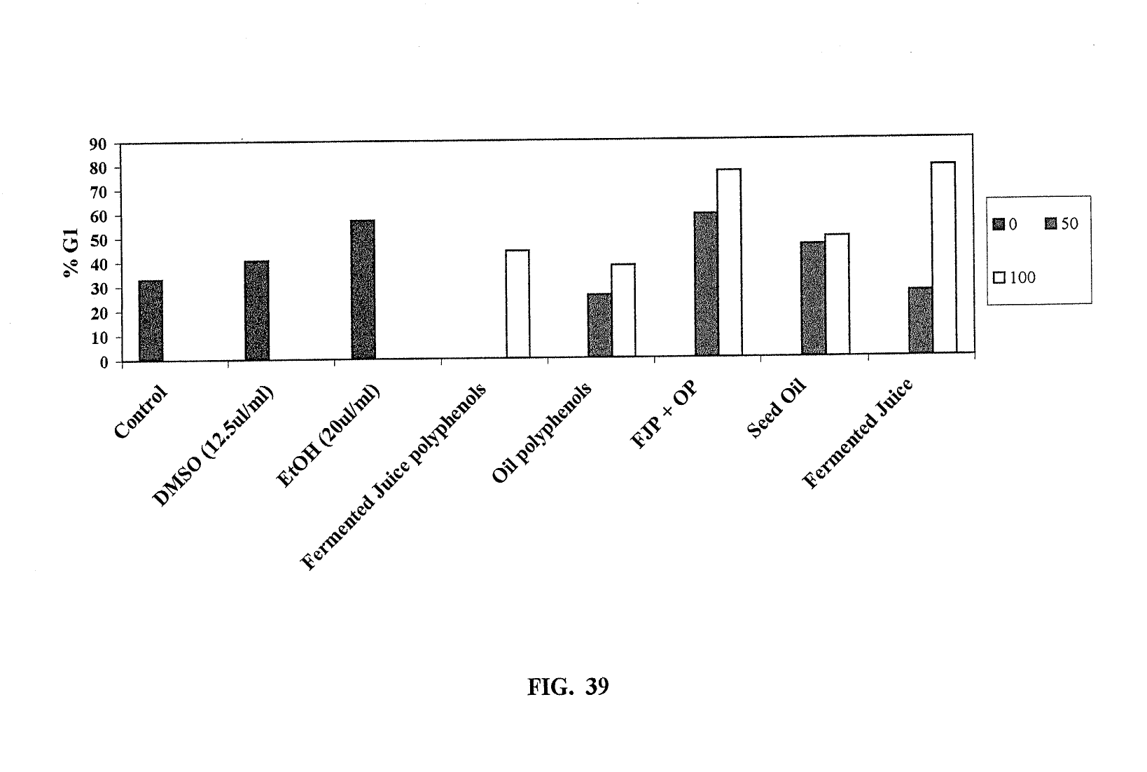

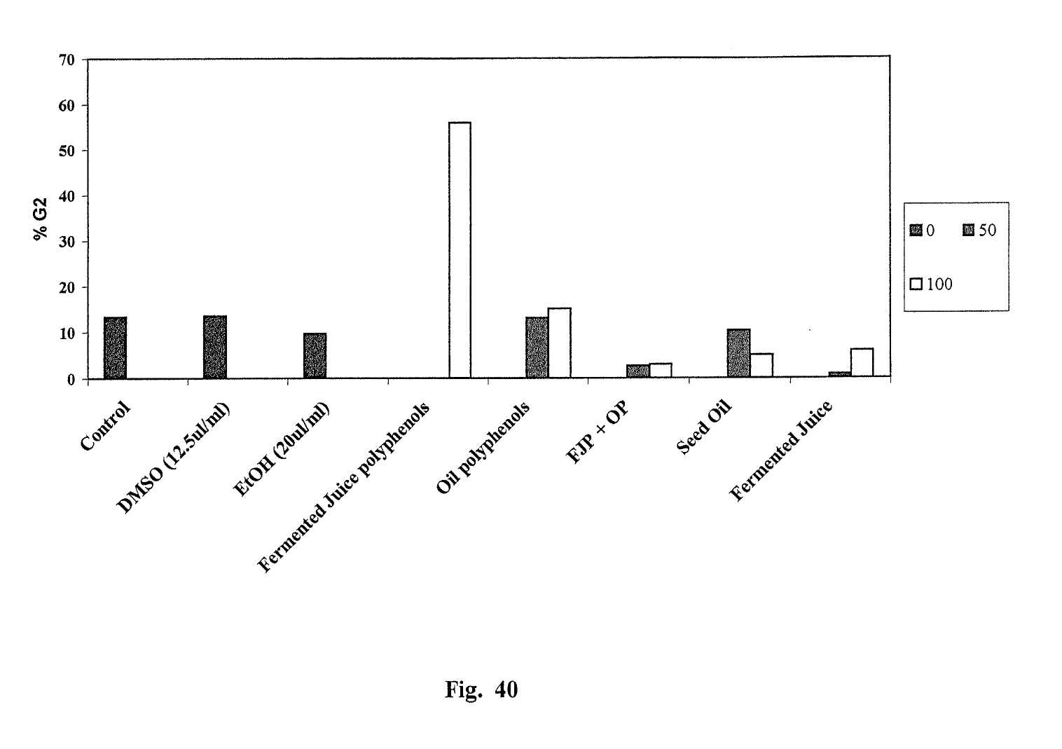

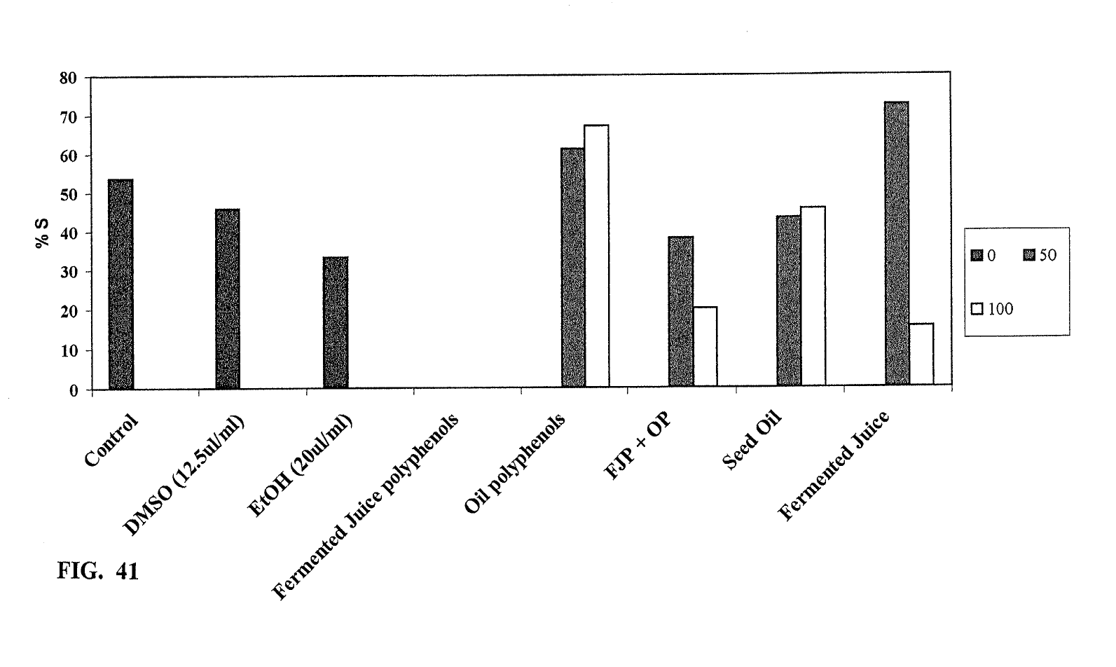

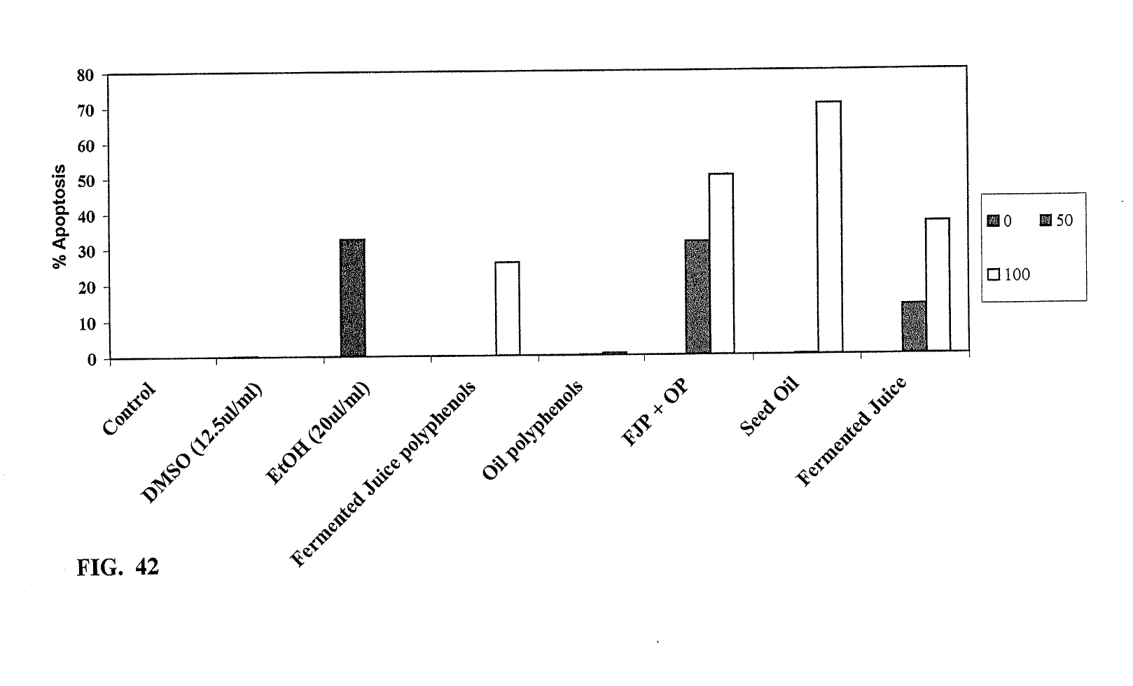

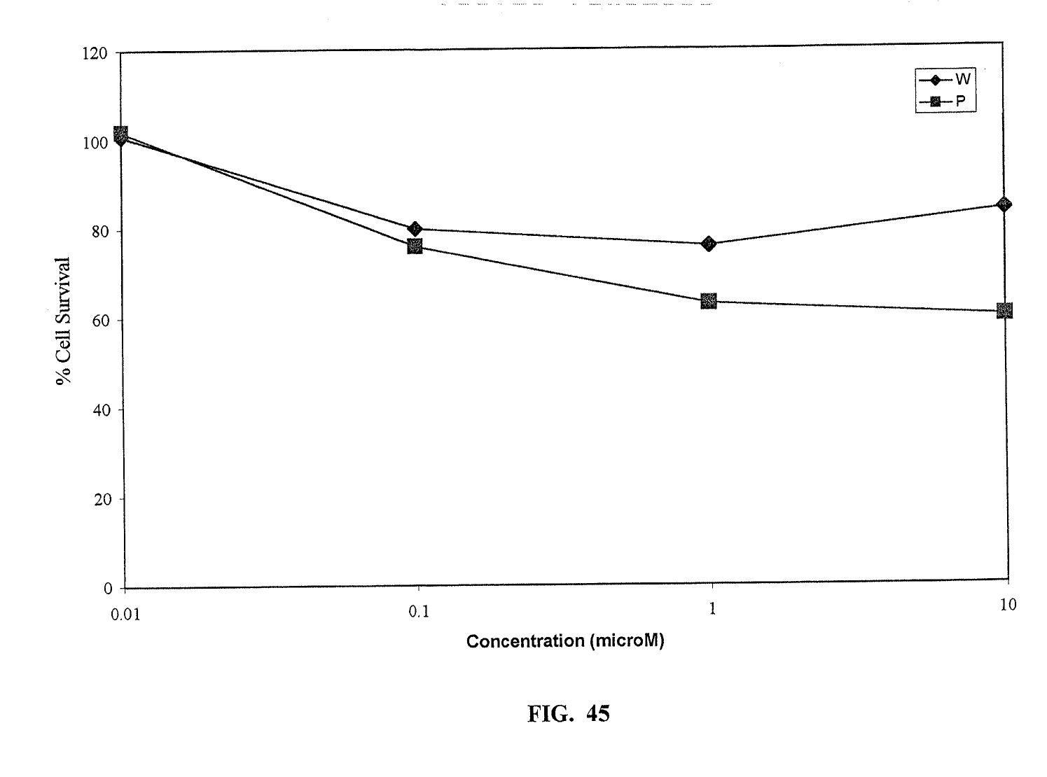

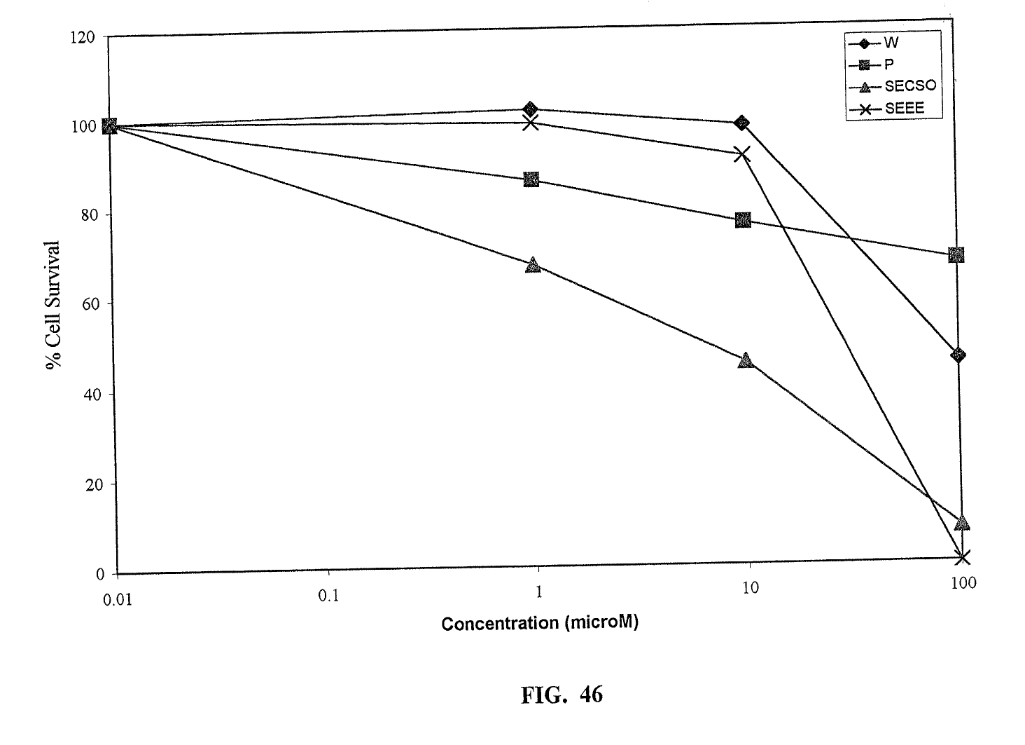

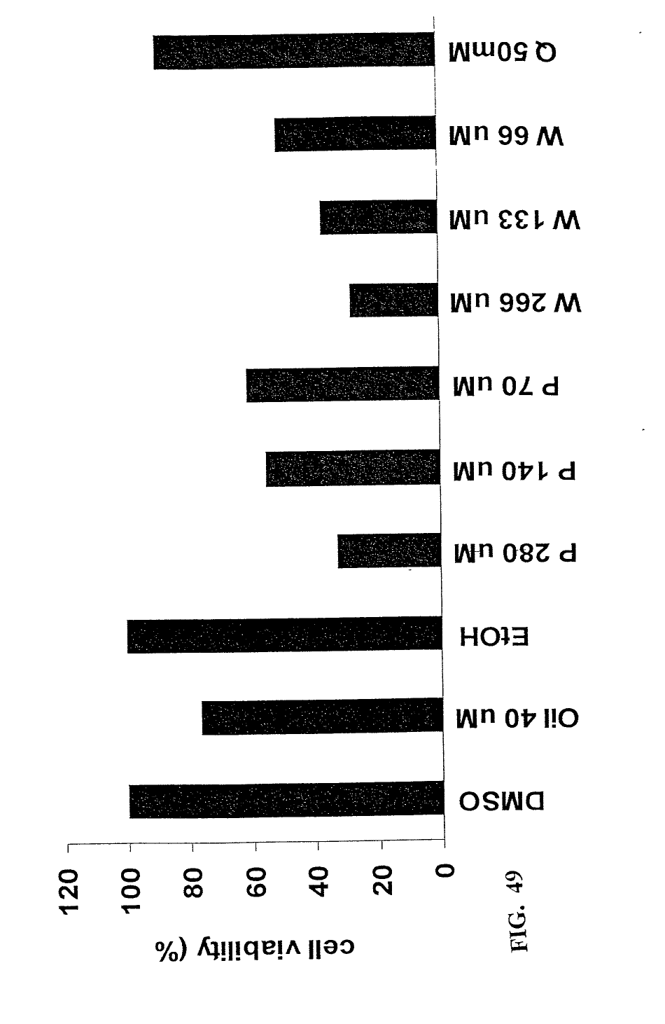

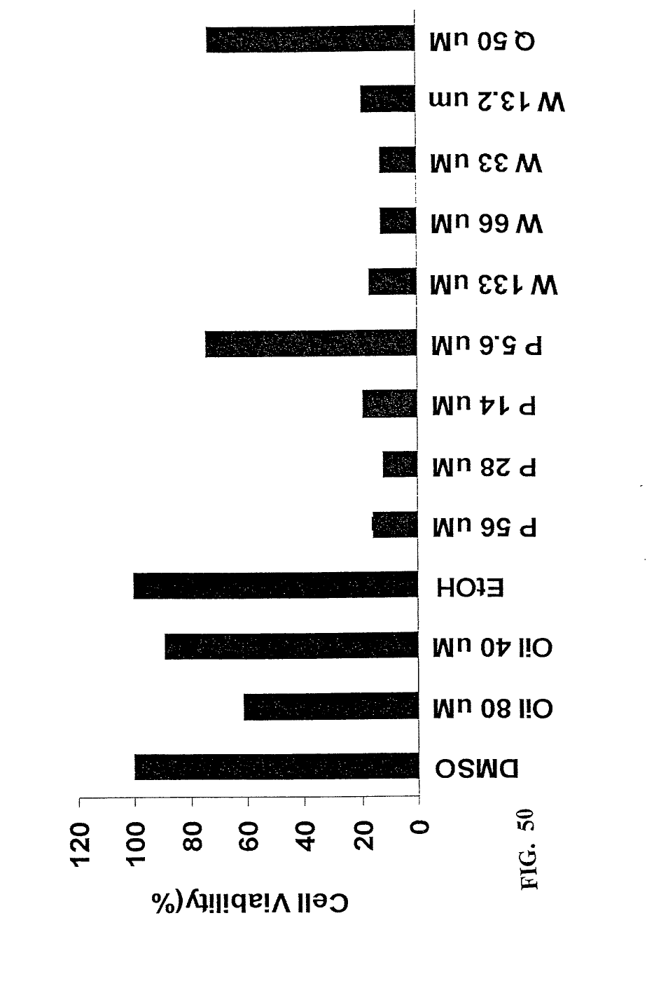

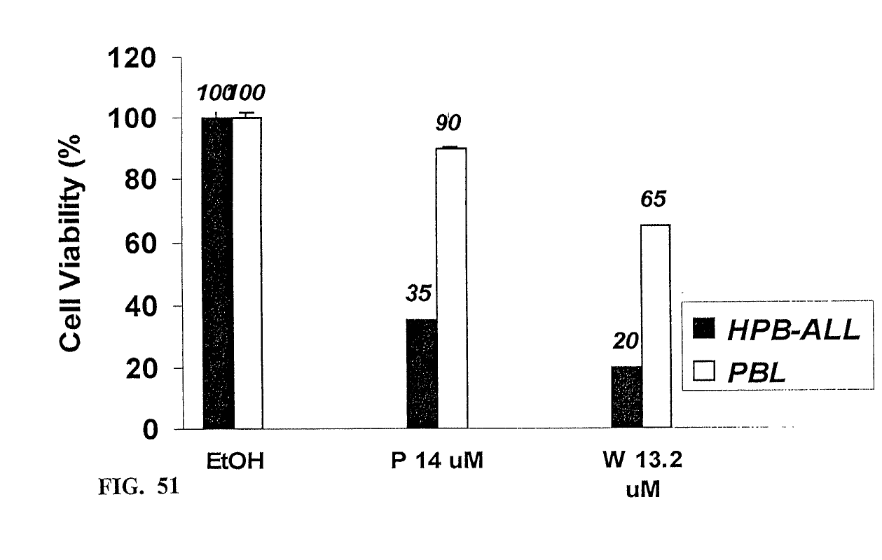

Based upon results presented in examples hereinbelow, the physiologic activity of mixtures and pharmaceutical compositions of the present invention is believed to result from inhibition of enzymes including, but not limited to, aromatase and 17-beta-hydroxysteroid dehdrogenase (HSD) type 1. However, the nature of the experimental work is empirical, and the possibility that additional metabolic pathways are involved is high. FIGS. 1-51 are explained in the context of examples 1-12 hereinbelow.

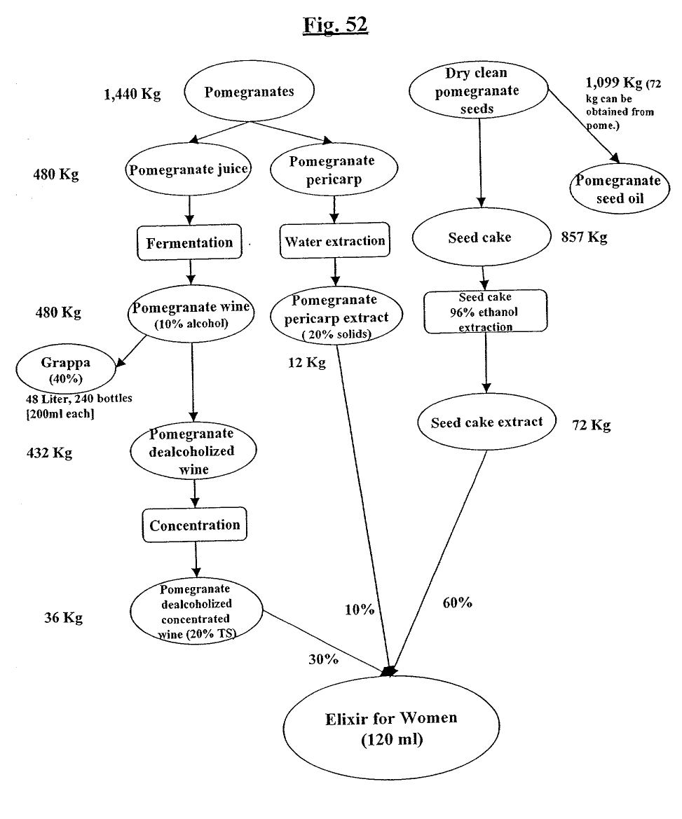

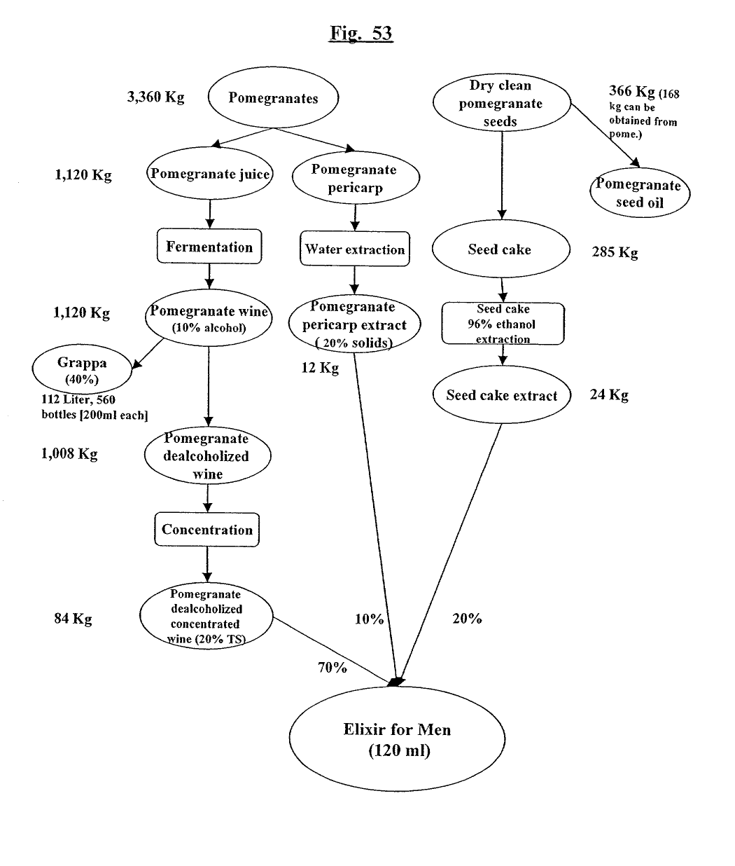

As disclosed in detail in example 13 hereinbelow (see also FIGS. 52 and 53) the invention is specifically embodied by a mixture or pharmaceutical composition which includes as active ingredients dealcoholized concentrated pomegranate wine, aqueous extract of pomegranate pericarp, and seed cake extract. According to specific preferred embodiments of the invention, the ingredients are present in a ratio of approximately 70% dealcoholized concentrated pomegranate wine, approximately 10% aqueous extract of pomegranate pericarp, and approximately 20% seed cake extract. According to specific preferred embodiments of the invention, the ingredients are present in a ratio of approximately 30% dealcoholized concentrated pomegranate wine, approximately 10% aqueous extract of pomegranate pericarp, and approximately 60% seed cake extract.

The present invention is further embodied by a novel selective estrogen receptor modulator (SERM) derived from pomegranate. The novel SERM may include, for example, a pomegranate seed product, a pomegranate juice product or a pomegranate peel product. The pomegranate seed product may be the result of a process such as, for example, cold pressing, expeller pressing, supercritical fluid extraction with carbon dioxide, or lyophilization. Therefore, the pomegranate seed product may include pomegranate seed oil, an unsaponified fraction thereof, an alcohol extract of pomegranate seed cake pomegranate seed cake or combinations thereof. The alcohol employed to produce the alcohol extract is usually methanol, ethanol, propanol, butanol or combinations thereof although pentanol and longer chain alcohols may also be employed. The pomegranate juice product may include, for example, pomegranate juice, fermented pomegranate juice, dried pomegranate juice, dried fermented pomegranate juice, partially fermented pomegranate juice, partially dried pomegranate juice, partially fermented partially dried pomegranate juice, reduced pomegranate juice, partially reduced pomegranate juice and lyophylysates and supercritical fluid extracts thereof.

The pomegranate peel product may include, for example, pomegranate peel residue present in pomegranate juice as a result of a juicing process, an aqueous extract of pomegranate peel, an alcohol extract of pomegranate peel, an extract performed with an organic solvent which is not an alcohol, and a supercritical fluid extract of pomegranate peel. The supercritical fluid extract may be conducted with, for example, CO₂, ethanol, methanol, water, or combinations thereof.

The present invention is also embodied by a method for selectively modulating an estrogen receptor. The method includes the step of providing a pomegranate seed product, a pomegranate juice product, a pomegranate peel product or combinations thereof and the step of allowing a SERM contained in the at least one item to selectively modulate an estrogen receptor.

Additional embodiments of the present invention include novel methods of producing 17 alpha estradiol, gamma tocopherol and campesterol by extracting oil from pomegranate seeds and purifying the 17 alpha estradiol, gamma tocopherol or campesterol therefrom. One ordinarily skilled in the art of biochemistry will be able to effect such extractions using commercially available reagents.

Further additional embodiments of the present invention include novel methods of producing a composition containing a physiologically active amount of 17 alpha estradiol, gamma tocopherol or campesterol. The method includes the step of extracting oil from pomegranate seeds, the oil contains the physiologically active amount of 17 alpha estradiol, gamma tocopherol or campesterol.

The present invention is further embodied by a novel method for use of pomegranate seed oil. The method includes the step of administering the pomegranate seed oil to a subject to exert an effect on a physiologic process. The subject may be either an animal or a human.

The physiologic process may be, for example aging, apoptosis, prostaglandin biosynthesis, estrogen activity, development of elasticity, development of tone, humectification, formation of age spots, crosslinking of collagen, oxidation of LDL or telomerase activity. The effect may include, for example, an antioxidant effect. The antioxidant effect may be at least partially produced by at least one item selected from the group consisting of 17 alpha estradiol, gamma tocopherol and campesterol. Possible effects may include, but are not limited to retarding aging, retarding apoptosis, inhibition of prostaglandin biosynthesis, agonism of estrogen activity, increasing elasticity, increasing tone, increasing humectification, dissipating age spots, retarding the process of collagen crosslinking, retarding LDL oxidation and stimulating telomerase activity. The step of administering may include either local or systemic administration. Local administration may include, for example, topical application to skin, intravaginal application, intrarectal application, intranasal application, intraocular application and inhalation. Systemic administration may include, for example, oral administration, injection, subcutaneous implantation, intravaginal application, intrarectal application, and inhalation. Preferably, the pomegranate seed oil is administered together with, for example, a pomegranate juice product, a pomegranate peel product or a combination thereof.

The present invention is further embodied by an improved method of retarding the process of atherosclerosis. The method includes the steps of: fermenting juice extracted from pomegranates; and administering the fermented juice to a patient. According to this embodiment of the invention, administration is preferably systemic as defined hereinabove, most preferably via an oral administration route. In some cases methods according to the present invention include the additional step of further administering a pomegranate seed product with fermented juice. The pomegranate seed product may include, for example, pomegranate seed oil or an unsaponified fraction thereof.

Various embodiments of the present invention may further constitute a composition for inhibiting cellular aging. Such a composition includes an effective amount of a pomegranate product as defined hereinabove.

Methods according to the present invention may include the additional step of combining two, or more preferably three or more pomegranate products (as defined hereinabove) prior to administration.

The step of providing a pomegranate juice product according to the present invention preferably includes at least one process including, but not limited to, extracting juice from a pomegranate, fermenting pomegranate juice, drying pomegranate juice, drying fermented pomegranate juice, partially fermenting pomegranate juice, partially drying pomegranate juice, partially fermenting partially dried pomegranate juice, reducing pomegranate juice, partially reducing pomegranate juice and lyophylysation of a product of any process belonging to the group. Provided juice may further contain material derived from a pomegranate peel product such as, for example, residual pomegranate peel present in the juice when extracted from the pomegranates. Alternately or additionally, pomegranate peel may be added to the juice product.

In cases where juice is fermented, the fermentation may serve to at least partially de-glycosylate flavonoids in the juice, thereby enhancing their bio-availability.

For purposes of this specification and the accompanying claims, the phrase “pomegranate product” includes a pomegranate seed product, a pomegranate juice product and a pomegranate peel product as well as products derived from pomegranate blossoms. Further included in this definition is honey produced by bees fed a pomegranate product.

Pomegranate products according to the present invention, especially pomegranate seed and derivatives thereof, are preferably prepared from Wonderful cultivar pomegranates. More preferably, these pomegranates are organically grown, still more preferably, they are grown at Kibbutz Sde Eliahu in Israel.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

General references to standard laboratory techniques are provided throughout this document. The procedures therein are believed to be well known in the art and are provided for the convenience of the reader. All the information contained therein is incorporated herein by reference.

Materials and Methods:

Reference is now made to the following materials and methods which are employed in the examples detailed hereinbelow.

The Yeast Estrogen Screen (YES) was performed according to the method described by Arnold et al⁷. The yeast (strain DY150) contains the yeast expression plasmid containing the human estrogen receptor (hER) and the estrogen-sensitive Lac-Z reporter plasmid. The special yeast used was supplied courtesy of Dr. John McLachlan, Tulane-Xavier Center for Bioenvironmental Research, New Orleans, La. 70112, USA. The yeast was grown overnight in the presence or absence of 17-beta estradiol in a concentration of 0.4 μM. One set of estradiol samples was also incubated with freeze dried pomegranate juice (1 mg/100 μl MeOH). Another set of samples was incubated with the pomegranate juice only. All samples were tested in triplicates. ⁷ Arnold, S. F., Robinson, M. K., Notides, A. C., Guillette, L. J. and McLachlan, J. A. A yeast estrogen screen for examining the relative exposure of cells to natural and xenoestrogens. Environmental Health Perspectives 104 (5): 544-548, 1996.

The aromatase assay was carried out by contract on coded samples of the putative inhibitors. The method depends of the release of tritiated water after aromatization of androstenedione, consistent with the method described by Rabe et al.⁸ The coded samples consisted of polyphenol fractions of the pomegranate seed oil, fermented juice and pericarp aqueous extract respectively. Aminoglutethamide, the known aromatase inhibitor, was used as a positive control at a concentration of 100 microM. The experimental pomegranate fractions were used at full strength, and at 50%, 10% and 5% dilutions. ⁸ Rabe, T., Rabe, D. and Runnebaum, B. New aromatase assay and its application for inhibitory studies of aminoglutethimide on microsomes of human term placenta. Journal of Steroid Biochemistry 17: 305-309, 1982.

Preparation of pomegranate polyphenol fractions was according to the method described by Schubert et al⁶. For the fermented juice and aqueous extract of the rinds, the original liquids were combined with two times their volume of ethyl acetate, shaken vigorously, and left for 8 hours. The ethyl acetate phase was then dried in the vacuum evaporator at 40 degrees centigrade, and polyphenols resuspended in methanol. ⁶ Schubert, S. Y., Lansky, E. P., and Neeman, I. Antioxidant and eicosanoid enzyme inhibition properties of pomegranate seed oil and fermented juice flavonoids. Journal of Ethnopharmacology 66 (1): 11-17, 1999.

Polyphenol extraction from cold pressed pomegranate seed oil was accomplished by moving a 10 gram aliquot with 50 ml hexane in a separation funnel and polyphenols extracted with three volumes of 60% methanol. The methanol phase was then moved to a second separation funnel and washed with 20 ml hexane. The methanol phase was then collected and dried with anhydrous Na₂SO₄ and again dried in a vacuum evaporator at 40 degrees. The resultant polyphenols were resuspended in methanol and extracted with three portions of chloroform, each half the volume of the methanol phase. The chloroform was removed and the methanol dried again in the vacuum evaporator at 40 degrees. The polyphenols were resuspended in water and extracted with petrol ether (60-80) until a clear organic phase was obtained. The water phase was saturated with NaCl and extracted with four portions of ethyl acetate (EA), each a third of the water phase volume. The EA fraction was collected and dried with anhydrous Na₂SO₄. The EA was dried in a vacuum evaporator and the polyphenols resuspended in methanol.

A non-saponifiable fraction (NSF) of pomegranate seed oil was prepared by combining a quantity of ethanol extracted pomegranate seed oil with KOH to produce a saponified mixture. This mixture was washed repeatedly with petroleum ether to obtain the NSF, which was subsequently dried with anhydrous sodium sulfate. According to published references, this NSF, when injected into mice and rabbits, exerted significant estrogenic activities as measured by ovarian weight and comification of vaginal epithelium.⁹ ⁹ Sharaf, A. and Nigm, S. A. R. The oestrogenic activity of pomegranate seed oil. Journal of Endocrinology 29: 91-92, 1964.

The MTT assay was performed as described in Ruben, R. L. and Neubauer, R. H. (1987) “Semiautomated calorimetric assay for in vitro screening of anticancer compounds”, Cancer Treat. Rep. 71(12):1141-9.

Nitro blue tetrazolium reducing activity was measured by the method described in Kawaii S., Tomono Y., Katase E., Ogawa K. and Yano M. (1999) “Effect of citrus flavonoids on HL-60 cell differentiation” Anticancer Res. 19(2A):1261-9.

Non-specific esterase activity was measured by the method of Rovera G., Santoli D. and Damsky C. (1979) “Human promyelocytic leukemia cells in culture differentiate into macrophage-like cells when treated with a phorbol diester” Proc. Natl. Acad. Sci. U S A 76(6):2779-83.

Specific esterase activity was measured by the method of Kawaii S., Tomono Y., Katase E., Ogawa K, and Yano M. (2000) “Effect of coumarins on HL-60 cell differentiation” Anticancer Res. 20(4):2505-12.

Phagocytic activity was measured by the method of Kawaii S., Tomono Y., Katase E., Ogawa K. and Yano M. (1999)“Isolation of furocoumarins from bergamot fruits as HL-60 differentiation-inducing compounds” J. Agric. FoodChem. 47(10):4073-8.

Cellular proliferation in human promyelocytic leukemia cells was measured by the method of Kawaii S., Tomono Y., Katase E., Ogawa K., Yano M., Takemura Y., Ju-ichi M., Ito C. and Furukawa H. (1999)“Acridones as inducers of HL-60 cell differentiation” Leuk Res. 23(3):263-9.

### Example 1

### Yeast Estrogen Screen of Pomegranate Derivatives

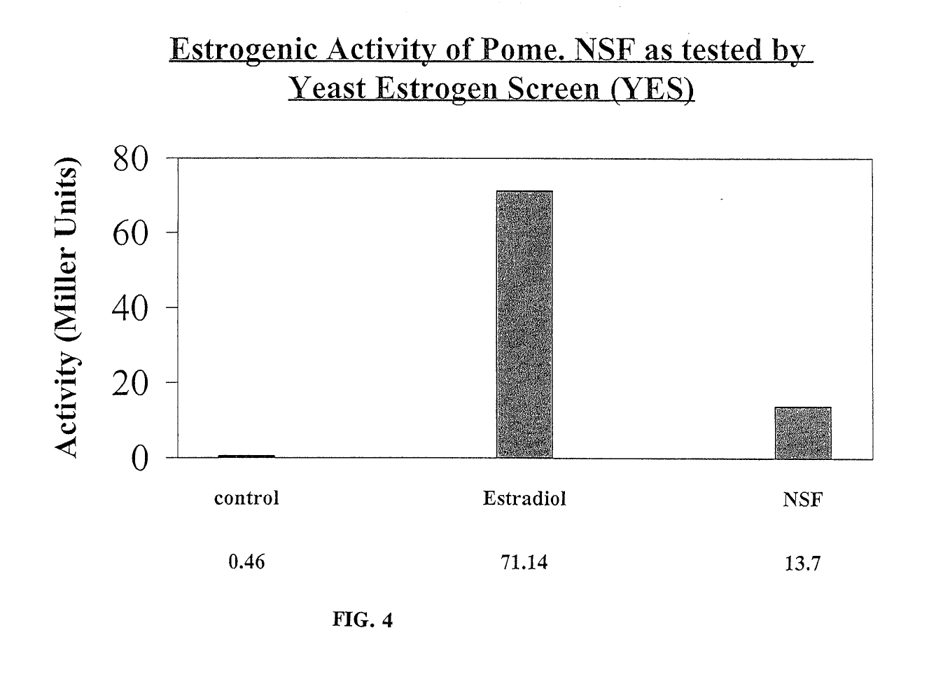

In order to test the anti-estrogenic activity of pomegranate juice (PJ) and estrogenic activity of NSF of pomegranate seed oil, a YES was performed. Results of the YES assays are presented in FIGS. 1 and 4. FIG. 1 shows inhibition of the estrogenic activity of the estrogenic standard 17-beta estradiol as a result of the addition of the pomegranate juice. 17-beta estradiol activity of 100 Miller Units was reduced to 50 Miller Units by the added pomegranate juice. Using a similar screen, (FIG. 4) NSF was shown to have an estrogenic activity about 20% that of a comparable concentration.

These data corroborate animal findings which suggest that pomegranate seed oil may have utility as an estrogen homologue with potential applications in treating climacteric women or male prostate cancer patients. Also corroborated is the earlier finding that pomegranate juice also may exert a weak estrogenic action parallel to its antiestrogenic potential.¹⁰ ¹⁰ Miksicek, R. J. Commonly occurring plant flavonoids have estrogenic activity. Molecular Pharmacology 44: 37-43, 1993.

### Example 2

### Assay of Aromatase Activity

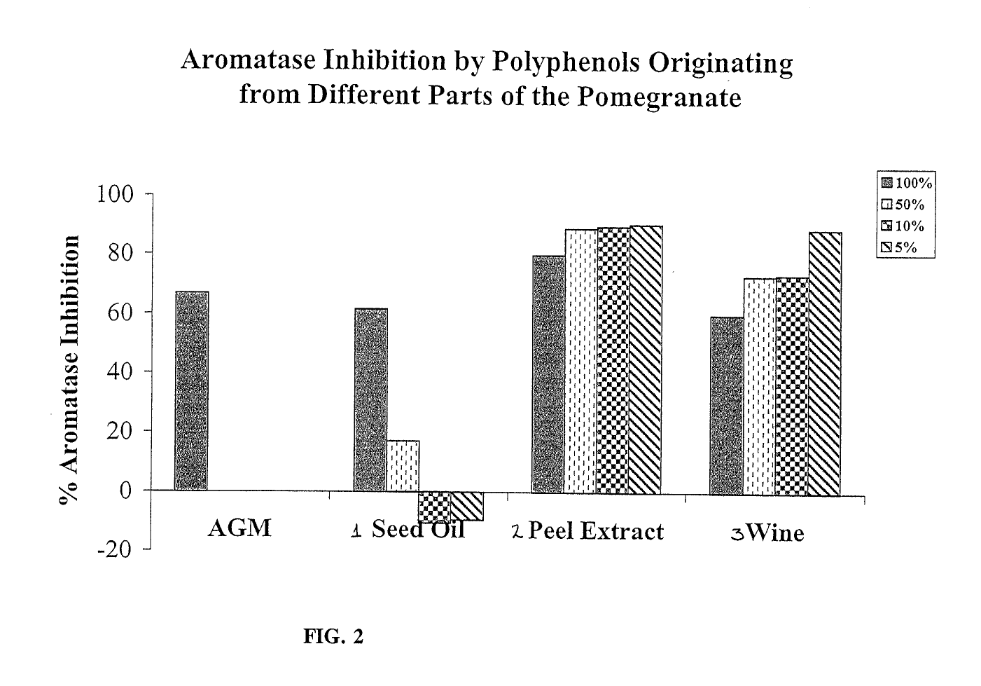

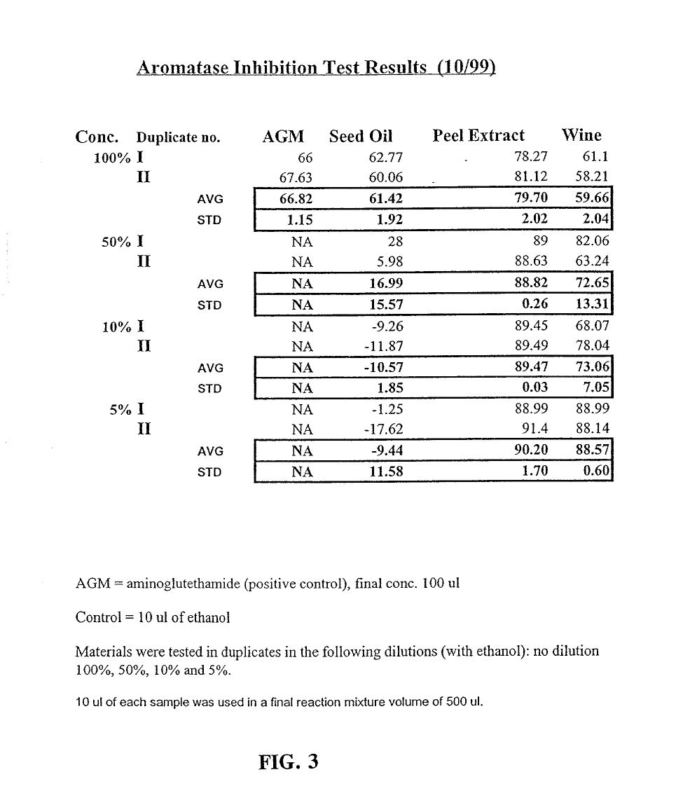

Results of the aromatase assay are summarized in FIGS. 2 and 3. The positive control aminoglutethamide (AGM) exhibited 66% inhibition of aromatase. The pomegranate seed oil extract (Sample One) showed a mean inhibition of 61% which was concentration dependent and rapidly dropped off at lower concentrations. The aqueous pericarp extract (Sample Two) showed a mean inhibition of 80% at full concentration, and at 50% actually increased to 89%. This level of inhibition was consistent all the way down to 5% dilution. The fermented juice (wine) extract (Sample Three) showed 60% inhibition at full strength, 70% inhibition at 50% dilution and 10%, and 88% inhibition at 5%. These results confirm and corroborate those of example 1 , suggesting potential medical utility for the assayed preparations of pomegranate. Aromatase inhibition and suppression of endogenous estrogenic activity are important factors in the control of breast cancer growth.¹¹ ¹¹ Dowsett, M., Macaulay, V., Gledhill, J., Ryde, C., Nicholls, J., Ashworth, A., McKinna, A. and Smith, I. E. Control of aromatase in breast cancer cells and its importance for tumor growth. Journal of Steroid Biochemistry and Molecular Biology 44 (4-6): 605-609, 1993.

### Example 3

### Antiproliferative Effects of Pomegranate Fermented Juice and Pericarp Extracts in Estrogen Receptor Positive (MCF-7) and Estrogen Receptor Negative (MDA-MB-231) Human Breast Cancer Cells in Culture

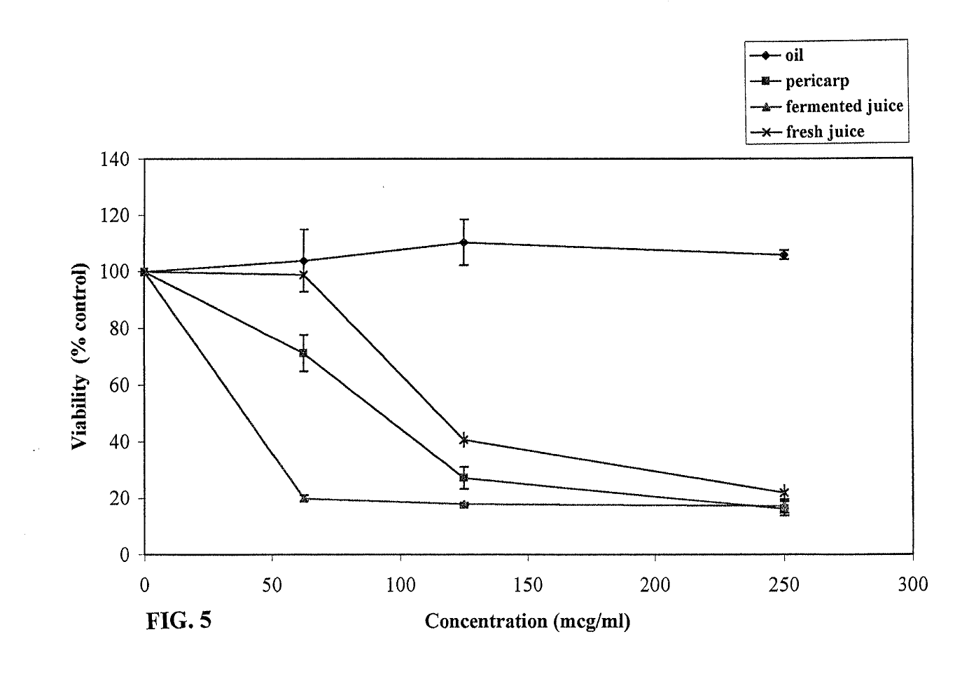

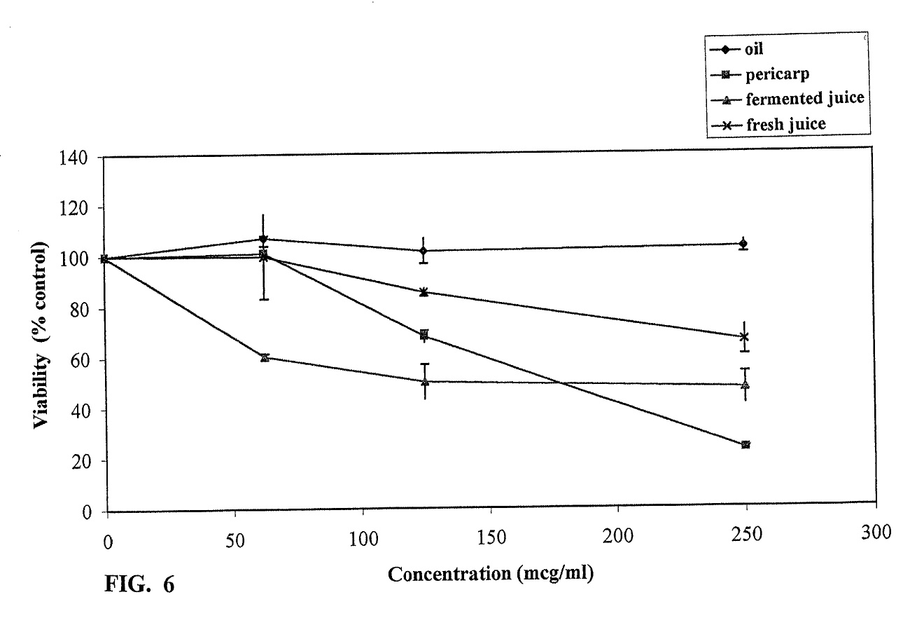

In order to test the effect of pomegranate fermented juice and pericarp extracts in estrogen receptor positive (MCF-7) and estrogen receptor negative (MDA-MB-231) human breast cancer cells in culture, polyphenol-rich fractions, consisting of flavonoids and tannins, were extracted from the seed oil, pericarp, unfermented and fermented juice of the pomegranate, Punica granatum. The different fractions were incubated in individual well plates with both estrogen receptor positive (MCF-7) and estrogen receptor negative (MDA-MB-231) human breast cancer cells for 48 hours. Cell viability was assessed with the MTT assay. Results are summarized in FIGS. 5 and 6. The fermented juice exerted the strongest overall anti-proliferative effect in both the MCF-7 and MDA-MB-231 lines. The second strongest in both lines was the aqueous pericarp extract. The unfermented juice also exerted significant anti-proliferative activity of the MCF-7 cells, but only mild anti-proliferative activity in the MDA-MB-231 cells. Overall, the effect in the MCF-7 lines for all pomegranate materials was more pronounced than that for the MDA-MB-231. Polyphenol fraction isolated from the pomegranate seed oil failed to have anti-proliferative effect in either of the assays at the concentrations employed. The IC50 for the fermented pomegranate juice polyphenol/flavonoid fraction was about 40 micrograms/ml for the MCF-7 cells and 120 micrograms/ml for the MDA-MB-231. These findings indicate that pomegranate fermented juice and pericarp decoction inhibit proliferation of both estrogen-positive and estrogen-negative breast cancer cells, apparently by two different mechanisms. Synergy between these mechanisms remains to be investigated.

### Example 4:

### Differentiation Promoting and Anti-proliferative Properties of Pomegranate Fermented Juice and Pericarp Extracts in Cancer and Normal Cell Lines

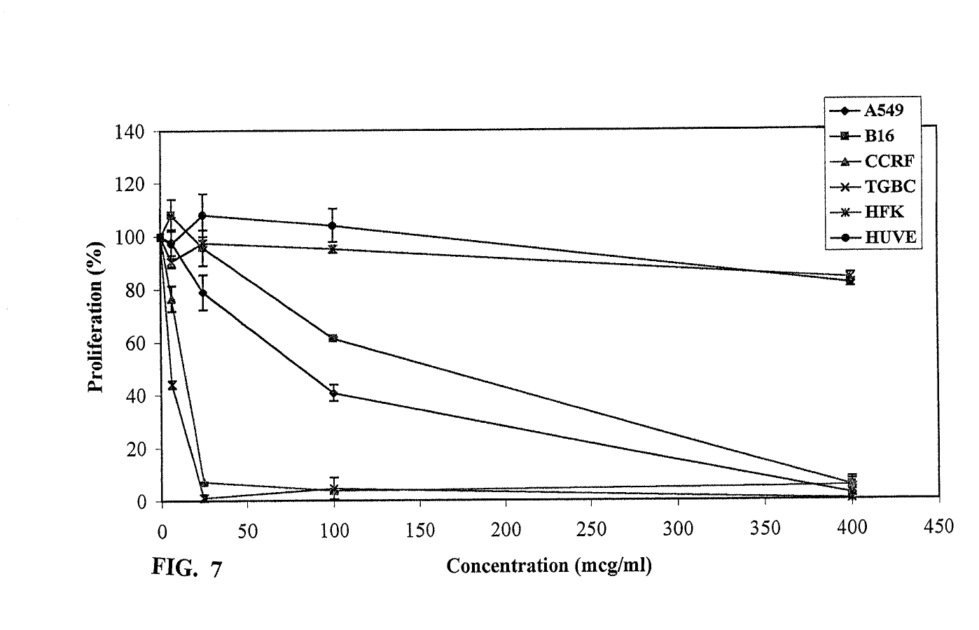

In order to assess the ability of pomegranate fermented juice and pericarp extracts to promote differentiation and prevent proliferation in cancer and normal cell lines, polyphenol-rich fractions were extracted from fresh and fermented pomegranate juice, expeller-pressed pomegranate seed oil and an aqueous decoction of pomegranate pericarps (rinds) utilizing chemical solvents. The four different fractions were then individually tested in proliferative assays in normal (human umbilical epithelium—HUVE, human foreskin keratinocyte—HFK) and cancer (human lung carcinoma—A-549, melanin pigment producing mouse melanoma—B16 melanoma—4A5, human T-cell leukemia—CCRF-HSB-2 and human gastric cancer lymph-node metastasized—TGBC11TKB) cell lines. Differentiation-inducing activity was assessed by nitro blue tetrazolium reducing activity, non-specific esterase activity, specific esterase activity, phagocytic activity and cellular proliferation in human promyelocytic leukemia cells (HL-60). Results are summarized in FIGS. 7-12. Very strong antiproliferative activity was observed for the fermented juice and aqueous pericarp extract in lymph node metastasized and human T-cell leukemia cell lines, and a milder activity for the non-fermented juice was observed in the same lines. Additional moderate antiproliferative activity was observed in mouse melanoma and human lung carcinoma cells with the pomegranate pericarp and fermented juice. The polyphenol fraction obtained from the oil was without effect in all cell lines, and the normal cells, i.e., the human foreskin keratinocytes and the human umbilical vein epithelium, were essentially unaffected by all pomegranate fractions though some mild antiproliferative activity was observed for the pericarp and fermented juice at very high concentrations. Differentiation-inducing activity was observed consistently for all parameters measured: fermented juice>/=pericarp>>unfermented juice>>>oil. These results indicate that the pomegranate fermented juice and pericarp fractions can inhibit cancer cells by promoting differentiation, differentiation being inversely correlated with cancer virulence. Inhibition of proliferation was also demonstrated in a number of key human and one murine cell lines, and an absence of proliferation inhibition noted in two key normal human cell lines. Thus from this data it appears that the pomegranate pericarp fraction specifically and significantly inhibit cancer cell growth and that the metastatic line was most strongly affected. Similarly, the most powerful antiproliferative effects are observed in the metastatic gastric lymph node and the T cell leukemia. The normal cell lines appear to be minimally affected with respect to proliferation. The effect of juice on proliferation is clearly milder than for the fermented juice. Again, the most sensitive lines are the metastatic gastric and the leukemia, while the normal cell lines are not affected.

### Example 5

### Inhibition of Invasion and Proliferation of Human MCF-7 Breast Cancer Cells and Inhibition of Proliferation of LNCapFGC Human Prostate Cancer Cells by Pomegranate Seed Oil in vitro

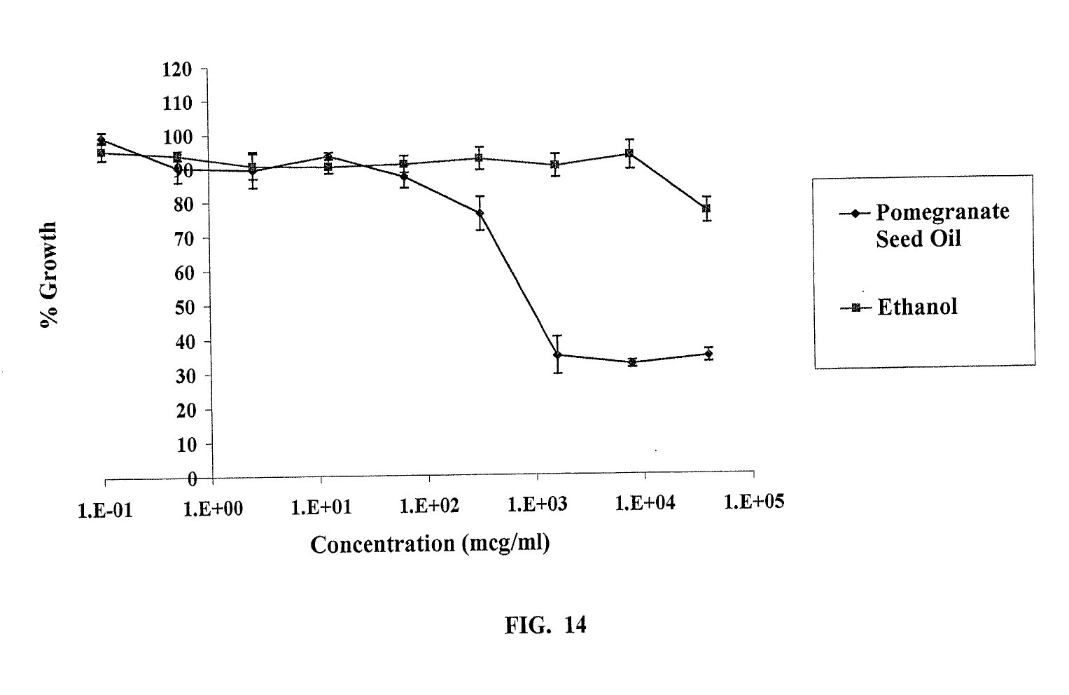

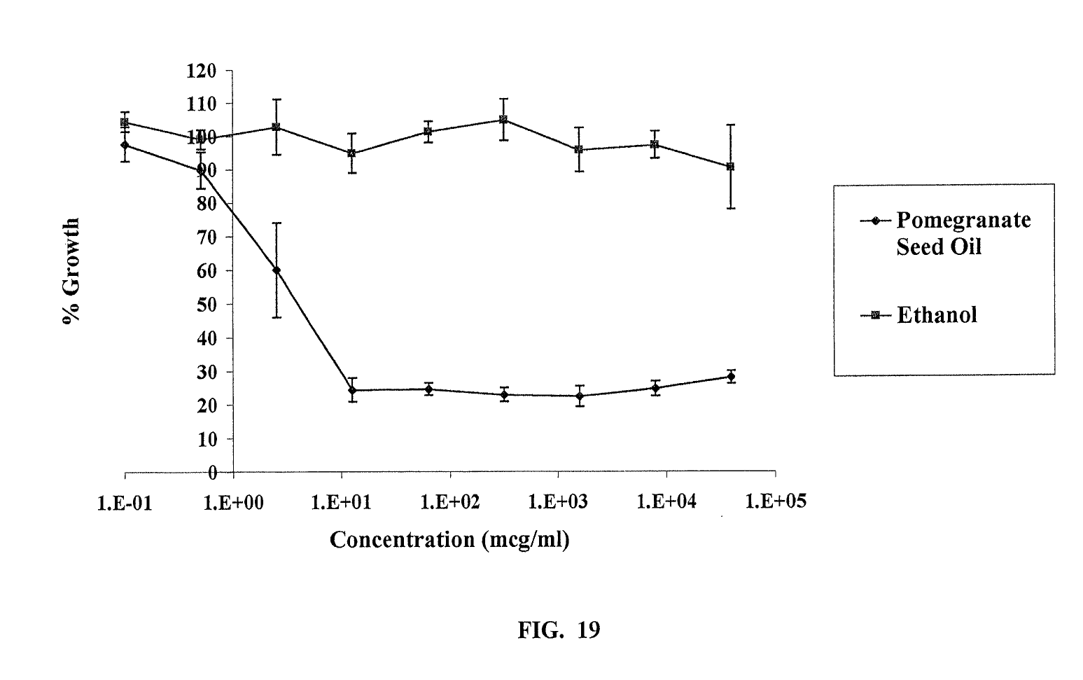

Pure expeller-pressed pomegranate seed oil (PSO) dissolved in ethanol was added to human MCF-7 human breast cancer cells, pre-labelled with a non-toxic fluorescent dye (1,1′-dioctadecyl-3,3,3′,3′-tetramethylindocarbocyanine) to a tissue culture of normal human umbilical vein endothelial cells (HUVEC). The degree of endothelial cell/tumor cell adhesions, a pre-condition for invasion and metastasis, was quantified with a fluorescent plate reader. The results (FIG. 13) demonstrated a significant inhibition of the experimental invasion by the PSO at dosages from 5 micrograms/ml of PSO in the cell medium. The effect increased linearly until about 10 micrograms/ml, gradually increasing until 200 micrograms/ml and then leveling off. An antiproliferative effect of the PSO employing a standard MTT assay (FIGS. 14-18) was noted in the same MCF-7 cell line from about 50 micrograms/ml, increasing linearly with dose and leveling off at 10,000 micrograms/ml, and in human LNCapFGC from about 1 micrograms/ml leveling off by 10 micrograms/ml (FIG. 19). These results demonstrate inhibition of breast cancer cell invasion by pure pomegranate seed oil at very low dosage. A tenfold greater concentration inhibits proliferation of the same cells. Inhibition of proliferation in prostate cancer cells was demonstrated at a dose approximately equivalent to that required for inhibition of invasion in breast cancer cells. In other words, prostate cancer cells were considerably more sensitive to the effects of the oil than the breast cancer cells.

### Example 6

### Inhibition of Estrogen Synthetase (Aromatase) by Flavonoids Derived From Selected Pomegranate Fractions

In order to elcucidate the possible molecular mechanism of pomegranate fractions on hormone dependent cancer cells, polyphenol-rich fractions were extracted from the seed oil, fermented juice and pericarp of Punica granatum using chemical solvents. Each fraction was individually tested by administering 10 microliters into the testing well of a human placenta system for aromatase inhibition using 10 microliters of 100 microMolar aminoglutethamide as a positive control. Samples were tested at 100%, 50%, 10%, 5%, 1% and 0.1% dilutions.

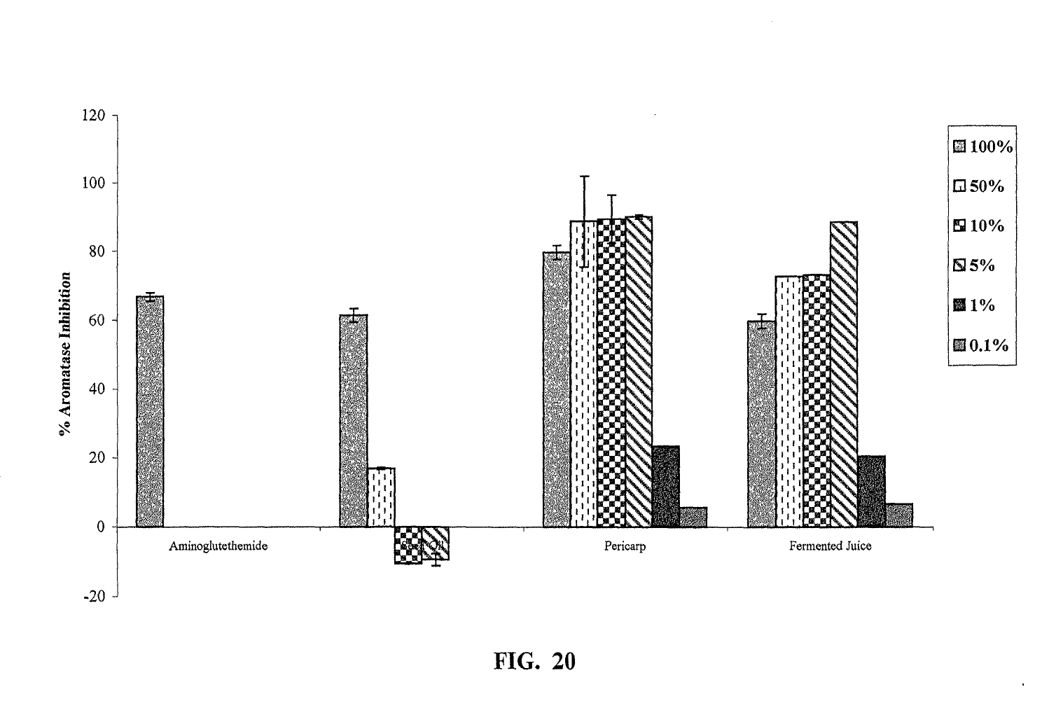

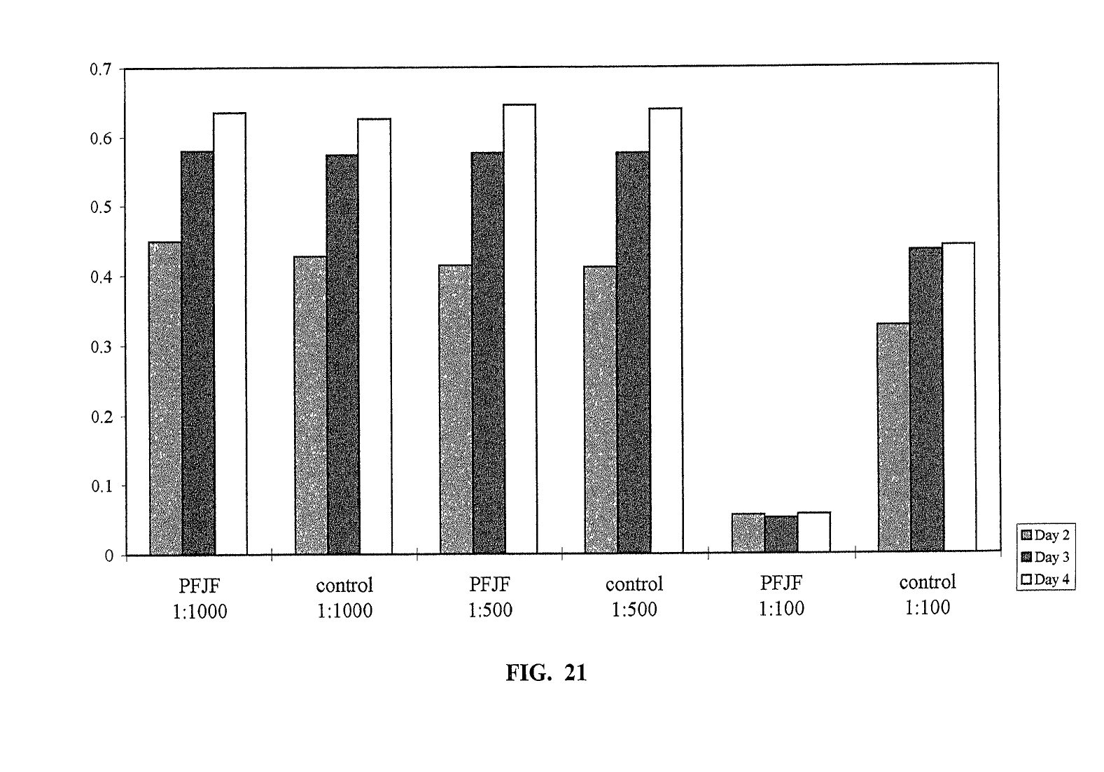

Inhibition was recorded as the percentage of inhibition achieved relative to that of the positive control, aminoglutethamide at 100 microMolar. Inhibition was strong in all fractions and was according to: pericarp>fermented juice>>oil. Inhibition was not attenuated even at 5% dilution with the pericarp and fermented juice fractions, but the oil showed a 50% attenuation by the 50% dilution, and no activity at 10% dilution and lower. Inhibition was still observed, though attenuated, at 1% and 0.1% dilution for the pericarp decoction and fermented juice polyphenol fractions. Results are summarized in FIG. 20. The P-450 enzyme, aromatase (estrogen synthetase), is responsible for catalyzing the biosynthesis of the steroidal estrogens estrone and 17-beta-estradiol from the androgens androestenedione and testosterone respectively in vivo. As this is a major biosynthetic pathway for the production of these strong estrogens, inhibition of aromatase is a popular and proven pharmacological method of retarding the development of estrogen-dependent breast cancers. These results suggest utility of pomegranate fermented juice and pericarp fractions in the treatment or prevention of breast cancer.

### Example 7

### Antiproliferative Activity of Pomegranate Fermented Juice and Seed Oil Flavonoids in Human Prostate Cancer (LNCaP, PC-3, DU-145) and Human Stromal Benign Prostatic Hypertrophy (hPCPs) Cells in vitro

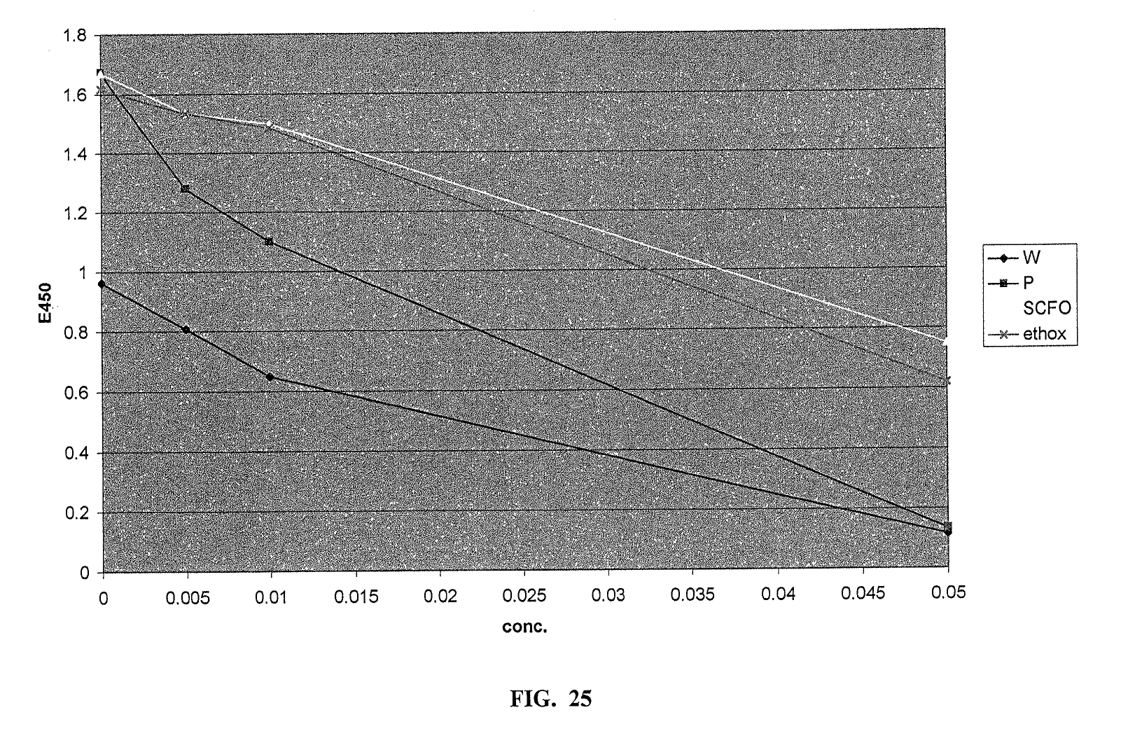

Human epithelial prostate cancer cells (LNCaP) and human stromal benign prostatic hypertrophy (BPH) cells (hPCPs) were seeded into 96 well plates and grown in the presence of varying dilutions of polyphenol fractions extracted from pomegranate seed oil and pomegranate fermented juice, and also in the presence of pure pomegranate fermented juice and pure pomegranate seed oil. On days 2, 3 and 4 cells were fixed, stained and the absorption measured. For each extract dilution and control, 8 samples were measured and mean value and standard deviation calculated. Results showed a strong inhibition of proliferation in both the prostate cancer and BPH cells by the pomegranate fermented juice flavonoids, and a milder inhibition by the full fermented pomegranate juice. Results are summarized in FIGS. 21-24. In all instances, the effect was more pronounced in the BPH cells (hPCPs) but was also strong in the cancer cells (LNCaP). Additional studies (Campbell, Geldof) focussed on the action of the aforementioned pomegranate compounds in more aggressive, more poorly differentiated, androgen-independent prostate cancer lines (i.e., PC-3 and DU-145). Results are summarized in FIGS. 25-33. In these lines as well, a significant inhibition of proliferation was noted with the fermented juice and pericarp extracts (W, P) but not with pomegranate seed oil. These results demonstrate an antimitogenic (antiproliferative) activity of pomegranate fermented juice and pomegranate fermented juice polyphenol fraction both in human prostate cancer cells and in human stromal BPH cells. This suggests a potential utility for pomegranate fermented juice fractions in the prevention and possibly also the treatment both of human prostate cancer and benign prostatic hypertrophy. The observance of the anti-proliferative effect even in the most poorly differentiated cells (DU-145) illustrates the potency and significance of this effect. Further studies are necessary to elucidate the mechanism of action, which clearly extends beyond suppression of the hormonal influence, as evidenced by results with PC-3 and DU-145.

### Example 8

### Localization of Antiproliferative Effects of Pomegranate Fractions to Specific Stages of the Cell Cycle

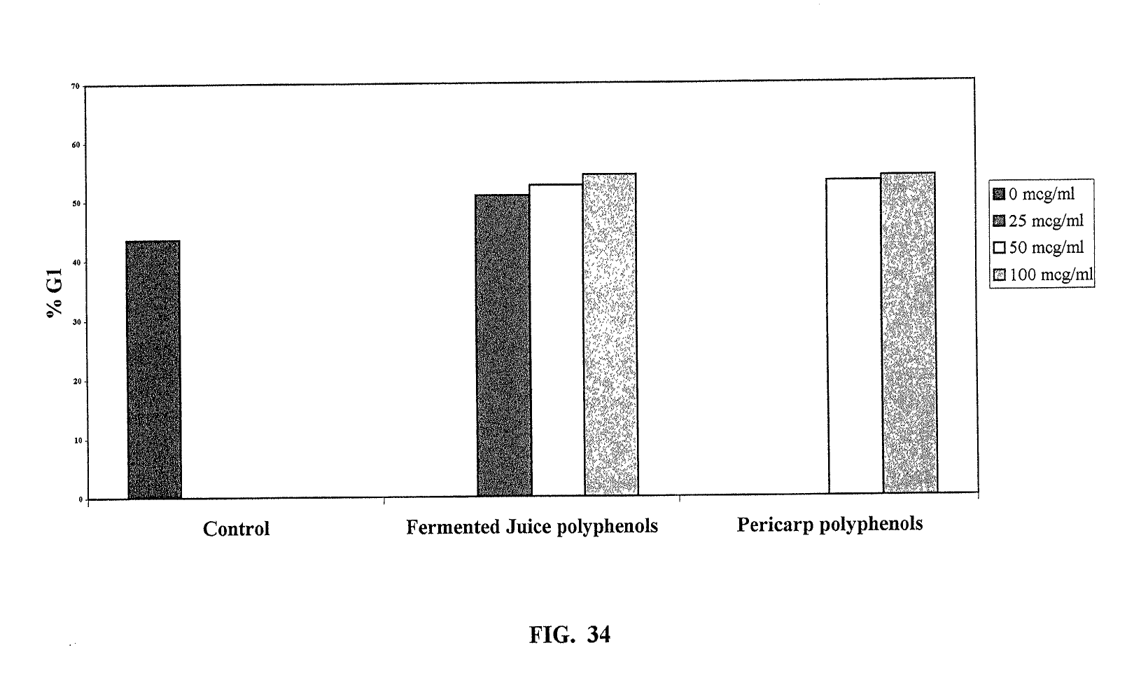

Ethyl acetate extracted polyphenol fractions of fermented pomegranate juice and an aqueous extract of pomegranate pericarps were assessed for their ability to suppress growth and to interrupt specific stages of the cell cycle in murine B16 (F 10) melanocytic melanoma and human HL-60 promyelocytic leukemia cells. Cells were grown in monolayer culture (35×10 mm flasks) in 3 mL of RPMI 1640 medium supplemented with 10% fetal bovine serum and 80 mg/L of gentamycin. Cultures, seeded with 3.3×10⁷ cells/L, were incubated for 24 h at 37 C in a humidified atmosphere of 5% CO₂. The media were decanted and replaced with fresh media containing the pomegranate fractions, and incubations were continued for additional time commensurate with the stage of the cell cycle being assessed. The medium and detached cells were decanted from cells grown in monolayer culture, and then incubated with trypsin-EDTA at 37 C for 2 min. Trypsin was inactivated by suspending the cells in medium containing 10% of FBS. The trypsinized cells were pelleted at 250× and resuspended in HBBS. Viable cells, [cells that excluded 0.4% of trypan blue], were counted with a hemocytometer. Pomegranate fermented juice polyphenols (W) were tested at 25, 50 and 100 micrograms solid material per ml of medium, while polyphenols derived from the pericarps (P) were tested at 50 and 100 micrograms per ml only. Results are summarized in FIGS. 34-42. Both W and P clearly caused inhibition of growth at GI while treatment with W also showed a steady lengthening of G2, suggesting a possible G2/M arrest. This occurred in both cell lines studied. Inhibition of the S phase (DNA synthesis) was pronounced for the W fraction. Investigation of the effects on apoptosis showed a dramatic apoptosis stimulating effect from the pomegranate seed oil, and considerably less even from the highest doses of the fermented juice polyphenols. These results suggest that the mechanisms underlying the antiproliferative effects of pomegranate fractions observed in cancer cells include influence on the normal cell cycle. Of special interest is that the seed oil and the aqueous fractions derived from the fermented juice and pericarp extracts seem to exert their antiproliferative effects in different ways suggesting that mixtures of these two fractions might be expected to exhibit synergy in vivo. The strongest degree of inhibition was observed with the polyphenol fraction of the fermented juice, and this effect was most dramatic at the G1, G2 and S stages. This material appears to inhibit cancer cell DNA synthesis (S). In general, the effect of the pericarp fraction of polyphenols (P) was similar to that of the fermented juice polyphenol fraction (W), though the effect of W is greater. For example, inhibition of glycosylation of hemoglobin was observed with W but not P, implying that W has an additional antioxidant activity. The effects at G1 and G2 are significant with both W and P, with the oil showing a much milder effect. The oil is apparently more active than the other fractions in the promotion of apoptosis, consistent with earlier published observations that gamma-tocopherol, in significant quantity in the oil, is also a strong promoter of apoptosis.

### Example 9

### Inhibition of 17-hydroxysteroid Dehydrogenase Type 1 by Pomegranate Fermented Juice and Pericarp Extracts and Supercritical Fluid Extracted Pomegranate Seed Oil.

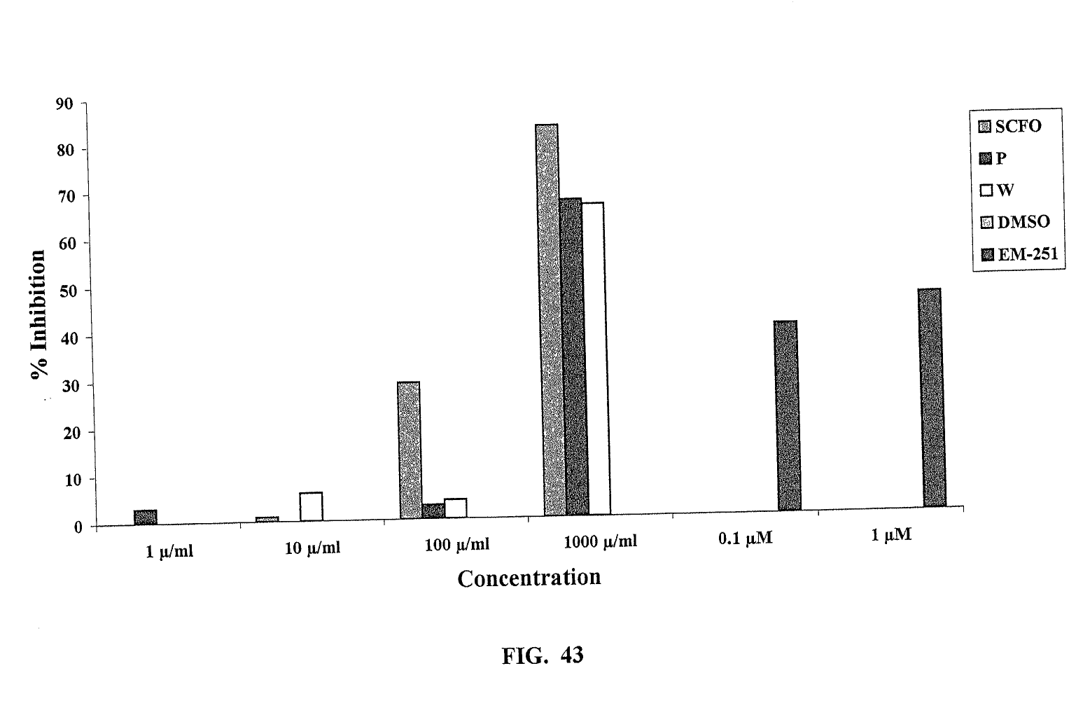

Extracts of pomegranate fermented juice (W) and pomegranate pericarp (P) were prepared according to the method of Schubert et al. ((1999) J Ethnopharmacol. 66(1):11-7), and pomegranate seed oil was obtained by supercritical fluid extraction (SCFO) utilizing CO₂ as a solvent. These three pomegranate fractions were tested at 1, 10, 100 and 1000 micrograms/ml as potential inhibitors of the enzyme 17-beta-hydroxysteroid dehdrogenase (HSD) type 1 utilizing the assay as previously reported by Luu-The et al ((1995) Biochem Mol Biol. 55(5-6):581-7). Specifically, the enzyme 17-beta-HSD type 1 was transfected into the cytosolic fraction of sonicated HEK-293 cells. A known inhibitor of 17-beta-HSD type 1, EM-251 (16-alpha-bromopropyl-estradiol) was employed as a positive control. The assay measures the transformation of estrone (E1) to 17-beta-estradiol (E2) utilizing thin-layer chromatography and quantification of C-14 labeled E1 and E2 using a Phosphor Imager, allowing for both the percent of transformation and the percent of inhibition. Results are summarized in FIG. 43. No significant inhibition was noted from any of the compounds at 1 and 10 micrograms/ml. All compounds caused inhibition at 1000 micrograms/ml, but only the oil caused inhibition at 100 micrograms/ml. These results demonstrate an additional mechanism by which pomegranate fractions inhibit the biosynthesis of active estrogen (E2) in vivo. This mechanism apparently complements the inhibition of aromatase, which leads to E2 by a different biosynthetic pathway. These results provide further evidence of the anti-estrogenic properties of pomegranate fractions and support the idea that a chemopreventive effect against estrogen-dependent breast cancer in vivo is likely. The differences between oil and pericarp or fermented juice extracts implies synergy between the aqueous and fatty phases of the pomegranate for cancer chemoprevention, especially estrogen-dependent cancer.

### Example 10

### Inhibition of Proliferation of Multiple Myeloma Cell Lines by Pomegranate Fermented Juice and Pericarp Extracts.

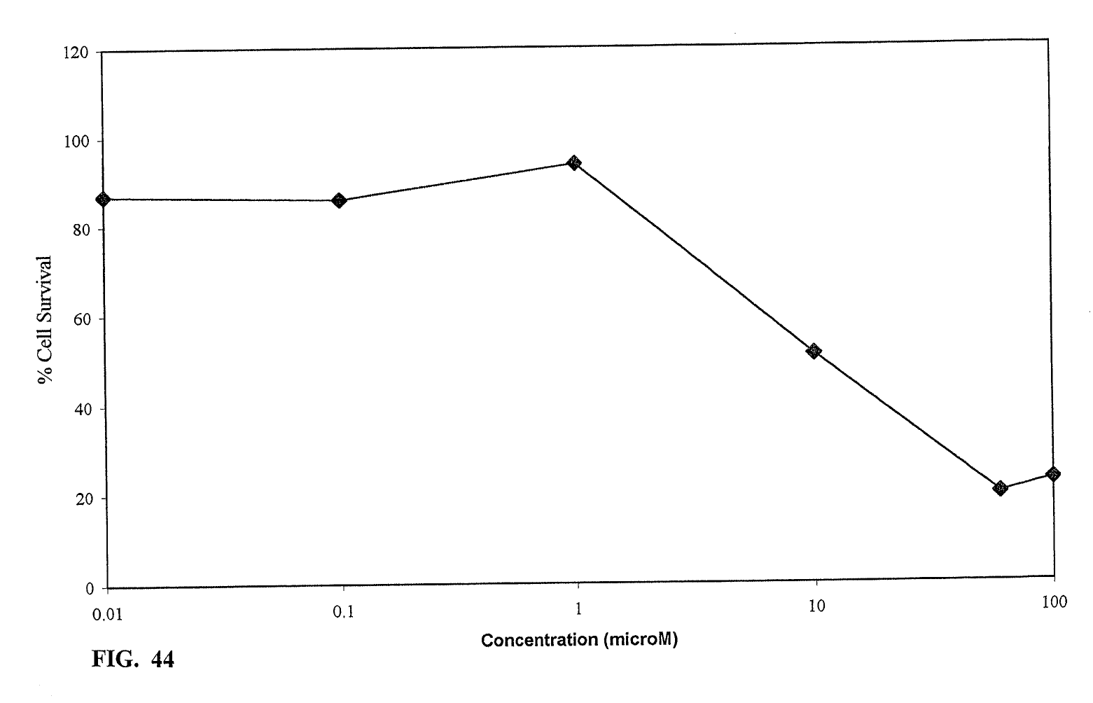

Three different human multiple myeloma cell lines[HS-Sultan (HSS), MM.1S and U266 as described in Gooding et al., (1999) J Haematol 106(3):669-81] were incubated for 24 hours with each of four different pomegranate fractions. The fractions were pomegranate fermented juice extract (W), pomegranate pericarp extract (P), supercritical CO₂ extracted pomegranate seed oil (SESCO) and ethanolic pomegranate seed cake extract (SEEE). The fractions were employed at mM quercetin equivalent concentrations as measured and described previously (Tedesco et al., (2000) J Nutr Biochem 11: 114-119). Inhibition of proliferation was assessed utilizing the MTT assay. Results are summarized in FIGS. 44-46. Significant inhibition of proliferation was noted in the U266 and HSS cell lines from between 10 micromolar to 100 micromolar concentrations, but not in the MM1S cells. These results demonstrate that human multiple myeloma cells are also subject to growth inhibition by pomegranate fractions. Solubility problems render the inhibition observed from the two oily fractions (SEEE and SESCO) somewhat equivocal. However, the inhibition by the aqueous fractions (P and W) was clear. Further studies will be needed to elucidate specific compounds within the extracts for whom the observed effects are dependent.

### Example 11

### Promotion of Differentiation in Human Breast Cancer Cells by a Pomegranate Fermented Juice Extract.

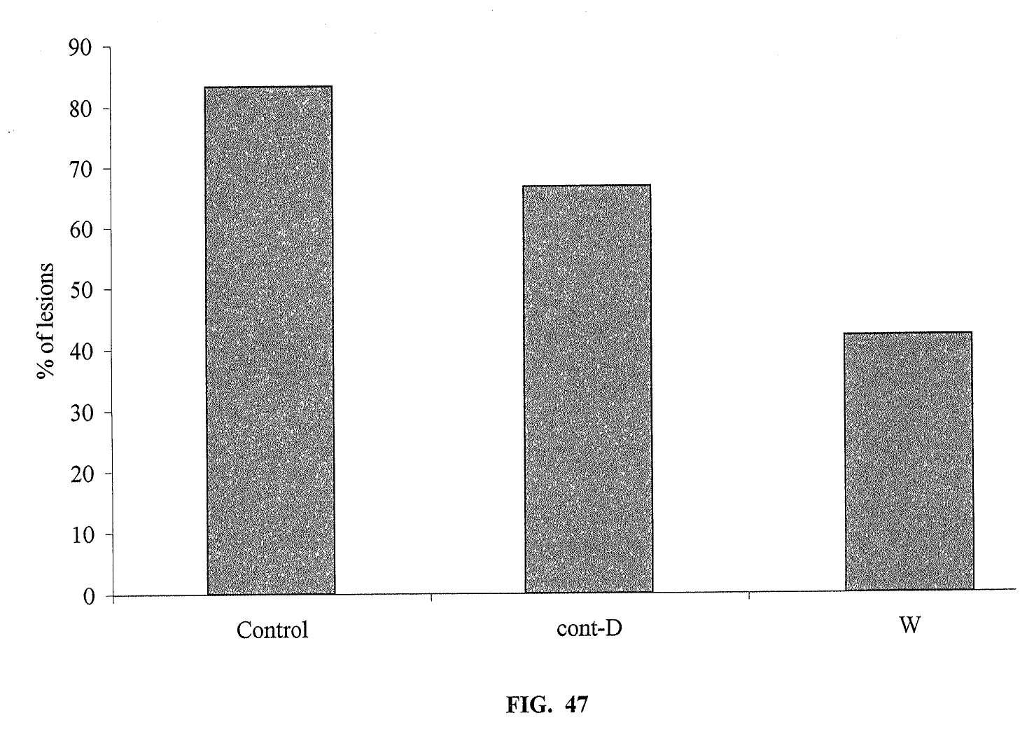

Mammary glands of BALB/c mice were placed in organ culture and treated with the carcinogen 7,12-dimethylbenz[a]anthracene (DMBA) to induce preneoplastic lesions (Mehta et al (1997) J. Nat. Cancer Inst. 89(3):212-219 and Mehta et al. (2000) J. Nat. Cancer Inst. 92(5):418-423). Results are summarized in FIG. 47. The experimental material (fermented pomegranate juice polyphenols) was added to the culture medium for 5 days of growth, and the number of neoplastic lesions evaluated in 25 glands. A 42% reduction was noted in the experimental group, compared to only a 20% reduction from a Vitamin D analog known as a differentiation inducer. In previous studies reported in the literature, there is excellent correlation between such experimental lesions and the development of tumors in athymic mice in which the cells from neoplastic lesions produced in this way are injected. These results suggest that the tested pomegranate fraction is able to reverse the cancer-promoting effect of a known carcinogen, apparently by promoting cell differentiation. Therefore, the tested material appears to be a novel cancer chemopreventive agent. Because the tested pomegranate extract, unlike most vitamin D analogs, has no known undesirable hypercalcemic side effect, it is particularly attractive. In an additional experiment using the same assay, a polyphenol fraction derived from a fermented juice extract exhibited 86% inhibition of tumors. This suggests that additional fractionation and purification of pomegranate derived materials is capable of producing efficacious chemopreventive agents.

### Example 12

### Pomegranate Fermented Juice and Pericarp Extracts Differentially Inhibit Proliferation of Cancer Cell Lines and Their Normal Counterparts.

Utilizing the Folin-Ciocalteu method (J. Nutr. Biochem., 11: 114, 2000), the concentration of total polyphenols in cold pressed pomegranate seed oil and in ethyl acetate extracts of pomegranate fermented juice and pericarp extracts (J. Ethnopharmacol., 66: 11, 1999) was quantified as millimolar equivalents of the flavonoid quercetin. The concentrations obtained were:

cold pressed pomegranate seed oil=4 mM eq. Quercetin;

pomegranate pericarip extract (P)=56 mM eq. Quercetin; and

pomegranate fermented juice extract (W)=133 mM eq. quercetin.

Selected concentrations of the three materials were incubated with cells, at 200,000 cells per well, for 24 h at 37° C. in the presence of a 5% CO₂ atmosphere. Cell lines employed were:

HeLa derived from a human cervix epitheliod carcinoma (Cancer Res. 12: 264, 1952);

HT-29 derived from a human colon adenocarcinoma (In Human tumor cells in vitro, pp. 115, J. Foght (ed.), Plenum Press, New York, 1975);

W138 human diploid cell line from normal embryonic lung tissue (Exp. Cell Res. 25: 585, 1961);

HPB-ALL from a human thymoma (Int. J. Cancer, 21:166, 1978); and

PBL peripheral blood lymphocytes isolated from healthy volunteers.

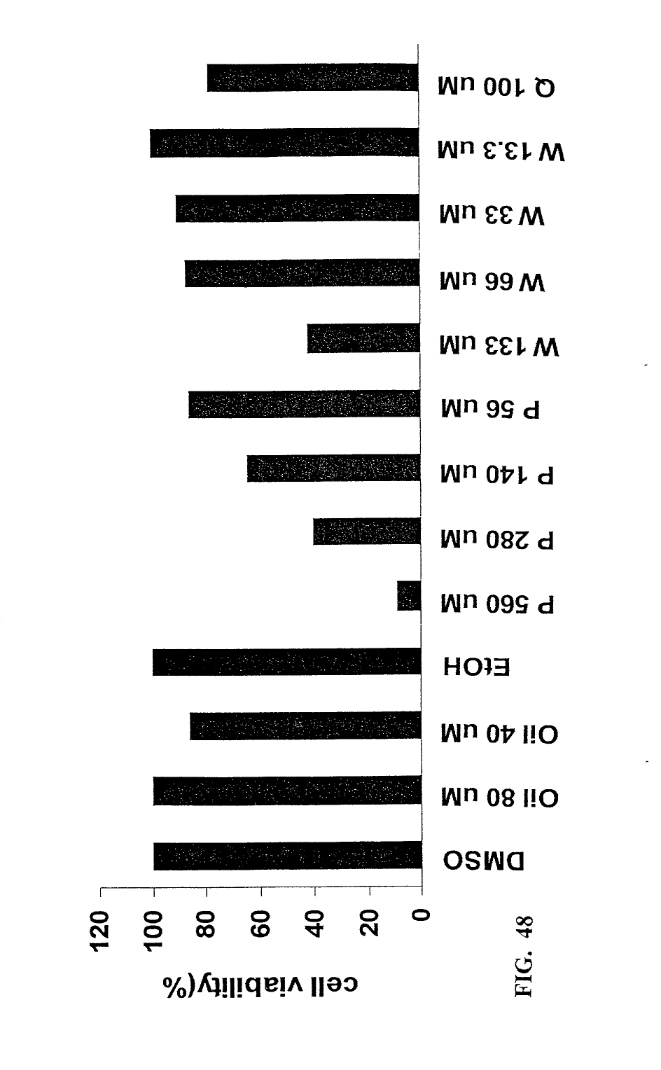

Results are summarized in FIGS. 48-51 and in table 1. The pericarp and fermented juice extracts significantly inhibited proliferation of all cancer cell lines in a dose dependent manner. Most sensitive to these fractions were the HPB-ALL cells, which were three times more sensitive than their normal counterpart, PBL cells. These findings suggest that the pomegranate extracts are specifically toxic to cancer cells, but not to normal cells. Because of solubility problems, the cytotoxicity of the oil was not adequately assessed. These findings further broaden the range of cancer cells whose growth is inhibited by extracts from pomegranate pericarp and fermented juice. These results are the first which quantify the total polyphenol content of assayed extracts and compare their activities to that of quercetin in a dose-dependent manner. In addition, the difference between the toxicity of the assayed fractions to cancer cells as opposed to normal cells is clearly demonstrated, suggesting that pomegranate extracts can be efficaciously employed as safe and effective cancer chemopreventive agents.

TABLE 1
Toxicity of pomegranate pericarp (P) and fermented juice (W)
extracts on selected cell lines expressed in mM equivalents
of quercetin.

| | IC50 (micro Molar) | | |
|---|---|---|---|
| Cell type | P | W | Oil |
| HT29 | <140 | 66-133 | >80 |
| WI38 | ±140 | ±66 | >80 |
| HPB-ALL | 5.6-14 | <13.2 | >80 |

### Example 13

### Production of an Elixir From a Mixture of Pomegranate Products.

FIG. 52 shows production steps in manufacture of a pharmaceutical composition including 30% dealcoholized concentrated pomegranate wine, 10% aqueous extract of pomegranate pericarp, and 60% seed cake extract. The 120 ml of elixir represents 1000 doses of an elixir for women which could be delivered, for example as gel-caps. The elixir is expected to have beneficial effects in climacteria as well as to offer protection against development of breast cancer and to be beneficial in treating breast cancer. Raw materials are 1440 Kg of whole pomegranates and 1099 kg of pomegranate seeds. The pomegranates are initially processed into juice and pericarp. The juice is then fermented and distilled. The dealcoholized wine is then reduced to a 36 kg. Concentrate containing approximately 20% total solids. The pericarp is subjected to an aqueous extraction which produces a pericarp extract containing approximately 20% total solids. The seeds are “dry cleaned” or solvent extracted to produce a seed cake which is further extracted with ethanol. The resultant seed cake extract becomes a component of the elixir.

FIG. 53 shows production steps in manufacture of a pharmaceutical composition including 70% dealcoholized concentrated pomegranate wine, 10% aqueous extract of pomegranate pericarp, and 20% seed cake extract. The 120 ml of elixir represents 1000 doses of an elixir for men which could be delivered, for example, as gel-caps. The elixir is expected to have beneficial effects preventing benign prostatic hyperplasia (BPH) and/or prostate cancer. The production process is essentially as described for the elixir for women.

### Example 14

### Production of Gel-caps From Pharmaceutical Compositions According to the Present Invention.

As detailed hereinabove, pharmaceutical compositions of the present invention may be provided in a wide variety of physical forms. One of these forms is gel-caps. Production of gel-caps typically includes the following steps:

1) Obtaining concentrated fermented juice and concentrated aqueous pericarp extracts.

2) Mixing the two components together, for example in a 9:1 ratio (fermented juice: pericarp).

3) Submitting these component extracts (together or individually) to supercritical fluid extraction using CO2 as a solvent with an ethanol modifier to obtain a polyphenol fraction. Suspending this polyphenol fraction in pomegranate seed oil in a ratio of, for example, 1:100 (polyphenol fraction: seed oil) to prepare the chemopreventive/phytoestrogen supplement. The prepared elixir may be encapsulated, for example in soft gel capsules.

### Example 15

### Inhibition of Colony Formation by Pomegranate Seed Oil

In order to determine the potential utility of pomegranate seed oil in treatment of breast cancer, a colony forming assay using the MDA-MB-435 breast cancer cell line was performed. Oil was dissolved in DMSO first at 1:10 and incubated at 37 degrees centigrade for 1 hour then further diluted 1:10 in 6.25 mg/ml of BSA and further incubated at 37 degrees centigrade for 1 hour. Tested pomegranate seed oil (Sde Eliahu 2000 organic “cold” pressed pomegranate seed oil) inhibited colony formation 50 to 100% as compared to corn oil or DMSO controls. Inhibition was observed at concentrations from 10 to 100 micrograms/ml. These results indicate that the tested oil has potent biological activity including the ability to prevent proliferation of hormonally dependent cancer cells.

### Example 16

### Selective Modulation of Estrogen Receptors in vitro

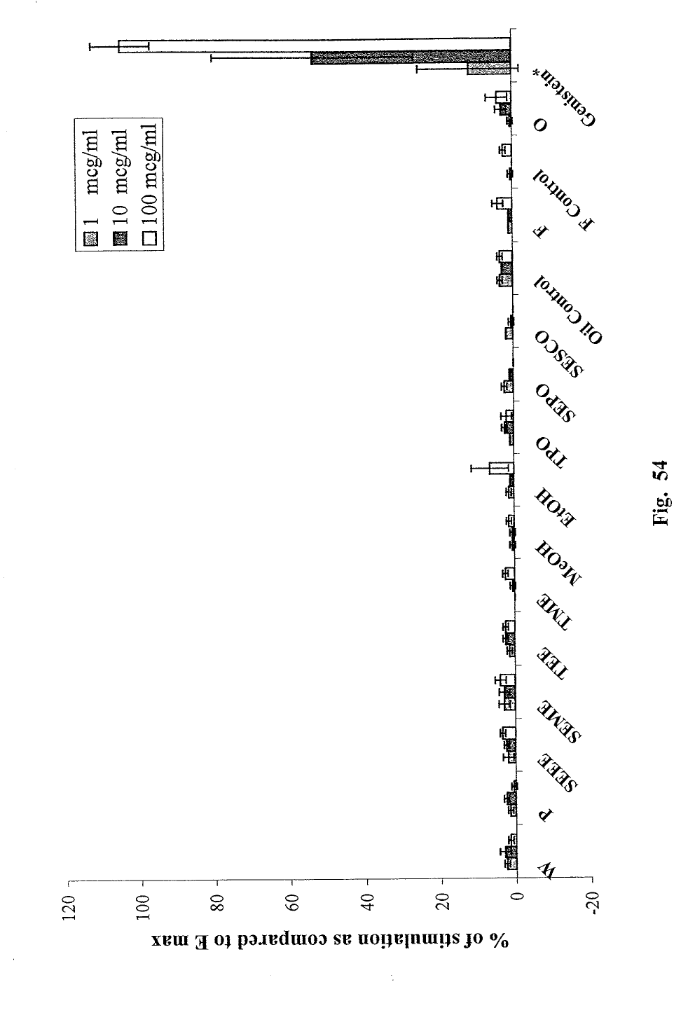

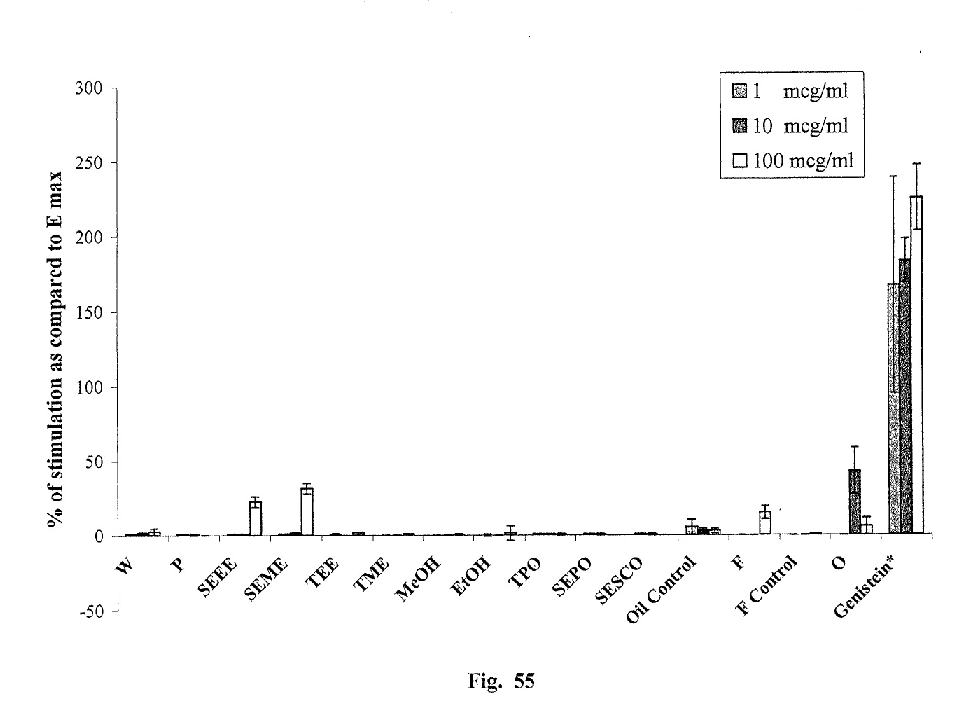

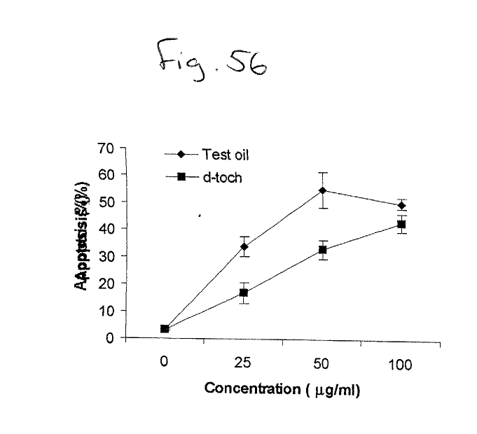

In order to determine the ability of various pomegranate products to selectively modulate estrogen receptors, an ER alpha and beta stimulation assays were performed.) Results, expressed as a percentage of stimulation relative to EMAX (beta estradiol) are summarized in FIGS. 54 and 55. Blue bars represent 1 mcg/ml, red bars represent 10 mcg/ml and yellow bars represent 100 mcg/ml. W indicates fermented pomegranate juice polyphenols; P indicates pomegranate pericarp polyphenols; SEEE indicates Sde Eliahu ethanolic; SEME indicates Sde Eliahu methanolic pomegranate seed extract; TEE indicates Turkish c pomegranate seed extract ethanolic pomegranate seed extract; TME indicates Turkish methanolic pomegranate seed extract; MeOH indicates methanol; EtOH indicates ethanol; TPO indicates Turkish pressed oil; SEPO indicates Sde Eliahu pressed oil; SESCO indicates Sde Eliahu supercritical carbon dioxide extracted oil; and F indicates pomegranate flower ethanolic extract. FIG. 54 indicates estrogen alpha activity and FIG. 55 represents estrogen beta activity. Ethanolic and methanolic pomegranate seed extracts from fruit grown in the Sde Eliahu groves (Israel) exhibited significant beta estrogen activity bit almost no alpha estrogen activity. This data, together with the yeast estrogen screen data presented in example 1 indicate selective estrogen receptor modulation by the tested fraction or components thereof. Presence of SERMs in the assayed fraction is also consistent with proliferation/apoptosis data for HL-60 cells as detailed hereinabove.

### Example 17

### Inhibition of Apoptosis by Pomegranate Seed Oil

In order to determine the antiapoptotic effect of pomegranate seed oil, the oil was assayed in a cell culture system. An estrogen receptor negative human metastatic breast cancer cell line (MDA-MB-435) was maintained in minimal medium (MEM; Hyclone Laboratories, Logan, Utah) in T-75 flasks (Coring Plastic ware, Corning, N.Y.). Monolayers were supplemented with 5% fetal calf serum (FBS; Hyclone Laboratories), 2 mM glutamine, 1% (vol/vol) 100× non-essential amino acids, 2% (vol/vol) 100× MEM vitamin solution, 1 mM sodium pyruvate (Sigma Chemical Co), 100 IU/ml penicillin, 100 micrograms/ml streptomycin (Gibco BRL Products, Gaithersburg, Md.). Cultures were incubated in a humidified atmosphere of 5% CO₂-95% air at 37° C. Cells were harvested by 0.25% trypsin-1 mM ethylenediaminetetra-acitic acid (EDTA). Cultures were routinely examined to verify absence of mycoplasma contamination. Exponentially growing cells were plated at 3×10⁴ cells/well in 12 well plates and allowed to attach overnight, then treated with test oil, gamma-tocopherol, fish oil, corn oil [25, 50, and 100 microgram/ml in a concentration of 0.1% (vol/vol) DMSO] in reduced serum (2% serum) for 3 days.

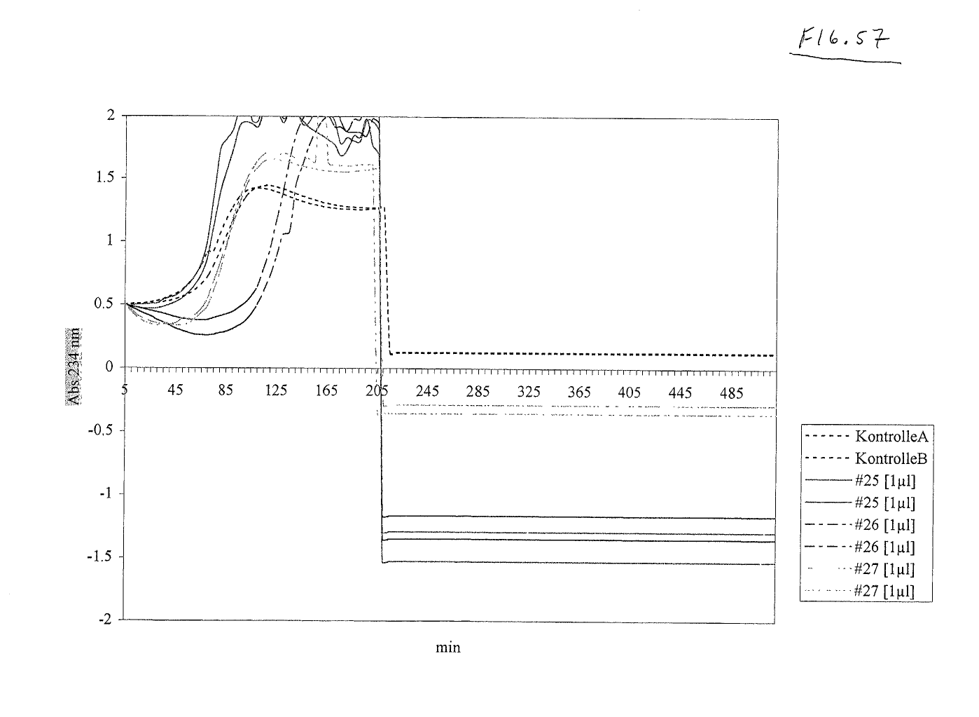

Morphological analyses of apoptosis by DAPI staining was conducted according to published methods (Yu, W., et al. (1999) Cancer Res. 59(4): p. 953-61). Apoptosis was assessed, based on nuclear morphology, using fluorescent DNA binding dye 4,6-diamidino-2-phenylindole dihydrochloride (DAPI, Boehringer Mannheim, Indianapolis, Ind.). Treated cells were collected, centrifuged, resuspended in 2 microgram/ml of DAPI in 100% methanol and viewed under 400× magnification using a fluorescent microscope (Zeiss ICM 405) with a 487701 filter. Apoptotic data, summarized in FIG. 56 are reported as percent apoptosis, determined by counting apoptotic cells and non-apoptotic cells % apoptosis=[apoptotic cells/(apoptotic cell+non-apoptotic cells)×100]. Cells containing clearly condensed nuclear chromatin or cells exhibiting fragmented nuclei were scored as apoptotic cells. For each sample, a minimum of 3 counts involving a minimum of 100-200 cells/count were scored. Apoptotic data are presented as mean ±SD for three independently performed experiments. Reagents for morphological analyses of apoptosis were purchased from Boehringer Mannheim (Indianapolis, Ind.). These results indicate that the tested oil significantly retards apoptosis and that this effect is independent of the estrogen receptor.

### Example 18

### Reduction in LDL Oxidation by Pomegranate Seed Oil

In order to determine the effect of pomegranate seed oil on LDL oxidation samples #25 (Sde Eliahu Israel) pressed oil; #26 indicates (CO2 supercritical fluid extract of Sde Eliahu pressed oil) and # 27 (Turkish pressed oil) were compared to olive oil (Kontrolle).

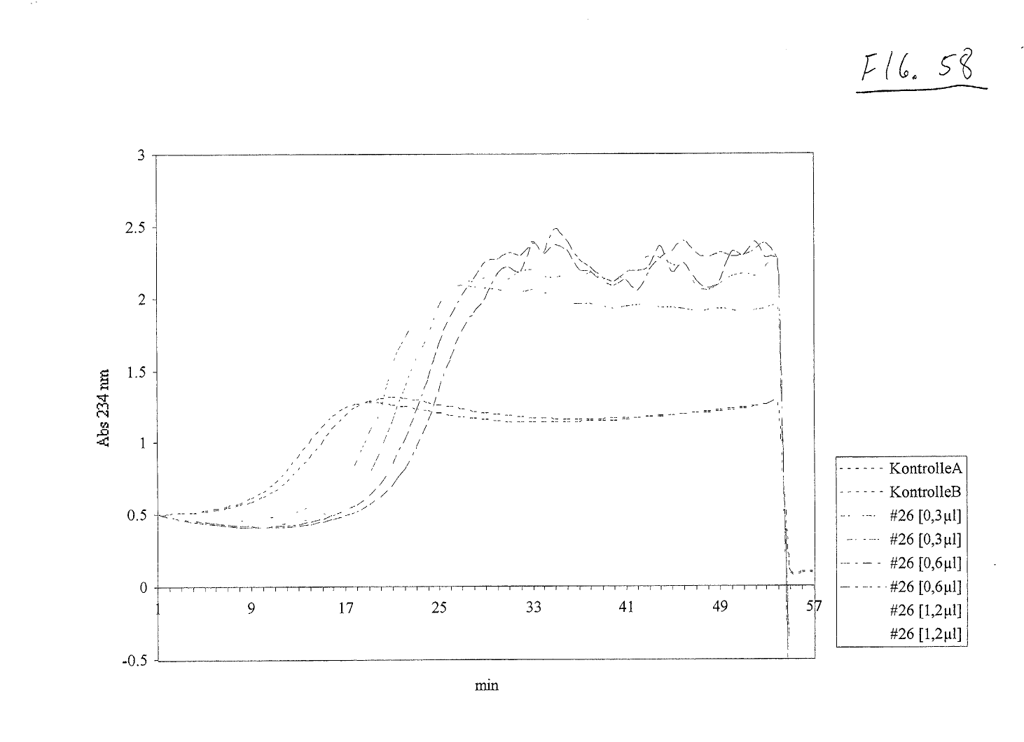

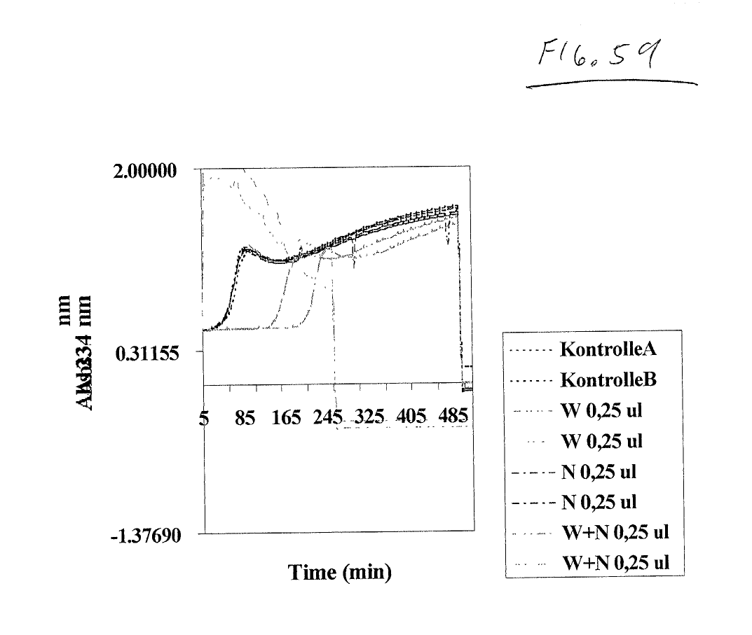

The assay was performed by published methods (Dittrich et al. (1999) Exp. Clin. Endocrinol. Diabetes 107(1):53-7). A reduction in absorbance at 234 nanometers relative to olive oil indicates inhibition of LDL oxidation. Results, summarized in FIG. 57, indicate that samples 26 and 27 significantly inhibit LDL oxidation. FIG. 58 shows that the inhibition is concentration dependent for sample 26. These results imply a role for pomegranate seed oil, or fractions thereof, in regulating or reducing blood cholesterol levels.

### Example 19

### Synergistic Reduction in LDL Oxidation by Pomegranate Fractions

Pomegranate fermented juice polyphenol fraction (W) and unsaponifiable fraction of pomegranate seed oil (N) were assayed as in Example 18. Results are summarized in FIG. 59. Significant synergy between W and N is observed.

### Example 20

### Specific Estrogen Receptor Agonism

In order to determine the specificity of Estrogen receptor alpha and beta agonism, samples were tested in a hepatoma cell line transfected with ER alpha/beta galactosidase and ER beta/beta galactosidase constructs. Agonism caused increased beta galactosidase activity which is measured in arbitrary units. Results are summarized in Table 2. Methanolic seed cake extract was nearly four times more active on ER alpha than on ER beta. Cold pressed oil from seeds was twice as active on ER beta as on ER alpha. These results indicate that there are SERMs present in pomegranate seeds.

TABLE 2
Beta Galactosidase activity in response to pomegranate fractions.

| | ERalpha/beta | ERbeta/beta |
|---|---|---|
| Sample | galactosidase | galactosidase |
| Methanolic | 2016 | 534 |
| seed cake | | |
| extract | | |
| (Turkey) | | |
| Cold pressed | 250 | 500 |
| oil from seeds | | |
| (Sde Eliahu; | | |
| Israel) | | |

### Example 21

### Identification of Biologically Active Components in Pomegranate Peel Extract

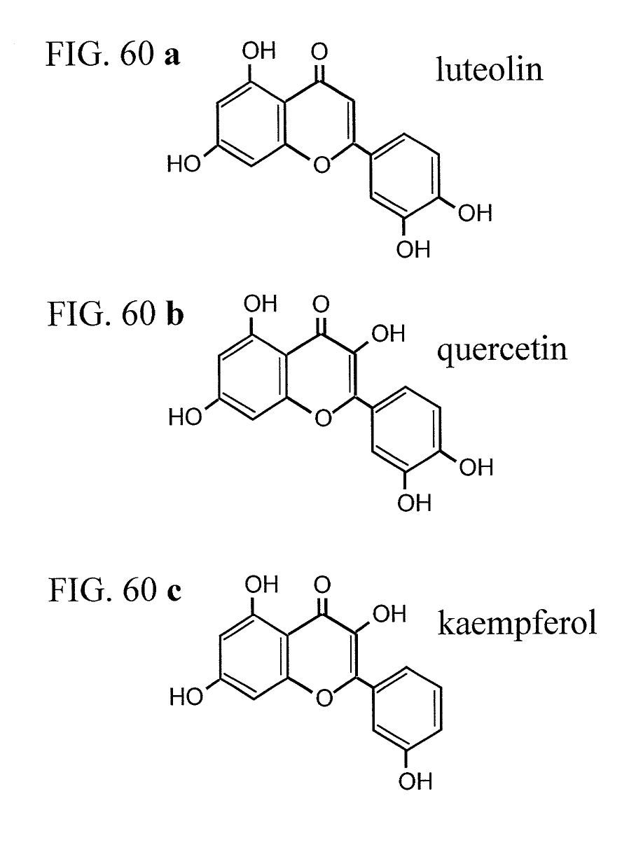

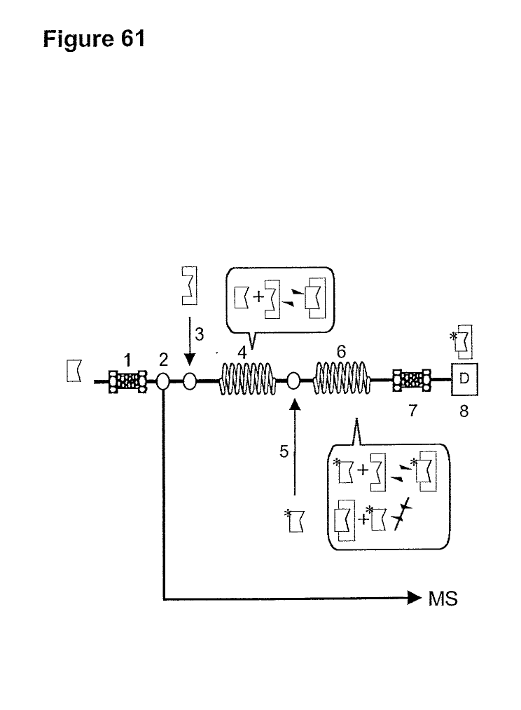

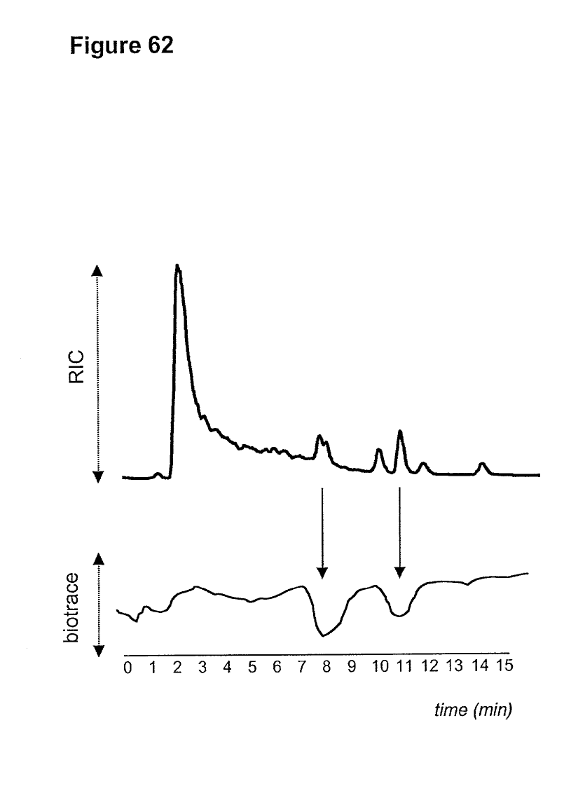

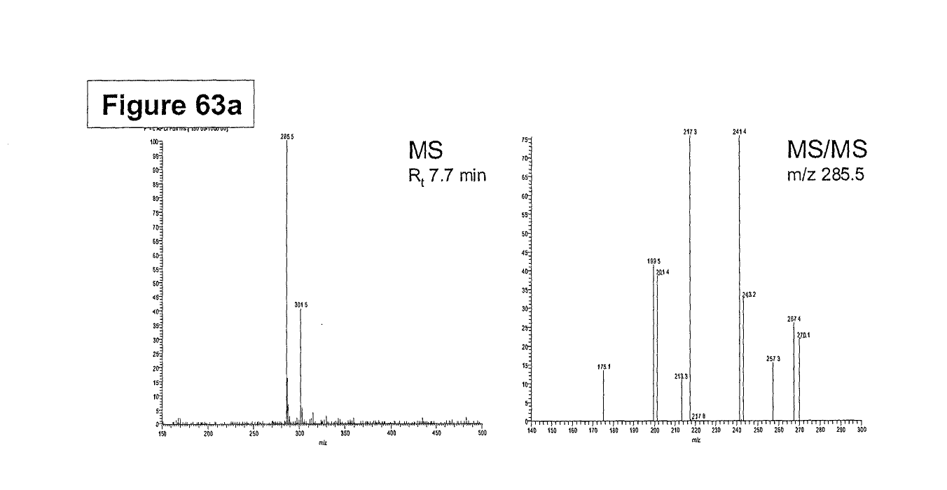

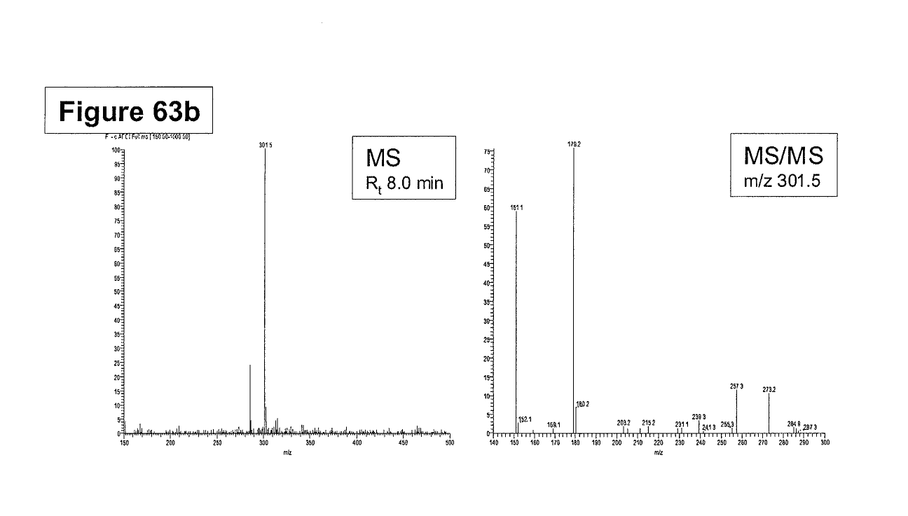

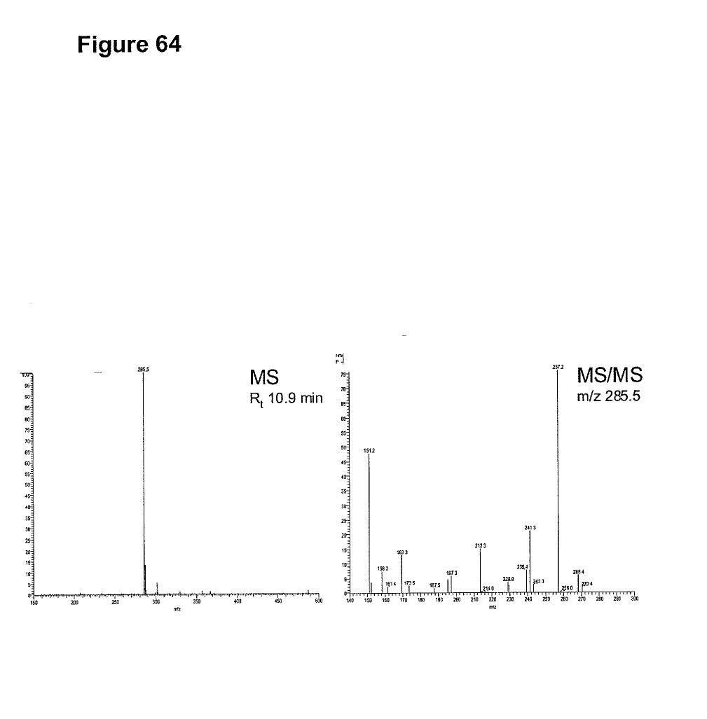

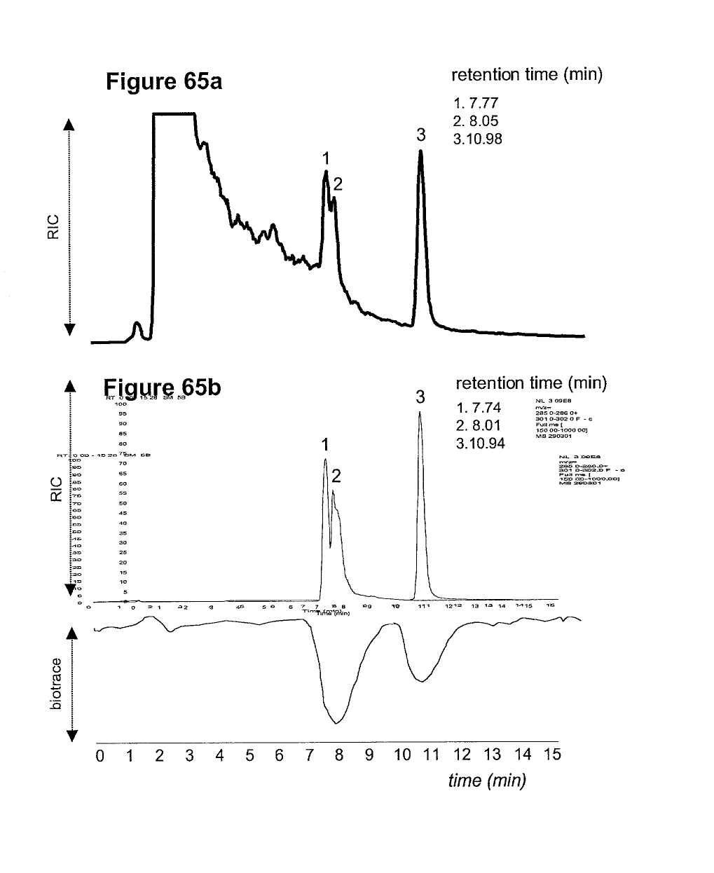

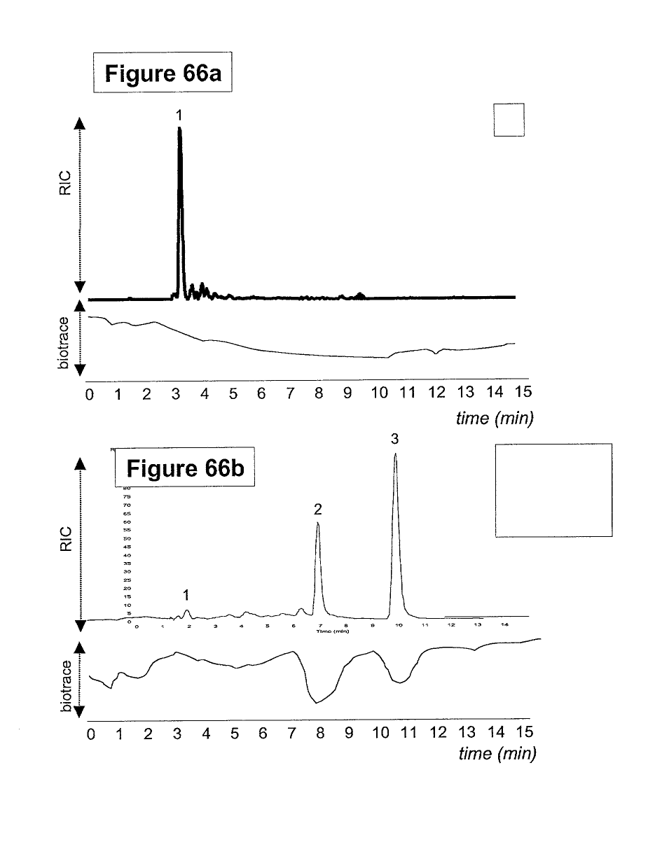

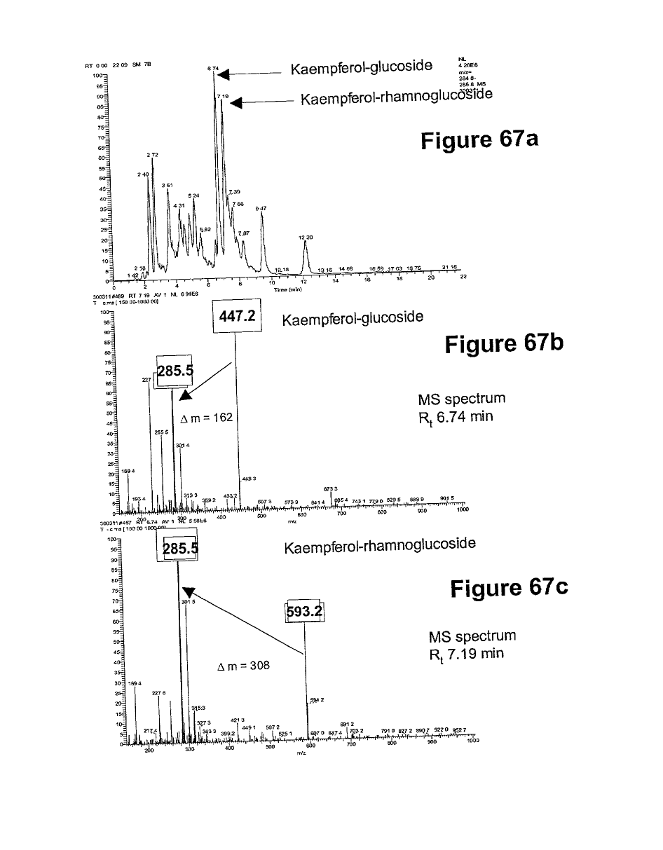

In order to identify biologically active components in pomegranate peel extract, separation and purification was carried out (FIG. 61). FIG. 61 is a flow scheme of on-line characterization screening of pomegranate peel extract with 1 representing a Biogradient; 2 representing a Flow split with 50 μl/min diverted to biochemical detection and 1150 μl/min to routed Mass Spectroscopy (MS). 3 represents addition of ER beta via a superloop and 4 represents a reaction coil. 5 indicates addition of coumestrol and 6 represents an additional reaction coil. 7 depicts a restricted access column and 8 represents a fluorescence detector. Fluorescence indicates biological activity in binding the ER beta receptor. FIG. 62 shows results of an analysis of acid hydrolyzed pomegranate peel extract. The top trace indicates total ion current full scan MS as a function of time. The bottom trace indicates fluorescence detection readout of the same sample. Top and bottom traces are corrected for time delay. These results correlate the biological activity to indicated peaks in the MS trace. FIGS. 63a and b depict MS and MS/MS spectra corresponding to the first peak in the bottom trace of FIG. 62. FIG. 63a illustrates MS and MS/MS spectra of co-eluting compound m/z 285.5. FIG. 63b illustrates MS and MS/MS spectra of co-eluting compound m/z 301.5. Time difference between MS spectra A and B equals 0.3 min. These data resolve the 8 minute peak of FIG. 62 into two separate peaks with elution times of 7.7 and 8.0 minutes respectively. FIG. 64 depicts MS and MS/MS spectra corresponding to the first portion of the second peak (at 11 minutes) in the biochemical trace of FIG. 62. This fraction has MS and MS/MS spectra of compound with m/z 285.5. FIGS. 65a and b illustrate the correlation of bioactivity to reference compounds: luteolin (1), quercetin (2) and kaempferol (3). FIG. 65a is an MS ion current trace of hydrolyzed pomegranate peel extract (m/z 285.3 and 301.5). In FIG. 65b the top trace is an MS ion current trace of a reference solution containing 260 μM luteolin, 280 μM quercetin and 460 μM kaempferol (m/z 285.3 and 301.5). The bottom trace is a bioactivity trace of the reference solution. This bioactivity trace closely resembles that of FIG. 62, indicating that the assayed peel extract almost certainly contains luteolin, quercetin and kaempferol (FIG. 60). FIGS. 66a and b compare original pomegranate peel extract and acid hydrolyzed pomegranate peel extract. The acid hydrolysis simulates digestion in a human stomach. FIG. 66a shows analysis of the original pomegranate peel extract. The top trace is an MS/MS trace indicating the presence of (1) glycosylated luteolin m/z199.5. The bottom trace is a biochemical trace. FIG. 66B presents an analysis of the acid hydrolyzed pomegranate peel extract. The top trace is an MS trace indicating the presence of (1) glycosylated luteolin (2) luteolin (m/z 285.5) (3) kaempferol (m/z 285.5). The bottom trace is a biochemical trace. Taken together, these data indicate that the process of hydrolysis enriches for quercetin (2) and kaempferol while decreasing the concentration of luteolin (1) and degycosylating the compounds. This deglycosylation enhances the biological activity. FIGS. 67a, b and c constitute a further characterization of glycosylated phytoestrogens in pomegranate peel extract. FIG. 67a shows the MS ion current for m/z 285.5 (kaempferol/luteolin). FIG. 67b shows MS spectrum kaempferol-glucoside (Rₜ 6.74 min). FIG. 67c shows MS spectrum kaempferol-rhamnoglucoside (Rₜ 7.19 min). MS/MS data dependent scanning, original extract. Gradient 0-20% in 20 min. Minimum/maximum flow rate in Biogradient respectively 0.37 and 0.5 ml/min. These data indicate that glycosylated luteolin quercetin and kaempferol which have lower biological activity than their deglycosylated counterparts are most likely de-glycosylated after ingestion by a human subject. This deglycosylation serves to increase biological activity.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A cancer chemopreventive mixture, the mixture comprising:
(a) a pomegranate seed oil product; and
(b) a pomegranate juice product.

2. The mixture of claim 1, further comprising:
(c) a pomegranate peel product.

3. The mixture of claim 1, wherein said pomegranate seed oil product is the result of a process selected from the group consisting of expeller pressing, supercritical fluid extraction with carbon dioxide, and lyophilization.

4. The mixture of claim 1, wherein said pomegranate seed oil product is produced from a material selected from the group consisting of pomegranate seeds and pomegranate seed cake.

5. The mixture of claim 1, wherein said pomegranate seed oil product is selected from the group consisting of pomegranate seed oil and a non saponifiable fraction thereof.

6. The mixture of claim 1, wherein said pomegranate juice product comprises at least one item selected from the group consisting of pomegranate juice, fermented pomegranate juice, dried pomegranate juice, dried fermented pomegranate juice, partially fermented pomegranate juice, partially dried pomegranate juice, partially fermented partially dried pomegranate juice, reduced pomegranate juice, partially reduced pomegranate juice and lyophylysates thereof.

7. The mixture of claim 1, wherein the mixture is provided in a form selected from the group consisting of a liquid, a powder, granules, a tablet, a capsule, a gel-cap, an ointment, a cream, a chewing gum, a food, a candy, an emulsion and a suppository.

8. The mixture of claim 1, wherein the cancer is a hormone dependent cancer.

9. The mixture of claim 8, wherein the hormone dependent cancer is selected from the group consisting of breast cancer and prostate cancer.

10. The mixture of claim 2, wherein said pomegranate peel product is selected from the group consisting of pomegranate peel residue present in pomegranate juice as a result of a juicing process, an aqueous extract of pomegranate peel, an alcohol extract of pomegranate peel, an extract performed with an organic solvent which is not an alcohol, and a supercritical fluid extract of pomegranate peel.

11. The mixture of claim 2, wherein said pomegranate peel product contains at least one item selected from the group consisting of quercetin, kaempferol, luteolin, derivatives thereof and combinations thereof.

12. The mixture of claim 10, wherein said supercritical fluid extract is conducted with at least one fluid selected from the group consisting of CO₂, ethanol, methanol and water.

13. A pharmaceutical composition, the composition comprising physiologically active amounts of:
(a) a pomegranate seed oil product;
(b) a pomegranate juice product; and
a pharmaceutically acceptable carrier.

14. The composition of claim 10, further comprising:
(c) a pomegranate peel product.

15. The composition of claim 10, wherein said pomegranate seed oil product is the result of a process selected from the group consisting of expeller pressing, supercritical fluid extraction with carbon dioxide, and lyophilization

16. The composition of claim 10, wherein said pomegranate seed oil product is produced from a material selected from the group consisting of pomegranate seeds and pomegranate seed cake.

17. The composition of claim 10, wherein said pomegranate seed oil product is selected from the group consisting of pomegranate seed oil and a non saponifiable fraction thereof.

18. The composition of claim 10, wherein said pomegranate juice product comprises at least one item selected from the group consisting of pomegranate juice, fermented pomegranate juice, dried pomegranate juice, dried fermented pomegranate juice, partially fermented pomegranate juice, partially dried pomegranate juice, partially fermented partially dried pomegranate juice, reduced pomegranate juice, partially reduced pomegranate juice, pomegranate peel residue and lyophylysates thereof.

19. The composition of claim 10, wherein the composition is provided in a form selected from the group consisting of a liquid, a powder, granules, a tablet, a capsule, a gel-tab, an ointment, a cream, a chewing gum, a food, a candy, an emulsion and a suppository.

20. The composition of claim 13, wherein said pomegranate peel product is selected from the group consisting of pomegranate peel residue present in pomegranate juice as a result of a juicing process, an aqueous extract of pomegranate peel, an alcohol extract of pomegranate peel, an extract performed with an organic solvent which is not alcohol, and a supercritical fluid extract of pomegranate peel.

21. The composition of claim 13, wherein said pomegranate peel product contains at least one item selected from the group consisting of quercetin, kaempferol, luteolin, derivatives thereof and combinations thereof.

22. The composition of claim 20, wherein said supercritical fluid extract is conducted with at least one item selected from the group consisting of CO₂, ethanol, methanol and water.

23. The pharmaceutical composition of claim 10, wherein the pharmaceutical composition is efficaciously employed for treatment of a medical condition.

24. The pharmaceutical composition of claim 10, wherein said medical condition is selected from the group consisting of cancer, Alzheimer's disease, climacteria, benign prostatic hyperplasia and estrogen deficiency.

25. The composition of claim 24, wherein the cancer is a hormone dependent cancer.

26. The composition of claim 25, wherein the hormone dependent cancer is selected from the group consisting of breast cancer and prostate cancer.

27. The composition of claim 25, wherein said treatment is selected from the group consisting of a prophylactic treatment, a palliative treatment and a therapeutic treatment.

28. The composition of claim 10, wherein the physiologic activity results from inhibition of an enzyme selected from the group consisting of aromatase and 17-beta-hydroxysteroid dehdrogenase (HSD) type 1.

29. The composition of claim 10, wherein physiologically active ingredients comprise dealcoholized concentrated pomegranate wine, aqueous extract of pomegranate pericarp, and seed cake extract.

30. The composition of claim 29, wherein physiologically active ingredients comprise approximately 70% dealcoholized concentrated pomegranate wine, approximately 10% aqueous extract of pomegranate pericarp, and approximately 20% seed cake extract.

31. The composition of claim 29, wherein physiologically active ingredients comprise approximately 30% dealcoholized concentrated pomegranate wine, approximately 10% aqueous extract of pomegranate pericarp, and approximately 60% seed cake extract.

32. A novel selective estrogen receptor modulator (SERM) derived from pomegranate, the novel SERM comprising at least one item selected from the group consisting of:
(a) a pomegranate seed product;
(b) a pomegranate juice product; and
(c) a pomegranate peel product.

33. The SERM of claim 32, wherein said pomegranate seed product is the result of a process selected from the group consisting of cold pressing, expeller pressing, supercritical fluid extraction with carbon dioxide, and lyophilization.

34. The SERM of claim 32, wherein said pomegranate seed product includes at least one item selected from the group consisting of pomegranate seed oil, an unsaponified fraction thereof and an alcohol extract of pomegranate seed cake.

35. The SERM of claim 32, wherein said alcohol employed to produce said alcohol extract includes at least one item selected from the group consisting of methanol, ethanol, propanol and butanol.

36. The SERM of claim 32, wherein said pomegranate juice product includes at least one item selected from the group consisting of pomegranate juice, fermented pomegranate juice, dried pomegranate juice, dried fermented pomegranate juice, partially fermented pomegranate juice, partially dried pomegranate juice, partially fermented partially dried pomegranate juice, reduced pomegranate juice, partially reduced pomegranate juice and lyophylysates thereof.

37. The SERM of claim 32, wherein the mixture is provided in a form selected from the group consisting of a liquid, a powder, granules, a tablet, a capsule, a gel-cap, an ointment, a cream, a chewing gum, a food, a candy, an emulsion and a suppository.

38. The SERM of claim 32, wherein the mixture has utility for a purpose selected from the group consisting of retardation of the development of cancer, cancer treatment, hormone replacement therapy, retarding the aging process, skin treatment, relief of menopausal dryness, and relief of dyspareunia.

39. The SERM of claim 38, wherein said cancer is a hormone dependent cancer.

40. The SERM of claim 32, wherein said pomegranate peel product is selected from the group consisting of pomegranate peel residue present in pomegranate juice as a result of a juicing process, an aqueous extract of pomegranate peel, an alcohol extract of pomegranate peel, an extract performed with an organic solvent which is not alcohol, and a supercritical fluid extract of pomegranate peel.

41. The SERM of claim 32, wherein said pomegranate peel product contains at least one item selected from the group consisting of quercetin, kaempferol, luteolin, derivatives thereof and combinations thereof.

42. The SERM of claim 40, wherein said supercritical fluid extract is conducted with at least one item selected from the group consisting of CO₂, ethanol, methanol and water.

43. A method for selectively modulating an estrogen receptor, the method comprising the steps of:
(a) providing at least one item selected from the group consisting of
(i) a pomegranate seed product;
(ii) a pomegranate juice product; and
(iii) a pomegranate peel product; and
(b) allowing a SERM contained in said at least one item to selectively modulate an estrogen receptor.

44. The method of claim 43, further comprising the step of:
(c) combining at least two of said at least one item.

45. The method of claim 41, wherein said step of combining includes combining at least three of said at least one item.

46. The method of claim 43, wherein said step of providing said pomegranate seed product includes a process selected from the group consisting of expeller pressing, supercritical fluid extraction with carbon dioxide, and lyophilization.

47. The method of claim 43, wherein said step of providing said pomegranate seed product includes at least one process selected from the group consisting of pressing pomegranate seeds to produce pomegranate seed oil and extracting pomegranate seed cake with an alcohol.

48. The method of claim 44, wherein said alcohol employed for said extraction includes at least one item selected from the group consisting of methanol, ethanol, propanol and butanol.

49. The method of claim 43, wherein said step of providing said pomegranate juice product includes at least one process selected from the group consisting of extracting juice from a pomegranate, fermenting pomegranate juice, drying pomegranate juice, drying fermented pomegranate juice, partially fermenting pomegranate juice, partially drying pomegranate juice, partially fermenting partially dried pomegranate juice, reducing pomegranate juice, partially reducing pomegranate juice and lyophylysation of a product of any process belonging to the group.

50. The method of claim 43, wherein the step of allowing said SERM to selectively regulate an estrogen receptor includes the sub-step of providing the serm in a form selected from the group consisting of a liquid, a powder, granules, a tablet, a capsule, a gel-cap, an ointment, a cream, a chewing gum, a food, a candy, an emulsion and a suppository.

51. The method of claim 43, wherein said step of allowing said SERM to selectively modulate an estrogen receptor has utility for a purpose selected from the group consisting of retardation of the development of cancer, cancer treatment, hormone replacement therapy, retarding the aging process, skin treatment, relief of menopausal dryness, and relief of dyspareunia.

52. The method of claim 48, wherein said cancer is a hormone dependent cancer.

53. The method of claim 43, wherein said pomegranate peel product is selected from the group consisting of pomegranate peel residue present in pomegranate juice as a result of a juicing process, an aqueous extract of pomegranate peel, an alcohol extract of pomegranate peel, an extract performed with an organic solvent which is not alcohol, and a supercritical fluid extract of pomegranate peel.

54. The method of claim 43, wherein said pomegranate peel product contains at least one item selected from the group consisting of quercetin, kaempferol, luteolin, derivatives thereof and combinations thereof.

55. The method of claim 53, wherein said supercritical fluid extract is conducted with at least one item selected from the group consisting of CO₂, ethanol, methanol and water.

56. A novel method of producing 17 alpha estradiol, the method comprising the steps of:
(a) extracting oil from pomegranate seeds; and
(b) purifying the 17 alpha estradiol therefrom.

57. A novel method of producing gamma tocopherol, the method comprising the steps of:
(a) extracting oil from pomegranate seeds; and
(b) purifying the gamma tocopherol therefrom

58. A novel method of producing campesterol, the method comprising the steps of:
(a) extracting oil from pomegranate seeds; and
(b) purifying the campesterol therefrom.

59. A novel method of producing a composition containing a physiologically active amount of 17 alpha estradiol, the method comprising the step of extracting oil from pomegranate seeds, said oil containing the physiologically active amount of 17 alpha estradiol

60. The method of claim 59, wherein said step of extracting includes at least one process selected from the group consisting of cold pressing, expeller pressing, supercritical fluid extraction with carbon dioxide, and lyophilization.

61. A novel method of producing a composition containing a physiologically active amount of gamma tocopherol, the method comprising the step of extracting oil from pomegranate seeds; said oil containing the physiologically active amount of gamma tocopherol.

62. The method of claim 61, wherein said step of extracting includes at least one process selected from the group consisting of cold pressing, expeller pressing, supercritical fluid extraction with carbon dioxide, and lyophilization.

63. A novel method of producing a composition containing a physiologically active amount of campesterol, the method comprising the step extracting oil from pomegranate seeds, said oil containing the physiologically active amount of campesterol.

64. The method of claim 63, wherein said step of extracting includes at least one process selected from the group consisting of cold pressing, expeller pressing, supercritical fluid extraction with carbon dioxide, and lyophilization.

65. A novel method for use of pomegranate seed oil, the method comprising the step of administering the pomegranate seed oil to a subject to exert an effect on a physiologic process.

66. The method of claim 65, wherein the oil is extracted by at least one process selected from the group consisting of cold pressing, expeller pressing, supercritical fluid extraction with carbon dioxide, and lyophilization.

67. The method of claim 65, wherein said subject is a selected from the group consisting of an animal and a human.

68. The method of claim 65, wherein said physiologic process is selected from the group consisting of aging, apoptosis, prostaglandin biosynthesis, estrogen activity, development of elasticity, development of tone, humectification, formation of age spots, crosslinking of collagen, oxidation of LDL and telomerase activity.

69. The method of claim 65, wherein said effect is selected from the group consisting of retarding aging, retarding apoptosis, inhibition of prostaglandin biosynthesis, agonism of estrogen activity, increasing elasticity, increasing tone, increasing humectification, dissipating age spots, retarding the process of collagen crosslinking, retarding LDL oxidation and stimulating telomerase activity.

70. The method of claim 65, wherein said step of administering is conducted in a manner selected from the group consisting of locally and systemically.

71. The method of claim 70, wherein said step of administering locally includes administration via an administration route selected from the group consisting of topical application to skin, intravaginal application, intrarectal application, intranasal application, intraocular application and inhalation.

72. The method of claim 70, wherein said step of administering systemically includes administration via an administration route selected from the group consisting of oral administration, injection, subcutaneous implantation, intravaginal application, intrarectal application, and inhalation.

73. The method of claim 65, wherein the pomegranate seed oil is administered together with at least one additional item selected from the group consisting of a pomegranate juice product and a pomegranate peel product.

74. The method of claim 65, wherein said effect includes an antioxidant effect.

75. The method of claim 74, wherein said antioxidant effect is at least partially produced by at least one item selected from the group consisting of 17 alpha estradiol, gamma tocopherol and campesterol.

76. An improved method of retarding the process of atherosclerosis, the method comprising the steps of::
(a) fermenting juice extracted from pomegranates;
(b) administering the fermented juice to a patient.

77. The method of claim 76, wherein said juice further contains a pomegranate peel product.

78. The method of claim 7763, wherein said pomegranate peel product includes residual pomegranate peel present in said juice when extracted from said pomegranates.

79. The method of claim 76, comprising the additional step of adding pomegranate peel to said juice.

80. The method of claim 76, wherein said step of fermenting serves to at least partially de-glycosylate flavonoids in said juice, thereby enhancing their bio-availability.

81. The method of claim 76, comprising the additional step of:
(c) further administering a pomegranate seed product with said fermented juice.

82. The method of claim 81, wherein said pomegranate seed product includes at least one item selected from pomegranate seed oil and an unsaponified fraction thereof.

83. A composition for inhibiting cellular aging, the composition comprising an effective amount of a pomegranate product.

84. The composition of claim 83, wherein said pomegranate product includes at least one item selected from the group consisting of a pomegranate seed product, a pomegranate juice product and a pomegranate peel product.

85. The composition of claim 84, wherein said pomegranate seed product includes at least one item selected from the group consisting of pomegranate seed oil, an unsaponified fraction thereof and an alcohol extract of pomegranate seed cake.

86. The composition of claim 84, wherein said pomegranate juice product includes at least one item selected from the group consisting of pomegranate juice, fermented pomegranate juice, dried pomegranate juice, dried fermented pomegranate juice, partially fermented pomegranate juice, partially dried pomegranate juice, partially fermented partially dried pomegranate juice, reduced pomegranate juice, partially reduced pomegranate juice and lyophylysates thereof.

87. The composition of claim 84, wherein said pomegranate peel product is selected from the group consisting of pomegranate peel residue present in pomegranate juice as a result of a juicing process, an aqueous extract of pomegranate peel, an alcohol extract of pomegranate peel, an extract performed with an organic solvent which is not alcohol, and a supercritical CO₂ extract of pomegranate peel.

88. The composition of claim 84, wherein said pomegranate peel product contains at least one item selected from the group consisting of quercetin, kaempferol, luteolin, derivatives thereof and combinations thereof.

89. The composition of claim 87, wherein said supercritical fluid extract is conducted with at least one item selected from the group consisting of CO₂, ethanol, methanol and water.
